SEQUENCE LISTING (117 sequences)

SEQ ID NO: 1 - protein, 456 aa, Klebsiella variicola
  misc_feature: cystathionine-beta-synthase
MSLFHSVSDLIGHTPLLQLHKLDTGPCSLFLKLENQNPGGSIKDRVALSMINEAERQGKLAPGGTIIEATAGNTGLGLALIAAQKNYRLILVVPDKMSREKIFHLRALGATVLLTRSDVNKGHPAYYQDYARRLADETPGAFYIDQFNNDANPLAHATSTAPELYQQLEGDIDAVVVGVGSGGTLGGLQAWFAEHSPKTEFILADPAGSILADQVDTGRYGETGSWLVEGIGEDFIPPLARLEGVHTAYRVSDSEAFQTARQLLQVEGVLAGSSTGTLLSAALRYCRAQSRPKRVVTFACDSGNKYLSKMFNDDWMRQQGLIARPEQGDLSDFIALRHDEGATVTAAPDDTLAAVFTRMRLYDISQLPVLEDGHVVGIVDEWDLIRHVQGDRQRFSLPVSEAMSRHVETLDKRAPESELQAILDRGLVAVIADNARFLGLVTRSDVLTAWRNRVAQ

SEQ ID NO: 2 - DNA, 1371 bp, Klebsiella variicola
  misc_feature: Coding sequence of SEQ ID NO 1
atgtcactgtttcattccgtgagcgacctgatcggtcatactcccctgctccagttgcacaaactggataccggcccctgcagtctgtttttaaaactggaaaatcagaatcccggcgggtcaattaaagatcgcgttgcgctgtcgatgattaatgaagccgagcgccagggaaaactggcgcccggaggaacgataatcgaagccaccgccggcaacaccggcctcggcctggcgctgatcgcggcgcagaaaaattaccggctgatcctggtggtgccggacaaaatgagccgcgagaaaatctttcacctgcgggcgctgggcgcgacggtgctgctcacccgttccgacgtcaataaaggccacccggcctattatcaggattatgcccgccgccttgccgatgaaacgccgggcgccttttatatcgaccagtttaataacgacgccaatccgctggcccatgccacctccaccgcgccggagctttatcagcagctggagggcgacattgacgccgtcgttgtcggcgtcggctccggcggcacgctgggcgggcttcaggcctggtttgctgaacactcgccgaaaacggaatttattctcgccgatccggcgggttcgatccttgccgatcaggttgataccggtcgctacggcgaaacgggatcctggctggtcgaagggatcggcgaagattttatcccgcctctggccaggctggagggggtgcataccgcgtatcgcgtcagcgacagcgaagcgttccagactgcgcgccagctgctgcaggtcgagggcgtgctggccggctcttccaccggcaccttgctcagcgccgccctgcgctactgccgggcacaaagccgtccgaaacgggtggtgaccttcgcctgtgatagcggcaataaatacctgtccaaaatgttcaacgatgactggatgcgccagcaaggactgatcgcccggcccgaacagggcgatctcagtgattttatcgccctgcgccatgacgaaggcgccaccgtcacggccgcgccggacgacaccctggccgcagtctttacccgcatgcggctgtatgacatctcccagctgccggtgctggaggatggccacgtggtcggcattgtcgatgagtgggatttgatccgccacgtacagggcgatcgccaacgcttctccctgccggtcagcgaagcgatgagccgccatgttgaaactctcgataagcgtgcgccagagagcgaactgcaggctattctcgatcgcggtctggtggcggtgatcgctgacaacgcgcgttttcttggcctggtcacccgcagcgatgttctaaccgcctggcgtaaccgagtcgcacaataa

SEQ ID NO: 3 - protein, 382 aa, Klebsiella variicola
  misc_feature: cystathionine-gamma-lyase
MSSIHTLSVHSGTFTDSHGAVMPPIYATSTFAQPAPGQHTGYEYSRSGNPTRHALETAIADLENGTRGYAFASGLAAISTVLELLDKDSHLVAVDDVYGGTYRLLENVRRRSAGLQVSWVKPDDLKGIEAAIRPDTRMIWVETPTNPLLKLADLSAIAAIARRHNLISVADNTFASPAIHRPLEHGFDIVVHSATKYLNGHSDVVAGLAVVGDNSELAEKLGYLQNAVGGVLDPFSSFLTLRGIRTLALRMERHSANALQLAEWLEQQPEVERVWFPWLASHPHHQLARQQMALPGGMISVVVKGDEGYAERIISKLRWFTLAESLGGVESLVSQPFSMTHASIPLEKRLANGITPQLIRLSVGIEDPNDLIADWQQALREE

SEQ ID NO: 4 - DNA, 1149 bp, Klebsiella variicola
  misc_feature: Coding sequence of SEQ ID NO 3
atgtcatctatccacaccctgagcgtacatagcggaaccttcaccgacagccacggcgcagtgatgccgccgatttacgccacctccacttttgctcagccggcgccggggcagcataccggctatgaatattcccgcagcgggaaccctacccgccacgcgctggagacggcgatcgccgacctggaaaacggcacgcgcggctacgcgttcgcctccgggctggccgccatctccaccgtgctggagctgctggataaggacagtcacctggtggcggtcgatgatgtgtatggcggcacttaccgcctgctggaaaacgtgcgccgccgtagcgctggtctgcaggtgagctgggtgaaacccgacgacctgaaggggattgaagcggcgatccgccccgatacgcgcatgatctgggtggaaacgcccaccaacccgctgctgaagctggcggatctcagcgccatcgccgccatcgcgcgacgccataacctgatcagcgtcgccgataacaccttcgcctcgccggcgatccatcgcccgctggagcatggatttgatattgtggtgcactccgcgaccaaatacctcaacggccattccgatgtcgtcgccggcctggcggtggttggcgataactccgagctcgcggaaaagcttggctatctgcagaatgcggtaggcggggtacttgacccgttcagcagcttcctgacgctacgtggtatccgtaccctggcgctgcggatggagcgccacagcgcgaacgcgctgcagctcgccgaatggctggaacagcagccggaagtggagcgagtctggttcccctggctggcctcgcacccgcatcatcagttggcgcggcagcaaatggcgctgcccggcggcatgatttcggtggtggtgaagggcgatgaaggttacgccgagcgcattatcagcaagctgcgctggtttacgcttgcggagagcctgggcggcgtggagagtctggtgagccagcccttcagcatgacccatgcctcgatcccgctggagaaacgtctggcgaatggcatcacgccccagctgatccgcctgtcggtggggatcgaagatcccaacgatctgattgccgactggcagcaggcgctgcgggaggagtga

SEQ ID NO: 5 - protein, 386 aa, Klebsiella variicola
  misc_feature: cystathionine-gamma-synthase
MTRKQATIAVRSGLNDDEQYGCVVPPIHLSSTYNFTGFNEPRAHDYSRRGNPTRDVVQRALAELEGGAGAVLTNTGMSAILLVTTVFLKPGDLLVAPHDCYGGSYRLFDSLAKRGCYRVQFVDQNDEQALRAALAEKPKLVLVESPSNPLLRVVDIAKICGLAREAGAVSVVDNTFLSPALQNPLALGADLVLHSCTKYLNGHSDVVAGVVIANDPATVTELAWWANNIGVTGGAFDSYLLLRGLRTLSPRMEVAQRNALAIVEYLKTQPLVKKLYHPSLPENQGHEIAARQQKGFGAMLSFELDGDEQTLRRFLSGLSLFTLAESLGGVESLISHAATMTHAGMAPEARAAAGISETLLRISTGIEDGEDLIADLENGFRAANEE

SEQ ID NO: 6 - DNA, 1126 bp, Klebsiella variicola
  misc_feature: Coding sequence of SEQ ID NO 5
atgacgcgtaaacaggccaccatcgcagtgcgtagcggtttaaatgacgacgagcagtacggttgcgttgtcccgccgatccacctttccagtacctataacttcaccggctttaatgaacctcgcgcccacgactactcccgccgcggcaacccgacgcgtgatgtggtgcagcgcgccttagcggagctggaaggtggcgccggcgcggtgctgaccaataccggtatgtcggcgatcctgctggtgacgacggtgttccttaagccgggcgatctgctggtggcgccgcacgactgctacggcggcagctaccgtctgttcgacagcctggcgaagcgcggctgttatcgcgtccagttcgtcgatcaaaatgatgagcaggcgctgcgggcggcgttggcggaaaaaccgaagctggtgctggtcgaaagcccaagtaatccattgctgcgggtggtggatattgcgaaaatctgtggtctggcgcgtgaggcaggcgcggtgagcgtggtggacaacaccttcctgagcccggcgctgcaaaacccgctggcgctgggtgccgatctggtgttgcattcctgtactaaatacctgaatggccactccgatgtggtggccggggtggtgattgccaacgatccggcgactgtcaccgaactggcatggtgggcgaataatattggcgtcaccggcggcgccttcgacagctatttgttgctgcgcggtctgcggacgctgtctccgcgtatggaagtggcgcagcgtaatgccctggcgattgttgagtatctgaaaacccagccgctggtgaaaaagctgtatcatccgtcgctgccggaaaaccaggggcatgaaattgcggcgcgtcagcaaaaaggttttggcgcaatgttaagcttcgagctggacggtgacgagcagacgctgcgccgtttcctgagcgggctgtcgctgtttacactggcggaatcgctggggggcgttgaaagcctgatttcccatgcggcaacgatgacgcatgccggcatggcgcctgaagcgcgtgccgccgccggaatttcagaaacgcttctgcgtatttcaaccggtattgaagatggtgaagatttaattgccgacc

SEQ ID NO: 7 - protein, 395 aa, Klebsiella variicola
  misc_feature: cystathionine-beta-lyase
MADKHLDTALVNAGRSKKYTQGSVNSVIQRASSLVFDTVEAKKHATRNRANGELFYGRRGTLTHFSLQEAMCELEGGAGCALFPCGAAAVANTILAFVEQGDHVLMTNTAYEPSQDFCTKILAKLGVATSWFDPLIGADIAQLIRPETRVVFLESPGSITMEVHDVPAIVAAVRQVAPEAIIMIDNTWAAGILFKALDFGIDISIQAGTKYLIGHSDAMVGTAVANARCWPQLRENAYLMGQMLDADTAYMTSRGLRTLGVRLRQHHESSLRIAEWLAQHPQVARVNHPALPGSKGHEFWKRDFTGSSGLFSFVLNKRLNDAELAAYLDNFSLFSMAYSWGGFESLILANQPEQIAHIRPDAEVDFSGTLIRLHIGLENVDDLQADLAAGFARIV

SEQ ID NO: 8 - DNA, 1188 bp, Klebsiella variicola
  misc_feature: Coding sequence of SEQ ID NO 7
atggctgataaacatcttgataccgcgctggtcaacgccggacggagcaaaaaatatacccagggctcagtgaacagcgtgatccagcgcgcgtcatcgctggttttcgacaccgtagaggccaaaaagcacgccacccgcaatcgggccaacggcgagctgttctacgggcgtcgcggcaccctgacccacttctcgctgcaggaagcgatgtgcgagctggaaggcggcgccggctgcgccctcttcccctgtggcgcggccgcggtggccaacaccatcctggcgttcgtcgaacagggcgaccatgtgctgatgaccaataccgcctatgagccaagccaggatttttgtaccaaaattctcgctaaactcggcgtcgccaccagctggttcgatcccctcatcggcgccgatattgcccaacttattcggccggagacccgcgtggtgttccttgaatcgcctggctccatcaccatggaagtgcatgatgtgccagcgattgtcgccgccgtgcgtcaggtcgcgccggaagcgattatcatgatcgataacacctgggcggcggggatcctgttcaaagcgctggattttggcatcgatatttctattcaggcaggtaccaaatacctgatcggccattccgacgccatggtcggcactgcggtggcgaacgcgcgctgctggccgcagctgcgtgagaatgcctatctgatgggacaaatgctggacgccgataccgcctatatgaccagccgcggcctgcgcaccctgggcgtgcgcctgcgtcagcatcatgaaagcagcctgcgcatcgccgagtggctggcgcagcatccgcaggtggcgcgggtcaaccaccccgccctgcccggcagtaaaggccatgagttctggaagcgcgattttaccggcagcagcgggctgttctcgtttgtgctcaacaagcgtctcaacgacgctgagctggcagcgtatctcgacaacttcagcctgttcagcatggcctactcgtggggcggctttgaatcgctgattctggccaatcagccggagcaaatcgctcacatccgaccggatgcggaagtcgacttcagcggcacgttgatccgtttgcacattggtctcgaaaatgttgacgatctgcaggcggatttagcagcgggcttcgcgcgtatcgtgtaa

SEQ ID NO: 9 - protein, 232 aa, Klebsiella variicola
  misc_feature: adenosylhomocysteine nucleosidase
MKIGIIGAMEEEVTLLRDKIENRQTISLGGCEIYTGQLNGTEVALLKSGIGKVAAALGATLLLEHCKPDVIINTGSAGGLAPTLKVGDIVVSDEARYHDADVTAFGYEYGQLPGCPAGFKADDKLIAAAEACIAELNLNAVRGLIVSGDAFINGSVGLAKIRHNFPQAIAVEMEATAIAHVCHNFNVPFVVVRAISDVADQQSHLSFDEFLAVAAKQSSLMVESLVQKLAHG

SEQ ID NO: 10 - DNA, 699 bp, Klebsiella variicola
  misc_feature: Coding sequence of SEQ ID NO 9
atgaaaatcggcatcattggtgcaatggaagaagaagttacgctgctgcgtgacaaaatcgaaaaccgtcaaactatcagtctcggcggttgcgaaatctataccggccaactgaatggaaccgaggttgcgcttctgaaatcgggcatcggtaaagtcgctgcggcgctgggtgccactttgctgttggaacactgcaagccagatgtgattattaacaccggttctgccggtggcctggcaccaacgttgaaagtgggcgatatcgttgtctcggacgaagcacgttatcacgacgcggatgtcacggcatttggttatgaatacggtcagttaccaggctgtccggcaggctttaaagctgacgataaactgatcgctgccgctgaggcctgcattgccgaactgaatcttaacgctgtacgtggcctgattgttagcggcgacgctttcatcaacggttctgttggtctggcgaaaatccgccacaacttcccacaggccattgctgtagagatggaagcgacggcaatcgcccatgtctgccacaatttcaacgtcccgtttgttgtcgtacgcgccatctccgacgtggccgatcaacagtctcatcttagcttcgatgagttcctggctgttgccgctaaacagtccagcctgatggttgagtcactggtgcagaaacttgcacatggctaa

SEQ ID NO: 11 - DNA, 2531 bp, Klebsiella variicola
  misc_feature (1)..(1371): SEQ ID NO2
  misc_feature (1383)..(2531): SEQ ID NO4
atgtcactgtttcattccgtgagcgacctgatcggtcatactcccctgctccagttgcacaaactggataccggcccctgcagtctgtttttaaaactggaaaatcagaatcccggcgggtcaattaaagatcgcgttgcgctgtcgatgattaatgaagccgagcgccagggaaaactggcgcccggaggaacgataatcgaagccaccgccggcaacaccggcctcggcctggcgctgatcgcggcgcagaaaaattaccggctgatcctggtggtgccggacaaaatgagccgcgagaaaatctttcacctgcgggcgctgggcgcgacggtgctgctcacccgttccgacgtcaataaaggccacccggcctattatcaggattatgcccgccgccttgccgatgaaacgccgggcgccttttatatcgaccagtttaataacgacgccaatccgctggcccatgccacctccaccgcgccggagctttatcagcagctggagggcgacattgacgccgtcgttgtcggcgtcggctccggcggcacgctgggcgggcttcaggcctggtttgctgaacactcgccgaaaacggaatttattctcgccgatccggcgggttcgatccttgccgatcaggttgataccggtcgctacggcgaaacgggatcctggctggtcgaagggatcggcgaagattttatcccgcctctggccaggctggagggggtgcataccgcgtatcgcgtcagcgacagcgaagcgttccagactgcgcgccagctgctgcaggtcgagggcgtgctggccggctcttccaccggcaccttgctcagcgccgccctgcgctactgccgggcacaaagccgtccgaaacgggtggtgaccttcgcctgtgatagcggcaataaatacctgtccaaaatgttcaacgatgactggatgcgccagcaaggactgatcgcccggcccgaacagggcgatctcagtgattttatcgccctgcgccatgacgaaggcgccaccgtcacggccgcgccggacgacaccctggccgcagtctttacccgcatgcggctgtatgacatctcccagctgccggtgctggaggatggccacgtggtcggcattgtcgatgagtgggatttgatccgccacgtacagggcgatcgccaacgcttctccctgccggtcagcgaagcgatgagccgccatgttgaaactctcgataagcgtgcgccagagagcgaactgcaggctattctcgatcgcggtctggtggcggtgatcgctgacaacgcgcgttttcttggcctggtcacccgcagcgatgttctaaccgcctggcgtaaccgagtcgcacaataaggaatttttttatgtcatctatccacaccctgagcgtacatagcggaaccttcaccgacagccacggcgcagtgatgccgccgatttacgccacctccacttttgctcagccggcgccggggcagcataccggctatgaatattcccgcagcgggaaccctacccgccacgcgctggagacggcgatcgccgacctggaaaacggcacgcgcggctacgcgttcgcctccgggctggccgccatctccaccgtgctggagctgctggataaggacagtcacctggtggcggtcgatgatgtgtatggcggcacttaccgcctgctggaaaacgtgcgccgccgtagcgctggtctgcaggtgagctgggtgaaacccgacgacctgaaggggattgaagcggcgatccgccccgatacgcgcatgatctgggtggaaacgcccaccaacccgctgctgaagctggcggatctcagcgccatcgccgccatcgcgcgacgccataacctgatcagcgtcgccgataacaccttcgcctcgccggcgatccatcgcccgctggagcatggatttgatattgtggtgcactccgcgaccaaatacctcaacggccattccgatgtcgtcgccggcctggcggtggttggcgataactccgagctcgcggaaaagcttggctatctgcagaatgcggtaggcggggtacttgacccgttcagcagcttcctgacgctacgtggtatccgtaccctggcgctgcggatggagcgccacagcgcgaacgcgctgcagctcgccgaatggctggaacagcagccggaagtggagcgagtctggttcccctggctggcctcgcacccgcatcatcagttggcgcggcagcaaatggcgctgcccggcggcatgatttcggtggtggtgaagggcgatgaaggttacgccgagcgcattatcagcaagctgcgctggtttacgcttgcggagagcctgggcggcgtggagagtctggtgagccagcccttcagcatgacccatgcctcgatcccgctggagaaacgtctggcgaatggcatcacgccccagctgatccgcctgtcggtggggatcgaagatcccaacgatctgattgccgactggcagcaggcgctgcgggaggagtga

SEQ ID NO: 12 - DNA, 28 bp, Artificial
  Forward Cloning Primer for CBS-CGL
  misc_feature (1)..(4): n is a, c, g, or t
nnnntctagaatgtcactgtttcattcc

SEQ ID NO: 13 - DNA, 28 bp, Artificial
  Reverse cloning primer for CBS-CGL
  misc_feature (1)..(4): n is a, c, g, or t
nnnngaattccggaataatcactcctcc

SEQ ID NO: 14 - DNA, 19 bp, Artificial
  Sequencing primer for CBS-CGL
accacttattcagcgaacc

SEQ ID NO: 15 - DNA, 19 bp, Artificial
  Sequencing primer for CBS-CGL
ggaggaacgataatcgaag

SEQ ID NO: 16 - DNA, 18 bp, artificial
  Sequencing primer for CBS-CGL
gatcctggctggtcgaag

SEQ ID NO: 17 - DNA, 18 bp, artificial
  Sequencing primer for CCBS-CGL
ccaacgcttctccctgcc

SEQ ID NO: 18 - DNA, 18 bp, artificial
  Sequencing primer for CBS-CGL
tggataaggacagtcacc

SEQ ID NO: 19 - DNA, 18 bp, artificial
  Sequencing primer for CBS-CGL
gaagtggagcgagtctgg

SEQ ID NO: 20 - protein, 551 aa, Homo sapiens
  misc_feature: cystathionine-beta-synthase like
MPSETPQAEVGPTGCPHRSGPHSAKGSLEKGSPEDKEAKEPLWIRPDAPSRCTWQLGRPASESPHHHTAPAKSPKILPDILKKIGDTPMVRINKIGKKFGLKCELLAKCEFFNAGGSVKDRISLRMIEDAERDGTLKPGDTIIEPTSGNTGIGLALAAAVRGYRCIIVMPEKMSSEKVDVLRALGAEIVRTPTNARFDSPESHVGVAWRLKNEIPNSHILDQYRNASNPLAHYDTTADEILQQCDGKLDMLVASVGTGGTITGIARKLKEKCPGCRIIGVDPEGSILAEPEELNQTEQTTYEVEGIGYDFIPTVLDRTVVDKWFKSNDEEAFTFARMLIAQEGLLCGGSAGSTVAVAVKAAQELQEGQRCVVILPDSVRNYMTKFLSDRWMLQKGFLKEEDLTEKKPWWWHLRVQELGLSAPLTVLPTITCGHTIEILREKGFDQAPVVDEAGVILGMVTLGNMLSSLLAGKVQPSDQVGKVIYKQFKQIRLTDTLGRLSHILEMDHFALVVHEQIQYHSTGKSSQRQMVFGVVTAIDLLNFVAAQERDQK

SEQ ID NO: 21 - DNA, 1656 bp, Homo sapiens
  misc_feature: Coding sequence of SEQ ID NO 20
  misc_feature: cDNA
atgccttctgagaccccccaggcagaagtggggcccacaggctgcccccaccgctcagggccacactcggcgaaggggagcctggagaaggggtccccagaggataaggaagccaaggagcccctgtggatccggcccgatgctccgagcaggtgcacctggcagctgggccggcctgcctccgagtccccacatcaccacactgccccggcaaaatctccaaaaatcttgccagatattctgaagaaaatcggggacacccctatggtcagaatcaacaagattgggaagaagttcggcctgaagtgtgagctcttggccaagtgtgagttcttcaacgcgggcgggagcgtgaaggaccgcatcagcctgcggatgattgaggatgctgagcgcgacgggacgctgaagcccggggacacgattatcgagccgacatccgggaacaccgggatcgggctggccctggctgcggcagtgaggggctatcgctgcatcatcgtgatgccagagaagatgagctccgagaaggtggacgtgctgcgggcactgggggctgagattgtgaggacgcccaccaatgccaggttcgactccccggagtcacacgtgggggtggcctggcggctgaagaacgaaatccccaattctcacatcctagaccagtaccgcaacgccagcaaccccctggctcactatgacaccaccgctgatgagatcctgcagcagtgtgatgggaagctggacatgctggtggcttcagtgggcacgggcggcaccatcacgggcattgccaggaagctgaaggagaagtgtcctggatgcaggatcattggggtggatcccgaagggtccatcctcgcagagccggaggagctgaaccagacggagcagacaacctacgaggtggaagggatcggctacgacttcatccccacggtgctggacaggacggtggtggacaagtggttcaagagcaacgatgaggaggcgttcacctttgcccgcatgctgatcgcgcaagaggggctgctgtgcggtggcagtgctggcagcacggtggcggtggccgtgaaggccgcgcaggagctgcaggagggccagcgctgcgtggtcattctgcccgactcagtgcggaactacatgaccaagttcctgagcgacaggtggatgctgcagaagggctttctgaaggaggaggacctcacggagaagaagccctggtggtggcacctccgtgttcaggagctgggcctgtcagccccgctgaccgtgctcccgaccatcacctgtgggcacaccatcgagatcctccgggagaagggcttcgaccaggcgcccgtggtggatgaggcgggggtaatcctgggaatggtgacgcttgggaacatgctctcgtccctgcttgccgggaaggtgcagccgtcagaccaagttggcaaagtcatctacaagcagttcaaacagatccgcctcacggacacgctgggcaggctctcgcacatcctggagatggaccacttcgccctggtggtgcacgagcagatccagtaccacagcaccgggaagtccagtcagcggcagatggtgttcggggtggtcaccgccattgacttgctgaacttcgtggccgcccaggagcgggaccagaagtga

SEQ ID NO: 22 - protein, 551 aa, Homo sapiens; identical to SEQ ID NO: 20
  misc_feature: cystathionine-beta-synthase

SEQ ID NO: 23 - DNA, 1656 bp, Homo Sapiens
  misc_feature: Coding sequence of SEQ ID NO 22
  misc_feature: cDNA
atgccttctgagaccccccaggcagaagtggggcccacaggctgcccccaccgctcagggccacactcggcgaaggggagcctggagaaggggtccccagaggataaggaagccaaggagcccctgtggatccggcccgatgctccgagcaggtgcacctggcagctgggccggcctgcctccgagtccccacatcaccacactgccccggcaaaatctccaaaaatcttgccagatattctgaagaaaatcggggacacccctatggtcagaatcaacaagattgggaagaagttcggcctgaagtgtgagctcttggccaagtgtgagttcttcaacgcgggcgggagcgtgaaggaccgcatcagcctgcggatgattgaggatgctgagcgcgacgggacgctgaagcccggggacacgattatcgagccgacatccgggaacaccgggatcgggctggccctggctgcggcagtgaggggctatcgctgcatcatcgtgatgccagagaagatgagctccgagaaggtggacgtgctgcgggcactgggggctgagattgtgaggacgcccaccaatgccaggttcgactccccggagtcacacgtgggggtggcctggcggctgaagaacgaaatccccaattctcacatcctagaccagtaccgcaacgccagcaaccccctggctcactacgacaccaccgctgatgagatcctgcagcagtgtgatgggaagctggacatgctggtggcttcagtgggcacgggcggcaccatcacgggcattgccaggaagctgaaggagaagtgtcctggatgcaggatcattggggtggatcccgaagggtccatcctcgcagagccggaggagctgaaccagacggagcagacaacctacgaggtggaagggatcggctacgacttcatccccacggtgctggacaggacggtggtggacaagtggttcaagagcaacgatgaggaggcgttcacctttgcccgcatgctgatcgcgcaagaggggctgctgtgcggtggcagtgctggcagcacggtggcggtggccgtgaaggccgcgcaggagctgcaggagggccagcgctgcgtggtcattctgcccgactcagtgcggaactacatgaccaagttcctgagcgacaggtggatgctgcagaagggctttctgaaggaggaggacctcacggagaagaagccctggtggtggcacctccgtgttcaggagctgggcctgtcagccccgctgaccgtgctcccgaccatcacctgtgggcacaccatcgagatcctccgggagaagggcttcgaccaggcgcccgtggtggatgaggcgggggtaatcctgggaatggtgacgcttgggaacatgctctcgtccctgcttgccgggaaggtgcagccgtcagaccaagttggcaaagtcatctacaagcagttcaaacagatccgcctcacggacacgctgggcaggctctcgcacatcctggagatggaccacttcgccctggtggtgcacgagcagatccagtaccacagcaccgggaagtccagtcagcggcagatggtgttcggggtggtcaccgccattgacttgctgaacttcgtggccgcccaggagcgggaccagaagtga

SEQ ID NO: 24 - protein, 405 aa, Homo sapiens
  misc_feature: cystathionine-gamma-lyase
MQEKDASSQGFLPHFQHFATQAIHVGQDPEQWTSRAVVPPISLSTTFKQGAPGQHSGFEYSRSGNPTRNCLEKAVAALDGAKYCLAFASGLAATVTITHLLKAGDQIICMDDVYGGTNRYFRQVASEFGLKISFVDCSKIKLLEAAITPETKLVWIETPTNPTQKVIDIEGCAHIVHKHGDIILVVDNTFMSPYFQRPLALGADISMYSATKYMNGHSDVVMGLVSVNCESLHNRLRFLQNSLGAVPSPIDCYLCNRGLKTLHVRMEKHFKNGMAVAQFLESNPWVEKVIYPGLPSHPQHELVKRQCTGCTGMVTFYIKGTLQHAEIFLKNLKLFTLAESLGGFESLAELPAIMTHASVLKNDRDVLGISDTLIRLSVGLEDEEDLLEDLDQALKAAHPPSGSHS

SEQ ID NO: 25 - DNA, 1218 bp, Homo sapiens
  misc_feature: Coding sequence of SEQ ID NO 24
  misc_feature: cDNA
atgcaggaaaaagacgcctcctcacaaggtttcctgccacacttccaacatttcgccacgcaggcgatccatgtgggccaggatccagagcaatggacctccagggctgtagtgccccccatctcactgtccaccacgttcaagcaaggggcgcctggccagcactcgggttttgaatatagccgttctggaaatcccactaggaattgccttgaaaaagcagtggcagcactggatggggctaagtactgtttggcctttgcttcaggtttagcagccactgtaactattacccatcttttaaaagcaggagaccaaattatttgtatggatgatgtgtatggaggtacaaacaggtacttcaggcaagtggcatctgaatttggattaaagatttcttttgttgattgttccaaaatcaaattactagaggcagcaattacaccagaaaccaagcttgtttggatcgaaacccccacaaaccccacccagaaggtgattgacattgaaggctgtgcacatattgtccataagcatggagacattattttggtcgtggataacacttttatgtcaccatatttccagcgccctttggctctgggagctgatatttctatgtattctgcaacaaaatacatgaatggccacagtgatgttgtaatgggcctggtgtctgttaattgtgaaagccttcataatagacttcgtttcttgcaaaactctcttggagcagttccatctcctattgattgttacctctgcaatcgaggtctgaagactctacatgtccgaatggaaaagcatttcaaaaacggaatggcagttgcccagttcctggaatctaatccttgggtagaaaaggttatttatcctgggctgccctctcatccacagcatgagttggtgaagcgtcagtgtacaggttgtacagggatggtcaccttttatattaagggcactcttcagcatgctgagattttcctcaagaacctaaagctatttactctggccgagagcttgggaggattcgaaagccttgctgagcttccggcaatcatgactcatgcatcagttcttaagaatgacagagatgtccttggaattagtgacacactgattcgactttctgtgggcttagaggatgaggaagacctactggaagatctagatcaagctttgaaggcagcacaccctccaagtggaagtcacagctag

SEQ ID NO: 26 - protein, 561 aa, Mus musculus
  misc_feature: cystathionine-beta-synthase
MPSGTSQCEDGSAGGFQHLDMHSEKRQLEKGPSGDKDRVWIRPDTPSRCTWQLGRAMADSPHYHTVLTKSPKILPDILRKIGNTPMVRINKISKNAGLKCELLAKCEFFNAGGSVKDRISLRMIEDAERAGNLKPGDTIIEPTSGNTGIGLALAAAVKGYRCIIVMPEKMSMEKVDVLRALGAEIVRTPTNARFDSPESHVGVAWRLKNEIPNSHILDQYRNASNPLAHYDDTAEEILQQCDGKLDMLVASAGTGGTITGIARKLKEKCPGCKIIGVDPEGSILAEPEELNQTEQTAYEVEGIGYDFIPTVLDRAVVDKWFKSNDEDSFAFARMLIAQEGLLCGGSSGSAMAVAVKAARELQEGQRCVVILPDSVRNYMSKFLSDKWMLQKGFMKEELSVKRPWWWRLRVQELSLSAPLTVLPTVTCEDTIAILREKGFDQAPVVNESGAILGMVTLGNMLSSLLAGKVRPSDEVCKVLYKQFKPIHLTDTLGTLSHILEMDHFALVVHEQIQSRDQAWSGVVGGPTDCSNGMSSKQQMVFGVVTAIDLLNFVAAREQTQT

SEQ ID NO: 27 - DNA, 1686 bp, Mus musculus
  misc_feature: Coding sequence of SEQ ID NO 26
  misc_feature: cDNA
atgccttcagggacatcccagtgtgaagatggctctgctgggggcttccagcacttggacatgcactcagaaaagagacaactggagaagggcccctcaggggacaaggatcgagtctggatccggcctgataccccaagcagatgtacctggcagctgggcagggccatggcggactccccacattatcacacagtgctgaccaaatcccccaaaattttaccagatattctgaggaaaattgggaacacccctatggtcagaatcaacaagatctcaaagaatgccggtctcaagtgtgagctcttggccaagtgtgagttcttcaatgcgggtgggagtgtgaaggaccgcatcagccttcggatgatcgaagatgctgagcgagctggaaacttgaagcctggagacactatcattgagccaacttctggcaacacagggatcgggctggctctggctgctgcagtgaagggctatcgctgcattatcgtgatgccggagaagatgagtatggagaaggtggatgtgctgcgggctctgggagccgagattgtgaggacgcccaccaatgccagatttgattcccccgagtcccacgtgggagtggcatggcgactgaagaacgaaatccctaattctcacattctggaccagtaccgcaatgccagcaaccctttggcacactacgatgacaccgccgaggagatcctgcagcagtgtgacgggaagctggatatgctggtggcttcagcaggcacgggtggcaccatcacagggatcgccagaaagctgaaggagaagtgccctggctgtaaaatcatcggtgtcgatcctgaaggctccatccttgcggagcccgaggagctgaaccagacggagcaaacagcctatgaggtggaagggattggctacgacttcatcccgacagtcctggacagggcggtggtggataagtggttcaagagcaacgatgaagattccttcgcctttgcccgcatgctcatcgcacaggaaggactgctatgtggtggaagctctggcagcgccatggctgtggctgtgaaggctgcccgggagctgcaggaagggcagcgctgtgtggtcatcctgcctgactctgtgcggaactacatgtccaagttcctgagtgacaaatggatgctgcagaaaggtttcatgaaagaggagctctcagtgaagaggccctggtggtggcgtctgcgtgttcaagagctgagcctgtcggccccgctgaccgtgttgcccacggtcacctgtgaggacaccatcgccatcctccgggagaagggttttgaccaggcacctgtggtcaacgagtctggggccatcctagggatggtgaccctcgggaacatgctgtcatccctgctggctggaaaggtgcggccatcagacgaagtctgcaaagtcctctacaagcagttcaaaccgatccacctgaccgacacgctgggcacactctctcacatcctggagatggaccacttcgccctggtggtccacgagcagatccaatcacgagaccaggcctggtcaggagtggtgggggggcccacagactgcagcaatggcatgtccagcaagcagcagatggtgtttggggttgtcactgccattgacctgctaaacttcgtggcagcccgtgagcagacccagacatag

SEQ ID NO: 28 - protein, 398 aa, Mus musculus
  misc_feature: cystathionine-gamma-lyase
MQKDASLSGFLPSFQHFATQAIHVGQEPEQWNSRAVVLPISLATTFKQDFPGQSSGFEYSRSGNPTRNCLEKAVAALDGAKHSLAFASGLAATITITHLLKAGDEIICMDEVYGGTNRYFRRVASEFGLKISFVDCSKTKLLEAAITPQTKLVWIETPTNPTLKLADIGACAQIVHKRGDIILVVDNTFMSAYFQRPLALGADICMCSATKYMNGHSDVVMGLVSVNSDDLNSRLRFLQNSLGAVPSPFDCYLCCRGLKTLQVRMEKHFKNGMAVARFLETNPRVEKVVYPGLPSHPQHELAKRQCSGCPGMVSFYIKGALQHAKAFLKNLKLFTLAESLGGYESLAELPAIMTHASVPEKDRATLGINDTLIRLSVGLEDEQDLLEDLDRALKAAHP

SEQ ID NO: 29 - DNA, 1197 bp, Mus musculus
  misc_feature: Coding sequence of SEQ ID NO 28
  misc_feature: cDNA
atgcagaaggacgcctctttgagcggcttcctgcctagtttccagcatttcgccactcaggccatccacgtgggacaagagcctgagcaatggaattctcgtgccgtggtgctgcccatttcgttggccaccacatttaagcaggacttcccgggccagtcctcgggttttgaatacagccgctctggaaatccaacaaggaattgcttggaaaaagcagtggctgcgttggatggggcaaagcacagtttggcctttgcatcgggtcttgctgccaccattacgattacccatcttttaaaagcaggagatgaaatcatttgcatggatgaagtgtatggaggcaccaacaggtacttcaggagggtggcatctgaatttggactgaagatttcttttgtagattgttccaaaaccaaattgctagaggcagcgattacaccacaaaccaagcttgtttggatcgaaacacccacaaacccaactttgaagttggctgacattggagcctgcgcacaaattgtccacaaacgtggagacatcattttggttgtagataacaccttcatgtctgcatatttccagagacctttggctctgggtgctgatatttgtatgtgttctgccacaaaatacatgaatggccacagcgatgttgtcatgggtttagtgtctgttaattctgatgacctcaatagtcggcttcgtttcctgcagaattcactaggagcagttccttctccttttgattgttacctctgctgccgaggcctgaagacactgcaggtccggatggagaaacatttcaagaatgggatggcggtggctcgtttcctggagaccaatccccgggtagaaaaggttgtttatcctgggctaccctctcaccctcagcatgagctggccaaacgccagtgctcgggctgcccagggatggtcagtttctacatcaagggtgctctgcagcatgctaaggccttcctcaaaaatctaaagctgtttactctggcagagagcctgggaggatatgagagtctggctgagcttccagcaatcatgacccatgcctctgtgcctgagaaggacagagctaccctcgggatcaatgacacactgatacgactttctgtgggcctagaggatgaacaggaccttcttgaagacctggatcgagctttgaaggcagcgcacccttaa

SEQ ID NO: 30 - protein, 457 aa, Pseudomonas aeruginosa
  misc_feature: cystathionine-beta-synthase
MTSDSRPAVLDLIGNTPLVRVTRFDTGPCTLYLKLESQNPGGSIKDRIGVAMIEAAERDGRLRPGGTIVEATAGNTGLGLALVGRAKGYRVVLVVPDKMSTEKVLHLRAMGAEVHITRSDVGKGHPEYYQDVAARLAQDIPGAFFADQFNNPANPLAHECGTGPELWAQTGHDLDAIVVGVGSSGTLTGLTRFFQKVQPELEMVLADPEGSIMAEYSRSGTLGTPGSWAVEGIGEDFVPAIADLSSVRHAYSISDEESFAMARELLRVEGIPGGSSTGTLLAAALRFCREQKEPKRVVSFVCDTGTRYLSKIYNDQWMTDQGLLQRKHYGDLRDIIARRFEEGRVISVGPDDTLLTAFQRMRLADVSQLPVLDDGKLVGVIDESDILLGVHADAPRFRDAVSSAMNAAPETLAPGASLAQLQAVLDRGLVAIVADASGFHGLITRFDLLNHLRRKLA

SEQ ID NO: 31 - DNA, 1374 bp, Pseudomonas aeruginosa
  misc_feature: Coding sequence of SEQ ID NO 30
atgacttccgactcacgtcccgccgtgctcgacctgatcggcaacaccccgctggttcgcgtgacgcgcttcgataccggcccctgcaccctgtacctcaagctcgaatcgcagaaccccggcggctccatcaaggatcgcatcggtgtcgccatgatcgaggccgccgagcgtgacggccgcctgcggcccggtggcaccatcgtcgaggccaccgccggcaacaccggtctcggcctggccctggtcggccgcgcgaagggctaccgggtggtgctggtggtgccggacaagatgtccaccgagaaggtcctgcacctgcgcgccatgggcgccgaggtgcacatcacccgatcggacgtcggcaagggccatccggagtactaccaggacgttgccgcgcgcctggcgcaggatattcccggcgccttcttcgccgaccagttcaacaacccggccaacccgctggcccacgagtgcggcaccggcccggagctctgggcgcagaccggccacgatctcgacgccatcgtcgtcggcgtcggctcctccggtaccctgaccggccttacccggttcttccagaaggtccagccggagctggagatggtgctcgccgatcccgagggctcgatcatggccgagtactcccgctccggcaccctgggtacgccgggttcctgggcggtggagggcatcggcgaggacttcgtaccggccatcgccgacctctccagcgtgcgccatgcctattccatcagcgacgaggaaagcttcgccatggcccgcgaactgttgcgcgtcgagggcattcccggcggctcctccaccggcaccctgctggccgcggcgctgcgcttctgtcgcgagcagaaggagccgaagcgggtggtcagcttcgtctgcgacaccggcacccgctacctgtcgaagatctacaacgaccagtggatgaccgaccagggcctgctgcaacgcaagcactacggcgacctgcgcgacatcatcgcgcggcgcttcgaggagggccgggtgatcagcgtcggtccggacgacaccctgctcaccgccttccagcgcatgcgcctggccgacgtttcgcaattgccggtgctggacgatggaaaactggtcggtgtgatcgacgagtcggatatcctgctcggtgtccacgccgacgcgccacgcttccgcgatgcggtaagcagcgcgatgaacgccgcgccggagaccctcgcgcccggcgccagcctggcccagttgcaggcggtcctggaccgcggcctggtggcgatcgttgccgatgccagcggcttccacggcctgatcacccgtttcgacctgttgaaccatttgcggagaaagctcgcatga

SEQ ID NO: 32 - protein, 394 aa, Pseudomonas aeruginosa
  misc_feature: cystathionine-gamma-lyase
MSQHDQHPDAPAQAFATRVIHAGQAPDPSTGAIMPPIYANSTYIQESPGVHKGLDYGRSHNPTRWALERCVADLEGGTQAFAFASGLAAISSVLELLDAGSHIVSGNDLYGGTFRLFERVRRRSAGHRFSFVDPTDLQAFEAALTPETRMVWVETPSNPLLRLTDLRAIAQLCRARGIISVADNTFASPYIQRPLELGFDVVVHSTTKYLNGHSDVIGGIAIVGDNPDLRERLGFLQNSVGAISGPFDAFLTLRGVKTLALRMERHCSNALALAQWLERQPQVARVYYPGLASHPQHELAKRQMRGFGGMISLDLRCDLAGARRFLENVRIFSLAESLGGVESLIEHPAIMTHASIPAETRADLGIGDSLIRLSVGVEALEDLQADLAQALAKI

SEQ ID NO: 33 - DNA, 1185 bp, Pseudomonas aeruginosa
  misc_feature: Coding sequence of SEQ ID NO 32
atgagccagcacgaccagcatcccgacgccccggcccaggcctttgcgacccgggtcatccacgccgggcaggcccccgatccctcgaccggcgcgatcatgccgccgatctacgccaactccacctatatccaggaaagccccggggtacacaaggggctcgactacggccgctcccacaacccgacccgctgggccctggagcgctgcgtggccgacctcgagggcggcacccaggccttcgccttcgcctccggcctggcggcgatctcctcggtgctggagctgctcgacgccggttcccacatcgtttccggcaatgacctctacggcggtaccttccgcctgttcgagcgggtccgccggcgcagcgccgggcatcgcttcagcttcgtcgacccgaccgacctgcaggccttcgaggccgcgctgaccccggaaacccgcatggtctgggtggaaaccccgagcaacccgctgctgcgcctcaccgacctgcgcgccatcgcccagctctgccgcgcccgcggcatcatcagcgtggccgacaacaccttcgccagcccctacatccagcgccccctggagctgggcttcgacgtggtggtgcactccaccaccaagtacctgaacggccattccgacgtgatcggcggcatcgccatcgtcggcgacaacccggacctgcgcgagcgcctgggcttcctgcagaactcggtgggcgccatctccgggccgttcgacgccttcctcaccctgcgcggggtgaagaccctggcgctgcgcatggagcgccattgcagtaacgccctggccctggcccagtggctggagcgccagccgcaagtggcgcgggtctactaccccggcctggcttcgcacccgcagcatgagctggcgaaacggcagatgcgtggcttcggcgggatgatctccctcgacctgcgttgcgacctggccggcgcccggcgcttcctggagaacgtgcggatcttctccctggcggagagcctgggcggggtggaaagcctgatcgagcatccggcgatcatgacccacgccagcatcccggcggaaacccgcgccgacctgggcattggtgactcgctgatccgcctgtcggtgggcgtcgaggcgctggaggacctgcaggccgacctggcgcaggcgctggcgaagatctga

SEQ ID NO: 34 - protein, 463 aa, Streptomyces venezuelae
  misc_feature: cystathionine-beta-synthase
MQFHDSMISLVGNTPLVKLNNVTAGIQATVLAKVEYFNPGGSVKDRIAVRMIEAAEQSGELKPGGTIVEPTSGNTGVGLAIVAQQKGYKCIFVCPDKVSLDKINVLRAYGAEVVVCPTAVDPEHPDSYYNVSDRLVRETPGAWKPDQYSNPNNPRSHYETTGPELWEQTDGKITHFVAGVGTGGTISGTGNYLKEASGGSVKIIGADPEGSVYSGGSGRPYLVEGVGEDFWPTAYDRNVTDRIVAVSDKDSFQMTRRLAKEEGLLVGGSCGMAVVAALEVARELGPDDVVVVLLPDSGRGYMSKIFSDEWMAGHGFLEDTSSATVADVLRHKEGGTMPSLVHMHPDETVGQAIEVLREYGVSQMPIVKPGAGHPDVMAAEVVGSVVERELLDALFTQRASLEDSLESHMSAPLPQVGSGEPVAALMSVLGEAADAAIVLVEGKPTGVVSRQDLLAFLANGGAK

SEQ ID NO: 35 - DNA, 1392 bp, Streptomyces venezuelae
  misc_feature: Coding sequence of SEQ ID NO 34
gtgcaattccacgactcgatgatcagcctcgtcggcaacaccccgctggtgaagctcaacaacgtcacagcgggcattcaggcgaccgtcctggccaaggtcgagtacttcaaccccggaggctcggtcaaggaccggatcgccgtgcggatgatcgaggcggccgagcagagcggcgagctcaagcccggtgggaccatcgtcgagcccacgtccggcaacaccggcgtcggcctggcgatcgtggcccagcagaagggctacaagtgcatcttcgtctgccccgacaaggtgtccctcgacaagatcaacgtgctgcgggcgtacggcgccgaggtcgtcgtctgccccaccgcggtcgacccggagcacccggactcgtactacaacgtctcggaccggctcgtccgtgagacgccgggcgcctggaagcccgaccagtactccaacccgaacaacccgcgctcccactacgagaccaccggtcccgagctctgggagcagacggacgggaagatcacccacttcgtggcgggcgtcggcaccggcggcaccatctccggcaccggcaactacctcaaggaggccagcggcggctccgtgaagatcatcggcgccgacccggagggctccgtctactccggcggctccgggcgcccgtacctcgtcgagggcgtcggcgaggacttctggccgaccgcctacgaccggaacgtcacggaccggatcgtcgccgtgtccgacaaggactccttccagatgacccgccgcctcgccaaggaggagggcctcctcgtcggcggctcctgcggcatggccgtcgtggccgccctggaggtcgccagggagctcggcccggacgacgtcgtcgtcgtgctgctgccggactccggccgcggctacatgtcgaagatcttcagcgacgagtggatggccggccacggcttcctggaggacacctcctcggccaccgtcgcggacgtcctgcgccacaaggagggcggcaccatgccctccctggtccacatgcacccggacgagaccgtcggccaggccatcgaggtgctccgcgagtacggcgtctcgcagatgccgatcgtgaagccgggcgcgggccaccccgacgtgatggccgccgaggtcgtcggatcggtcgtcgagcgtgagctgctcgacgccctcttcacccagcgcgcctccctggaggactcgctggagagccacatgagcgcgccgctgccgcaggtcggctcgggcgagccggtcgccgccctgatgtccgtcctcggcgaggccgccgacgcggcgatcgtgctggtcgagggcaagccgaccggtgtggtgagccgtcaggacctgctggccttcctggccaacggcggcgccaagtag

SEQ ID NO: 36 - protein, 351 aa, Streptomyces venezuelae
  misc_feature: cystathionine-beta-synthase
MDQHVAEAIGRPDLIRLDDRLLCLRFETMKVVSALAAVRHLLDTGVVRRGDTLLDSSSGIYAYALALACHRHGMRCHIVGSTTVDHTLRTQLAVLGATLEQMEPSSDLKLDQKRRVERIHEILAAHPEYHWMRQYHDDIHYLGYRAIADRVHEETGGARLTVVGGVGSGASTGALTRYLRDRDARTPRGAAGDVDLVGVQPFGSLTFGAQHTSDPEIIIAGIGSSIPFGNVSHELYDTLHWISFDAALAGAVDLLRRHALFAGLSTGAAYLAARHERERRPGRTVLFIAPDTGHRYVDTVFARHDEAAPLGHFTPQDIADQTELTLPWSRMPWNRRSHPPRESTSMESLTT

SEQ ID NO: 37 - DNA, 1056 bp, Streptomyces venezuelae
  misc_feature: Coding sequence of SEQ ID NO 36
atggaccagcacgtcgcggaggcgatcggccggccggacctgatacgcctcgacgaccggctcctgtgtctgcgcttcgagaccatgaaggtggtctcggcactggccgccgtccgtcatctcctcgacaccggtgtcgtacggcgcggggacacgcttctcgacagctccagcggcatctacgcgtacgccctcgccctcgcctgccaccggcacggcatgcgctgccacatcgtcggatcgacgacggtcgaccacaccctgcggacccagctcgccgtactcggggcgaccctggaacagatggaaccgagcagcgacctgaaactcgaccagaaacgacgggtcgagcggatccacgagatcctcgccgcccaccccgagtaccactggatgcggcagtaccacgacgacatccactacctcggctaccgcgcgatcgccgaccgcgtccacgaggagaccggcggcgcccggctcacggtcgtcggcggagtcggctccggcgcctcgaccggcgccctgacccgctacctccgcgaccgggacgccaggacgccacgcggggcggcgggcgacgtcgacctcgtgggcgtccagcccttcggcagcctcaccttcggcgcccagcacacctcggaccccgagatcatcatcgccggcatcggcagctccatccccttcgggaacgtctcccacgagctgtacgacaccctgcactggatctccttcgacgcggcgctggcgggcgccgtcgacctcctgcgccggcacgccctcttcgcgggcctgtcgaccggagcggcctacctcgccgcgcgccacgaacgcgagcgccgccccggacggacggtcctcttcatcgcccccgacaccggccaccgctatgtcgacaccgtcttcgcccgccacgacgaggccgcgcccctagggcacttcacgccccaggacatcgcggaccagaccgaactgaccctgccgtggtcccgcatgccctggaaccgcaggagccatccgccgcgagaatcgacctctatggaatctttgaccacgtaa

SEQ ID NO: 38 - protein, 375 aa, Streptomyces venezuelae
  misc_feature: cystathionine-gamma-lyase
MTMYDVTLRAPAAPAAPGPPSAGAPGPVQRAEDAFAALHGADAALLLPGVVAATTAALLALTNPGGHVIASDQTRDPLRRVLLTELTGARRTVSFVDCTEVDAVAAAVRPETQVVYTPSLSDPLMKLSQLNDVATYAQMNDLLLVVDNTVLSPAQLRPLETGAVVVIEDASPCLDEWGDLSAALVTCVNRYLVPIREQAGLLGGQVDPWAAHLLERSLSTLELRMTARQANAERVADALRGHPAVHTVHRPTFDEADWALETHQGFGALLSFEVVPEAGNLDGVLAAAGPRTAAGSTLTLPALTTHAHLSERLRREAGVSRRLVRVSVGIEDPAPLIRRLRQALDAALAPDAGALAPAASAVSAAPASGAEGESC

SEQ ID NO: 39 - DNA, 1128 bp, Streptomyces venezuelae
  misc_feature: Coding sequence of SEQ ID NO 38
gtgaccatgtacgacgtcaccctgagggccccggccgccccggccgccccgggcccgccgtccgccggggcccccggccccgtccagcgggccgaggacgccttcgccgccctgcacggcgccgacgcggcgctgctgctccccggcgtcgtcgccgccaccacggccgcgctgctcgccctcaccaaccccggcggtcacgtgatcgcctccgaccagacccgcgacccgctgcgccgggtcctcctcaccgagctcaccggcgcccggcggacggtctcgttcgtcgactgcaccgaggtcgacgcggtcgccgcggcggtccgccccgagacccaggtcgtgtacacgccgtccctcagcgacccgctgatgaagctgtcccagctcaacgacgtggccacctacgcgcagatgaacgacctgctcctggtcgtggacaacaccgtcctctcacccgcgcagctacggcccctggagaccggcgccgtcgtcgtcatcgaggacgcctcgccctgcctcgacgagtggggtgacctcagcgccgccctggtgacctgcgtcaaccggtacctcgtaccgatccgcgagcaggccggactcctcggcggccaggtcgacccgtgggcggcgcacctcctggagcggtccctgtccaccctggaactgcggatgacggcccggcaggcgaacgccgaacgggtcgcggacgcgctccgcggccaccccgccgtccacaccgtgcaccggccgacgttcgacgaggcggactgggccctggagacccaccagggcttcggcgcgctgctctccttcgaggtcgtgcccgaggccgggaacctcgacggcgtcctggccgccgccgggccgcgcaccgcggcgggcagcacgctgaccctccccgcgctcaccacccacgcccacctcagcgagcggctgcgcagggaggccggagtctcccggcggctggtgcgcgtctcggtcggcatcgaggaccccgccccgctgatccgccgcctgcgccaggccctggacgcggccctcgcaccggacgccggggccctcgcgcccgccgcgtccgccgtgtccgccgccccggcctccggggccgaaggagagtcatgctga

SEQ ID NO: 40 - protein, 377 aa, Streptomyces venezuelae
  misc_feature: cystathionine-gamma-lyase
MTDMAYGDGTRAVRAGLPDPVKYEPTLPGPVFAAHFHLPGEPTGPYTYGRDENPTWTHLERAIGELEAPGEPDVETLVFASGMAAISAVLLSQLKAGDAVVLPTDGYQALPLLHEQLRAFGIEVRTAPTGGDGQLAVLEGARLLWIETPSNPGLDVCDIRRLVAAAHAAGALVAVDNTLATPLGQRPLELGADFSVASDTKGMTGHGDILLGHVSCRDPRRAAEVRRWRKVVGAIPGPMEAWLAHRSLATLQLRIDRQCSTALALARALAERADVTGLRYPGLPDDPSHTVAARQMRRFGPVVSFELADRKRAERFLEGLRIVDDATSFGGVRSTAERRGRWGGDAVAEGFVRFSVGAEDPEDLIADVLRALDEAGA

SEQ ID NO: 41 - DNA, 1134 bp, Streptomyces venezuelae
  misc_feature: Coding sequence of SEQ ID NO 40
gtgacggacatggcatacggcgacggcacgcgcgcggtacgggccgggcttcccgaccccgtgaagtacgagccgaccctgccgggaccggtcttcgccgcacacttccatctgcccggtgagccgacggggccgtacacctacggccgggacgagaaccccacctggacgcacctggagcgggcgatcggcgagctggaggcgccgggggagccggacgtcgagacgctggtcttcgcctccggcatggccgcgatctccgccgtcctgctctcccagctgaaggccggcgacgcggtggtcctgcccaccgacggctaccaggccctgccgctgctgcacgagcagctccgggccttcggcatcgaggtccgcaccgcgcccaccggcggcgacggtcagctcgccgtgctcgaaggggcccggctgctgtggatcgagaccccgtccaaccccgggctcgacgtctgcgacatccgccgtctggtggccgcggcgcatgccgcaggggcgctcgtcgccgtggacaacaccctggccacaccgctcggccagcgtccgctggaactgggcgcggacttctccgtggccagtgacaccaagggcatgaccgggcacggggacatcctgctgggtcacgtcagctgccgtgatccccggcgggcggcggaggtacggcgctggcgcaaggtcgtcggggccatcccggggcccatggaggcctggctcgcccatcgttcgctcgccacgctccagctgcggatcgaccggcagtgctccaccgcgctcgcgctcgcgcgggcgctcgccgagcgggcggacgtgaccgggctgcgctacccggggctgcccgacgacccctcgcacacggtggccgcccggcagatgcggcgcttcgggccggtggtctccttcgagctggcggaccggaagcgggccgagcggttcctcgaaggactgcggatcgtggacgacgccaccagcttcggcggggtgcgctccaccgcggagcggcgcgggcgctggggcggggacgcggtcgcggagggcttcgtccggttctcggtcggggcggaggacccggaggacctgatcgccgatgtgctgcgggccctcgacgaggccggcgcgtag

SEQ ID NO: 42 - protein, 386 aa, Streptomyces venezuelae
  misc_feature: cystathionine-beta-lyase
MYDFDRETDRRGTWSVQWDGIADRFGVSDLLPFTISDMDFASPPEVLTALRDRVDHGVFGYTDWRLGDFREAIRHWYADRFDTAIDPDSLVYAPSVLSQLSQLLQMWTAPGDGVVVHTPTYDGFRKAVTGLGRELRGVPLGDLEALERELARPDSTMLLLCSPHNPTGRVWTEEELSAFAALAERHGAAVVSDEIHADLTTEGHRHVPWTRIAEPGRGHRWALVTSGTKAFNFPALSGSYGLIGDPDERTAFIRRMETGEGLASPAVLSLTAHIAAYRQGAAWLDELRGYVAGTMETVRRRLADGLPGVRWEPPQAGYLAWIDLRPLGIEETRLQEELITVEKVAIMPGGVYGTPGFVRLNVGCPRAKAERGVDALVRAAARLRTT

SEQ ID NO: 43 - DNA, 1161 bp, Streptomyces venezuelae
  misc_feature: Coding sequence of SEQ ID NO 42
atgtacgacttcgaccgggagacggaccggcgcgggacctggtcggtccagtgggacgggatcgcggaccgcttcggcgtgtccgacctgctccccttcaccatctccgacatggacttcgcctccccgcccgaggtcctcacggccctccgcgaccgggtggaccacggtgtcttcggctacacggactggcggctcggcgacttccgggaggcgatacgccactggtacgccgaccgcttcgacaccgcgatcgacccggactccctggtgtacgcgccctccgtgctcagccagctctcccagctgctccagatgtggaccgcccccggtgacggcgtggtcgtccacacccccacgtacgacggcttccgcaaggccgtcaccgggctcgggcgcgagctgcgcggcgtaccgctcggggacctcgaagccctcgaacgggagctggcccggcccgacagcacgatgctgctgctctgctccccgcacaacccgaccggccgggtgtggaccgaggaggagctcagcgccttcgccgcactggccgagcggcacggcgcggccgtcgtcagcgacgagatccacgccgacctgaccaccgagggccaccggcacgtcccgtggacccggatcgccgagccgggccggggccaccgctgggccctggtcacctcgggcaccaaggccttcaactttcccgcgctctccggctcgtacgggctgatcggcgaccccgacgagcggacggccttcatccggcggatggagaccggcgagggcctggcctcgcccgccgtgctctcgctgaccgcccacatcgccgcgtaccggcagggcgcggcctggctggacgagctgcgcggctatgtggcgggcacgatggagacggtccggcggcgactggccgacgggctgcccggggtgcgctgggagcccccgcaggccggctatctcgcctggatcgacctgcggccgctcggcatcgaggagacccggctccaggaggagctgatcacggtcgagaaggtcgcgatcatgcccggcggggtctacggcacccccggtttcgtccggctgaacgtcggctgcccccgggcgaaggccgaacgcggcgtggacgcgctggtccgcgcggcggcgcgactgcgcacgacctga

SEQ ID NO: 44 - protein, 384 aa, Streptomyces venezuelae
  misc_feature: cystathionine-gamma-synthase
MSDQHSHQSFETRAIHAGNTADPLTGAVVPPIYQVSTYKQDGVGGLRGGYEYSRSANPTRTALEENLAALEGGRRGLAFASGLAAEDCLLRTLLVPGDHVVIPNDAYGGTFRLFAKVVERWGVDFSVANTSDVDAVRAAVNDRTKLIWVETPSNPLLGITDIEAIAGVARQAGVKLVVDNTFASPYLQQPLALGADVVVHSLTKYMGGHSDVVGGALVTADEALGEELAYHQNAMGAVAGPFDSWIVLRGIKTLAVRMDRHSENAEKIVEMLTQHPKVTQVLYPGLPEHPGHEVAAKQMKSFGGMISFRVQGGEQAAVEVCNRAQLFTLGESLGGVESLIEHPGRMTHASVAGSALEVPADLVRLSVGIENVDDLLADLRQALG

SEQ ID NO: 45 - DNA, 1155 bp, Streptomyces venezuelae
  misc_feature: Coding sequence of SEQ ID NO 44
atgagcgaccagcactcccaccagagcttcgagacccgcgcgatccacgcgggcaacaccgccgacccgctgaccggcgcggtcgtcccgcccatctaccaggtgtccacctacaagcaggacggtgtgggcgggctgcgcggcggttacgagtacagccgcagcgccaaccccacccgtaccgccctcgaggagaacctggcggccctggaaggcggccgccgcggtctcgccttcgcctccggcctggccgccgaggactgcctgctgcgcacgctcctcgtgcccggcgaccacgtggtcatccccaacgacgcctacggcggcacgttccggctcttcgccaaggtcgtggagcgctggggcgtggacttctcggtggcgaacacctccgacgtcgacgcggtgcgcgccgccgtgaacgaccgcacgaagctgatctgggtcgagaccccgtcgaacccgctgctcggcatcaccgacatcgaggcgatcgccggcgtggcccgccaggccggggtgaagctcgtcgtcgacaacaccttcgcgtcgccgtacctccagcagccgctggcgctcggcgcggacgtcgtcgtgcactcgctcaccaagtacatgggcggccactcggacgtcgtcggcggcgccctggtcacggccgacgaggcgctcggcgaggagctcgcctaccaccagaacgcgatgggcgcggtcgccggcccgttcgactcgtggatcgtgctgcgcggcatcaagaccctcgccgtgcgcatggaccgccacagcgagaacgccgagaagatcgtcgagatgctcacccagcacccgaaggtcacccaggtcctctacccgggcctcccggagcacccggggcacgaggtcgccgccaagcagatgaagtccttcggcggcatgatctccttccgcgtgcagggcggcgagcaggccgccgtcgaggtctgcaaccgcgcgcagctgttcaccctcggtgagtcgctgggcggcgtcgagtcgctcatcgagcacccgggccggatgacccacgccagcgtcgcgggctccgccctggaggtccccgccgacctcgtccgcctgtcggtgggcatcgagaacgtcgatgacctgctggcggacctgcgccaggcgctgggctag

SEQ ID NO: 46 - protein, 197 aa, Streptomyces venezuelae
  misc_feature: adenosylhomocysteine nucleosidase
MTPMELLGEITADRPLLVLAVKEEAQYLDTALPVLLTGMGKVNAATSLATVLGRGPRPSGIVNLGTAGALRPGWTGTHVVGTVLQHDLDGELLATLTGETYGAPLSLPDGGDVVLATGDAFISDEASRARLAERAPLVDMEGYALAAAAELAGVPLRIVKHVSDEAGDGAARTWRDAVAECARALADWAAENTPYRS

SEQ ID NO: 47 - DNA, 594 bp, Streptomyces venezuelae
  misc_feature: Coding sequence of SEQ ID NO 46
gtgacccccatggaacttttgggagagatcacggccgaccggcccctgctcgtcctcgcggtcaaggaggaggcgcagtacctggacacggcgcttccggtgctgctgaccggtatgggcaaggtgaacgcggcgacctcgctcgcgaccgtcctcggccgcggcccgcggccgtccgggatcgtcaacctggggacggccggggcgctgcgcccgggctggaccgggacgcacgtcgtcggcaccgtgctccagcacgacctcgacggcgagctgctcgccacactgacgggcgagacctacggggcgccgctgtcgctcccggacggcggtgacgtggtgctcgccaccggcgacgcgttcatctcggacgaggcttcccgtgcccggctcgccgagcgggcgccgctggtcgacatggaggggtacgcgctcgcggcggccgcggaactcgccggggtgccgctgcgcatcgtgaagcacgtcagcgacgaggccggcgacggcgccgcgcgcacctggcgtgacgcggtcgccgagtgcgcgcgggcccttgccgactgggcggcggagaacaccccgtaccgctcctga

SEQ ID NO: 48 - protein, 380 aa, Helicobacter pylori
  misc_feature: cystathionine-beta-lyase
MRMQTKLIHGGISEDATTGAVSVPIYQTSTYRQDAIGRHKGYEYSRSGNPTRFALEELIADLEGGVKGFAFASGLAGIHAVFSLLQSGDHVLLGDDVYGGTFRLFNQVLVKNGLSCTIIDTSDISQIKKAIKPNTKALYLETPSNPLLKITDLAQCASVAKDHGLLTIVDNTFATPYYQNPLLLGADIVAHSGTKYLGGHSDVVAGLVTTNNEALAQEIAFFQNAIGGVLGPQDSWLLQRGIKTLGLRMEAHQKNALCVAEFLEKHPKVERVYYPGLPTHPNYELAKKQMRGFSGMLSFTLKNDSEAVAFVESLKLFILGESLGGVESLVGIPAFMTHACIPKTQREAAGIRDGLVRLSVGIEHEQDLLEDLEQAFAKIG

SEQ ID NO: 49 - DNA, 1143 bp, Helicobacter pylori
  misc_feature: Coding sequence of SEQ ID NO 48
atgcgcatgcaaaccaaattaatccatgggggcattagtgaggacgcaacaacgggggcggtgagcgtgcctatttatcaaacttccacctaccgccaagacgccataggccgccataagggctatgaatactctcgctcaggcaaccccacgcgctttgctttagaagaactcatcgctgatttagaagggggggttaaggggtttgcttttgcctctggattagctggaatccacgccgttttttccctcttgcaatcaggcgatcatgtgttattgggcgatgatgtttatggggggactttccgcttgtttaatcaagtgcttgtcaaaaacgggctttcttgcaccattatagacactagcgatatatcccaaattaaaaaggctatcaagcccaacaccaaagccctttatttagaaacccctagtaaccccttgcttaaaatcacggatttagcgcaatgcgctagtgtcgctaaagatcatggtttgctcactatcgtggataacacctttgccaccccctattatcaaaacccgcttcttttgggagcggacattgtggcacatagcggcaccaaatacttaggcgggcatagcgatgtggtcgccgggcttgtaaccactaataatgaagcgctagcccaagagatcgcttttttccaaaacgctatcggtggggttttaggccctcaagacagctggctgttgcaaagagggattaaaacgctgggattgcgcatggaagcccatcaaaaaaacgctctttgtgtggctgagtttttagaaaaacaccctaaagtggaaagggtttattacccgggccttcccactcaccctaattacgaactagctaaaaaacagatgcgtggctttagcgggatgctctctttcactctcaaaaatgatagcgaggcggttgcttttgtagaaagccttaaactattcattttaggcgagagtttgggcggggtggaaagtttggtggggattccggcatttatgacccatgcgtgcatccctaaaacgcaacgagaagctgctgggattagagatggcctggtgcgcttgtctgtagggattgagcatgaacaggatttgttagaagatttagagcaagcgttcgctaaaataggctaa

SEQ ID NO: 50 - protein, 535 aa, Aspergillus nidulans
  misc_feature: cystathionine-beta-synthase
MSSKTTPTVPPVALDSITQHIGNTPLVRLNKLPQNLGINATVYAKLEYFNAGGSVKDRIALRMIEEAERSGRIKPGDTLIEPTSGNTGIGLALVAAVKGYKTIITLPEKMSAEKVSVLRALNATIIRTPNEAAYDSPESHIGVAKRLEKELPNAHILDQYGNENNPLAHELGTAQEIWSQTKGQIKAIVAGAGTGGTITGLSRGLKKHNSNVQVIAADPQGSILALPAALNEEHANEPYKVEGIGYDFIPQVLDQHAVDKWYKTDDKESFQYARRLIAEEGLLVGGSSGSAIAALVKAARDNMFKEGDVVVVILPDSIRSYLTKIQFADDDWLAANDLLPSLPTEVILQSSNKQTQGQDDAFAGSKVSSLRLKPVTTVHSNVPCETAIEVMRDKGFDQLPVLAPSGRKLVGLVTLGNVLSRLTHNRATGRSPVADVMFDFRTISEVVTDPRDMGLASLEPNQNGRDAPKPQTRSRKFVEITMDTSLSVLNRFFEWNSAAIVTERDEGGTLRPVAVVTKVDLLTWMLHHKKNGASE

SEQ ID NO: 51 - DNA, 1608 bp, Aspergillus nidulans
  misc_feature: Coding sequence of SEQ ID NO 50
  misc_feature: cDNA
atgtcttccaaaacgacacccaccgtcccgccggtggctctcgattctatcacgcaacatattggcaatactcctcttgtgaggttgaacaaactaccgcagaacctcgggataaatgcgacggtatatgcaaagctagaatacttcaatgcaggcggaagcgtcaaagatcggatagctcttcgcatgattgaggaagccgaacggtcaggccgtatcaagccaggcgacactctgattgaacctaccagtggtaacactggtattgggttagctctcgtcgcggccgtcaaaggctacaagaccatcatcacccttccagagaagatgtctgctgagaaggtttctgttttacgggctcttaacgcgaccatcatccggactcctaatgaagctgcatacgactcgcccgaatcccacatcggcgttgcgaagcgtcttgaaaaggagctaccaaatgcgcacatcctagatcaatatggtaacgaaaacaatccattggctcatgagctcggcactgcgcaagaaatctggagtcagactaaaggacaaattaaggcgatcgttgctggcgcgggtacgggtggcacgattacagggctctcgcgcggcctgaagaagcacaattccaacgttcaagtaattgcggcggatccccaagggtcaatactggctctcccagctgcactaaacgaggagcatgccaatgagccatacaaggtcgaaggtattgggtatgacttcattccacaggtgctagatcagcacgccgtcgacaagtggtacaagacggacgacaaagaatccttccagtatgctcgccgtttaatagctgaggagggccttctcgttggtgggagcagcggaagcgcaatcgcggccctggtaaaggccgctagggacaacatgtttaaagagggtgatgttgtcgttgtgattttgccagacagcatcagaagctacctcacaaagatacagttcgccgacgacgattggcttgccgccaacgatctcctaccttcgttaccgactgaagtcatcctccaatcctccaataagcagactcaaggacaagacgacgcttttgccggtagtaaggttagctcgctccggctcaaaccagtgacaaccgtccactccaacgtcccttgtgagactgccattgaagtcatgcgcgacaaagggttcgatcaacttcccgttctggcaccatcaggcagaaagcttgtcggcctggttacactcggaaacgttctcagtcgattgacgcacaaccgcgcaactggtaggagccccgttgccgacgtaatgtttgatttccgcaccatttccgaggtcgtcaccgatcctcgagacatgggtcttgcgagcttggagcccaatcagaacgggagggatgcccccaaaccacaaaccaggagcaggaaattcgtcgagattactatggatacatctttaagtgtactgaatcgcttttttgagtggaatagtgcggctattgttacggaaagagatgaaggtgggactctaaggcccgtggctgttgtcacgaaagttgatcttcttacctggatgctccatcataagaagaacggcgcatcggaatag

SEQ ID NO: 52 - protein, 410 aa, Aspergillus nidulans
  misc_feature: cystathionine-gamma-lyase
MTMMAGHPDLPSNGQNGDSNTHQQRQFATLAVHAGAPHDPTTGAVIAPESVGKPVGLYEYTRSSNPNRDNFEEAVASLEHAKYALAFSSGSATTATILHSLAPGSHVVSVSDVYGGTHRYFTKVAAAHGVNVSFSSCLELDVEKLIRPNETKLVWIETPSNPTLALVDIRKVAAVAHRHGVLVVVDNTFMSPYVQNPLDHGADVVIHSVTKYINGHSDVLMGVAAFNSDELKERFTFLQNAIGAVPSPFDCWLAHRGLKTLHLRAREATANATAVALALESSPHVISVNYPGLNSHPNREIAVKQHRKGMGGGMLSFRIKGGHKAAHLFCEYTKIFTLAESLGGVESLCEVPSSMTHAGIPKEEREAAGVYDDLVRMSCGIEDVEDLTADTMQALERAVAASQALENGSA

SEQ ID NO: 53 - DNA, 1233 bp, Aspergillus nidulans
  misc_feature: Coding sequence of SEQ ID NO 52
  misc_feature: cDNA
atgacgatgatggcgggacatccagatctcccctccaacggccagaacggcgactcgaacacacatcagcagcgccaatttgcgactctggccgtccatgctggagctcctcacgatcccaccactggagctgttatcgcaccggaaagtgttggtaagccggtagggctgtacgaatacactcgaagctcgaatcccaatcgagacaattttgaagaggcggttgcttcgctcgagcacgcgaaatatgcactagcattctcctccggatctgcgacgacggcaaccattctccactcgttagctcctggctcgcatgtcgtttccgtctcagatgtatatggaggaacacacagatatttcaccaaggttgccgcggcacatggcgtcaatgtgtcattctcctcgtgcttggaattggacgtggagaagctgatccggccaaacgagactaaacttgtctggattgagactccttcgaaccctaccctagcgctggttgatatccgcaaagttgccgcggttgcgcatcgccatggcgttctggttgtggtcgataataccttcatgagcccttacgttcagaatccattggatcacggtgctgatgtggtgattcactccgttacgaagtacattaacggccattccgatgttctgatgggtgttgcagccttcaattcggacgaattgaaagagcgctttacgttcctccagaatgccattggggctgtaccatctccattcgattgctggctggctcaccgtggtctcaaaacactgcatttgcgtgcgcgagaagccacagccaacgccacggctgttgctctagcactcgaatcttcacctcacgtcatatctgtgaattaccctggactcaactctcatccgaaccgtgaaatcgccgtcaagcagcatcgcaagggcatgggaggcggcatgctgagtttccggatcaagggaggtcacaaggccgcccatctgttctgtgaatataccaagatcttcacacttgcagagagcttaggtggtgtagagagtctctgcgaagttccttcaagcatgacccatgctggaattcccaaagaagagcgagaagctgctggtgtttacgatgacttggtccgcatgagctgcggaattgaagatgttgaggacctgacggctgatacaatgcaggcacttgagagggctgtggctgcaagccaggcgctggagaacggaagtgcttga

SEQ ID NO: 54 - protein, 458 aa, Aspergillus nidulans
  misc_feature: cystathionine-beta-lyase
MSASSTLKKAFPQVDAEGHNLPPSPAPSSPHGSRRYNIATELVYTESNDQYNASSVPIYQSATFKQSSHEGGGEYDYTRSGNPTRTHLERHLAKIMSAQRALVVSSGMAALDVITRLLRPGDEVVTGDDLYGGTNRLLKYLSTNGGIIVHHVDTTNPDKVKEVLTDKTAMVLLETPTNPLIKIVDIPTIAAASHEANPNCLVIVDNTMMSPLLLSPLELGADVVYESGTKYLSGHHDVMAGVIAVNDPALGERLYFPINASGCGLSPFDSWLLMRGVKTLKVRMDQQQANAQRIAEFLESHGFKVRYPGLRSHPQYDLHHSMARGAGAVLSFETGDVSVSERIVANAKLWAISVSFGCVNSLISLPCRMSHASIDAKTRKERAMPEDLIRLCVGIEDVDDLIDDLRRAVSVSTRNRVFWVMLIFSSWYKLALSTSLWTALKRTRRSLRSQVMKEYGYG

SEQ ID NO: 55 - DNA, 1377 bp, Aspergillus nidulans
  misc_feature: Coding sequence of SEQ ID NO 54
  misc_feature: cDNA
atgtcggcttccagtacactgaaaaaagcattccctcaggtcgacgctgagggccataatctgcccccttcacctgccccttcgagtcctcatggcagcaggcgctacaacatcgcaactgagcttgtctacaccgagagcaatgaccaatacaatgccagcagtgtcccaatttaccagagcgcaacattcaagcagtcatcccacgagggaggaggcgagtatgactatacccggtccggcaacccgactcgaacgcatctcgaacgacacctagccaagataatgtcagcgcagcgcgctctcgtggtgtcttccggaatggcggccctggatgtgatcacccgtcttctccgacccggcgacgaggtggttactggtgatgatctgtacggtggaaccaaccgtctactcaaatacctctcaaccaacggcggcattatcgtgcaccacgtcgacactaccaaccccgacaaagtgaaggaagtcttgaccgacaagacagccatggtgctcctggaaaccccgaccaacccgcttatcaagattgtcgatatccctacgattgccgcggcgtcgcacgaagcgaaccctaactgtctagtcatcgtggacaacaccatgatgtctccgctcttgctcagcccgctcgaactcggcgcggacgttgtctacgagagcggcacgaaatacctgtccggccaccacgatgtcatggccggagttattgcagtcaacgacccggccctcggcgagaggctctactttccgatcaacgcgtccgggtgcggactgtcgcccttcgactcttggctgctcatgcgcggagtcaaaactctcaaggtccgcatggatcagcagcaggccaacgcacagcggatcgcagaattcctggagtcgcacggtttcaaggttcggtatccaggtctgcgctcgcacccccagtacgacctgcaccactccatggcccgaggagcaggcgccgtgctttcatttgagacaggcgatgtctctgtcagtgagcgcattgtcgcgaacgcgaagctgtgggctatcagcgttagctttggctgtgtcaacagcttgatcagtcttccctgccggatgagccatgccagtatcgatgcaaagacgcgaaaggagcgtgccatgccggaggatctgattagattgtgtgtgggcattgaagacgtagacgaccttattgatgacttgaggcgagcagtaagtgtttctacgcgtaatcgtgttttctgggttatgctaatattctctagctggtacaagctggcgctgtcaacgtcactctggacggcattgaagcgaacacgccgcagtctgcgtagtcaagtaatgaaggaatatggttatggatga

SEQ ID NO: 56 - protein, 594 aa, Aspergillus nidulans
  misc_feature: cystathionine-gamma-synthase
MLQEPGGPVPPNTDHAVSVSLPTWEANVAYEEGELWLVNKMQCGYPRFFVHPIIQELAQEIVARFGNPEYEATTLFPSPNTARVCHTFILSRISREEAHKIRVLDFVPTKELETELSTIQSLLSCVIYPKEHAKIAKQAWQHTGDGVSSRRGEFCLGALKDGLLTERKNVTGDATRQIIHKGPRRYQGKGSINGIGKVSSHNAVSANKPHEGRESVQFIEERFGRNLSTSLASKAKIAVRRRIAGVLTADVELNEALEKASQEGRVAGLSESDVFLFPSGMSSIFNSHRLLLEARGSMKSVCFGFPYIDTLKILEKWGPGCVFYGRGSSEDLNDLEARLAGGERFLALFTEFPGNPLLKTPDLKRIRALANKYEFAVVVDETVGNFLNINVLSHADIVVSSLTKIFSGDSNVMGGSAVLNPHGRYYPLLKDTFNRGYEDNLWVEDAVFLERNSRDFVSRIEKINHTTEVITAMLQESPIVKNVYYPKFSPTLPFYEAFRTPNGGYGGLFSVTFHSTAEAIAFFDNLEVHKGPSLGTNFTLSSPYTLLAHYSELDWASSFGVEFDLVRISVGLEDVPLLRAKVQQALDAVAKISK

SEQ ID NO: 57 - DNA, 1785 bp, Aspergillus nidulans
  misc_feature: Coding sequence of SEQ ID NO 56
  misc_feature: cDNA
atgcttcaggaacccggaggcccggtacccccaaacacagatcatgctgtgagcgtgtcacttcccacatgggaagcgaacgtggcgtacgaagagggggagctatggcttgtaaataagatgcaatgcggttatccccgattctttgtccatcccatcatccaggagctcgcccaagaaattgttgctcgatttgggaatccagaatatgaggcaacgacccttttcccttctccgaacactgcgcgagtgtgtcacacattcatcctatcaagaatttcacgcgaagaagcacataaaattcgcgtccttgactttgttcctaccaaggagctggaaaccgagctgagcaccattcagtcacttctatcgtgcgttatatatcctaaggagcacgcaaaaatcgcgaaacaagcgtggcaacatacgggtgacggcgtttcaagcaggcggggtgaattctgtcttggtgctctgaaggatggtcttcttacggaaaggaaaaatgttacaggagatgcaacgcgccaaatcatccacaagggccctcggagatatcagggaaagggttcaataaacgggataggtaaagtttcatcacataacgcagtttcagccaacaaaccgcacgaaggacgtgagtcagttcagtttattgaagaacggtttggaaggaacttgagtacctcactagccagcaaggcaaagattgccgtacgcaggcgaatagcaggtgtgctgacggcagacgtggaattgaatgaggctttggagaaagcctcacaggaaggaagagtagcagggctttcagaatcggatgttttccttttcccaagcggcatgagttcgatcttcaattcccacaggttgctcctcgaagcgaggggttctatgaaaagcgtctgcttcggattcccctacattgatacattgaaaatccttgagaaatggggccccggatgtgtcttttacgggcgagggtcatctgaggatctaaatgatctggaggcacggttagccggtggagagagattcttggctctcttcactgaattcccgggcaatccccttctcaagacacctgatcttaagcggatccgagccttagccaacaagtatgagtttgcggtggtcgtcgatgaaacagtgggaaattttctcaacatcaacgtgctttctcacgcggacatcgtggtcagtagtttgacgaaaatcttcagcggagacagcaatgttatgggagggagtgcggtgctaaatccgcacggtcgatactatccattattgaaagatacattcaaccggggctatgaagacaacttatgggtagaagatgccgtcttcctcgaaaggaacagccgcgacttcgtttctcgcattgaaaaaatcaatcatacaacagaggtgataacagcaatgctccaagaatctccaattgttaagaatgtgtattaccctaagttcagtcctacactgcctttctacgaggcatttcgcactccaaatggaggctatggtggtttgttctctgtcactttccactccacagcagaggcgattgccttcttcgacaacctagaagtccacaagggacccagtcttgggactaacttcacgctgagttctccgtatacgctacttgctcactatagtgagcttgactgggcaagctcatttggcgtagagtttgacttggtgcggataagcgtcggtttggaagatgtcccattgcttcgcgctaaagtgcaacaagcacttgatgcagtcgctaagatatctaaatag

SEQ ID NO: 58 - protein, 359 aa, Leishmania major
  misc_feature: cystathionine-beta-synthase
MASANPRNQILANALEAVGNTPCIRLNRVPQKHGIRCEVVAKCEFFNPGGSVKDRIGKQMVLDAEKNGTLKPGSVIVEATSGNTGIGLSMAAAIRGYHMVITMPKKMSHEKETTLQSLGAEVIRTETSLPYDHPDSLIGVARRLRDEKGYVLLDQYTNPSNPGAHYEFTGQEIYDQCGSKVDMVVICAGTGGTITGVAKKLKELIPGVIVVGVDPVGSIIADPEHPGEPVMYHVEGIGYDFVPDVCERKYVDRWVKTRDQESFDLALELHREEGLLVGGSSGSAMAGVLEAAKDLRPDQRCVVLMADGIRNYMGKFADVNWMIEHGFRQGEVTRPTYDTLKKELEEVKAKLAKYESGTQ

SEQ ID NO: 59 - DNA, 1080 bp, Leishmania major
  misc_feature: Coding sequence of SEQ ID NO 58
atggcatccgcaaacccccgcaaccaaatcctggccaacgccctcgaggcggtcggcaacaccccttgcatccgcttgaaccgcgtcccacagaagcatggcattcggtgtgaggtcgtggccaagtgcgaattcttcaaccctggcggcagcgtgaaggatcgcatcggcaagcagatggtactcgacgccgaaaagaacgggacgctcaagccgggatccgtgattgtggaggctacaagtggcaacactggcatcggtctctccatggcggcggcgatccgcggctaccacatggtgatcacgatgccgaagaagatgtcgcacgagaaggagacgacgctacagtcccttggcgccgaggtgatccgcacggagacgtctcttccgtacgaccacccggacagcctcatcggggtcgctcgccgcctgcgcgatgagaagggctatgtgctgctggaccagtacacgaaccccagcaacccgggtgcgcactacgagtttactggccaggagatctacgaccagtgtggcagtaaggtcgacatggtagtcatctgcgccggaaccggtggcacgatcactggagtggccaagaagctgaaggaactcatccccggagtcatcgttgtcggtgtagacccggtcggcagcatcatcgccgatcccgaacaccccggcgagcctgtcatgtaccacgtagagggaatcggctatgactttgtgccagatgtgtgcgagcgtaagtacgtcgatcgctgggtcaagacgcgcgatcaggagagtttcgacctcgccctcgagctgcaccgcgaggagggtctgctggtcggcggtagcagcggctccgcgatggccggcgtgttggaggcagccaaggaccttcgcccggatcagcgctgcgtcgtgctcatggcggacggcatccgcaactacatggggaagtttgccgatgtcaactggatgatcgagcacggcttccggcagggtgaggtgacccgccccacctacgacacgctgaagaaggagctggaggaggtcaaggcgaagctggccaagtacgagagcggcactcagtag

SEQ ID NO: 60 - protein, 459 aa, Leishmania major
  misc_feature: cystathionine-beta-lyase
MMHAISAAKTTINRTILTALEEERQYLEQAMLPVSYKFPKGADYSELPTYPKRPHFDQAAKHPSTQVVIFDGCPNDPYHPTSMPIYQTATFVQPDIEEFGPYDYSRSGNPTRTAIETLVANLEGAHAAFAFSSGMAALQTLVTTLNAGDTILASSDLYGGMHRLLTQITSCLGMTVVFVPMWDTEAVRQALVDNPAAKLLHMESPTNPLMRIVDIRAVCEAAHAHNVKVSIDNTMMSPLRCTPLALGCDYSIHSATKFLCGHSDTMCGLICVKSEEDVKRVAFLQNAQGNALAPFDCWLLLRGIRTLSIRVEKQELNAVAVALFLSRQSHVVSRLHYAGLNPATSPQISSLTEENFYLHRSQTSGPGSVLSIETGSVKRSNAFVRACKLFKLTVSFGSCNSLVEMPCLLSHASIPKEKRTLPVDLIRLSVGIEQIEDILADLTQAIAAAVAIHDNDGSC

SEQ ID NO: 61 - DNA, 1380 bp, Leishmania major
  misc_feature: Coding sequence of SEQ ID NO 60
atgatgcacgcgatcagcgccgccaaaaccaccatcaatcgcactattctgacggcgctggaggaggagcggcagtacctggagcaggctatgcttcccgtgagctacaagttcccgaaaggagctgattacagcgaactccccacctacccaaagcgcccgcacttcgaccaggcagcgaagcaccccagcacgcaggttgtcatcttcgacggctgccccaacgacccttatcacccgacctcgatgcccatctaccaaaccgcgacctttgtgcagcccgacatcgaggagttcggtccgtacgactacagccgtagcggcaacccgactcgcacggccatcgagaccctcgtcgccaacctcgagggtgcccacgccgccttcgctttttcgtccggcatggcggcactgcagacgctcgtgacaacgctgaacgctggcgacaccatcctcgccagtagtgacctttacgggggcatgcaccgtctcctgacacaaatcacctcgtgcctcgggatgaccgttgtgtttgtgccgatgtgggacactgaggcggtgcgccaagcgctcgtcgataacccagcggcgaagctgctgcacatggagagccccacgaacccactgatgcgcattgtcgacatccgtgctgtctgtgaggctgcccacgcgcacaacgtaaaggtgtccatcgacaacactatgatgtctccgctccgctgcaccccgctcgccctgggctgcgactactccatccacagcgccaccaagtttctgtgcggccacagcgacaccatgtgcggtcttatctgcgtgaagtctgaggaggatgtgaagcgggtggcctttctgcagaacgcgcagggaaacgccctcgccccgtttgactgctggctgctcctgcgtggcatccgcacgctgtccattcgcgtcgagaagcaggagctgaacgccgtcgcggtggcgctgttcctgtcgcgtcagagccacgtcgtgtcgcgcctgcactacgctggtctgaaccccgcaacgtccccccagatctcttccttaactgaggagaacttctacctgcaccgctcgcagacctccggccctggcagcgtgctctccattgagaccggcagcgtcaagcgctccaacgcgtttgtacgggcctgcaagctgttcaagctcaccgtcagctttggaagctgcaactcgctggttgaaatgccctgcctcctctcgcacgcatccatcccgaaggagaagcgcacacttccagttgacctgatccgactgtccgtgggtatcgaacagatcgaggatatacttgcggacctaacgcaggctatcgccgccgcggtcgccatccatgacaatgacgggtcctgctga

SEQ ID NO: 62 - protein, 399 aa, Leishmania major
  misc_feature: cystathionine-beta-lyase
MPNQSFEECSVETKLGHVGRNPDEQWGFINCPIYRGSTVLYKSINDALKLRSVYWYGTAGNPTVTNLQNAWTALTGGAGSVVVSSGMQAIAYALLATANSGDNILVADAVYLPTRNLCDHFLVTKGITTTYYDPCITADELRELLHQKPNTTVIFMESPGSQTFEIQDVPMICAVAREFNVTTIIDNTWATPIFFDALGIGCDISVEAGTKYVGGHSDLLLGLISANEKWYPKLRGFMSLLQSVPSNEDCYLGLRGLRTMYIRLKEAEQRGLEMARFMESRKEVLKVLHPAFPSCRGHEIWRRDFTGSSGLFSIVLRPEYALQDVERMIDGYKLFSIGFSWGGFESLVMCFDCTKFRTATTFAPGGILLRFQIGLEGLEDLKQDLCQGFDKLHAESMGC

SEQ ID NO: 63 - DNA, 1200 bp, Leishmania major
  misc_feature: Coding sequence of SEQ ID NO 62
atgcccaatcagtcatttgaggaatgttctgtcgagaccaagctgggccatgtcggccgcaacccggatgagcaatggggcttcatcaactgccccatctaccgcggctccaccgtcctctacaagagcatcaatgacgcactgaagctccgctccgtgtactggtacggcactgcgggcaaccccaccgtgacgaatctgcagaacgcctggactgcgctcaccggcggtgctggcagtgttgtcgtgtcgtctggcatgcaggcgatcgcttacgcgctgctggcgaccgcgaacagcggtgacaatattcttgtggcggacgcagtctacctgcccacccgtaacctgtgcgaccacttcttggtgaccaagggcatcaccacgacctactacgatccctgcatcacggcagacgagctgcgtgagctgctccaccagaagcccaacaccaccgtaatcttcatggagtcaccggggtcgcagaccttcgaaatccaggacgtgccgatgatctgcgctgtggcacgcgaattcaacgtcacgacgatcatcgacaatacgtgggccacccccatcttttttgacgctctcggcatagggtgcgacatctcggtggaggctggcaccaagtacgtcggcggccactcagacctgctgctgggtcttatctcagccaacgagaagtggtacccaaagctgcggggcttcatgagcctcctgcagtcggtgcccagcaacgaggactgctacctgggcctgcgggggcttcgcaccatgtacattcgccttaaggaggcggagcagcgcgggctcgagatggcgcgcttcatggagagccgcaaagaggtactcaaggtcctgcacccggcgttccccagctgtcgtggccacgagatctggaggcgcgacttcaccggctcctccggcctcttctcgatcgtgctgcgccccgagtacgcgctgcaggatgtggagcggatgatcgacggctacaagctcttcagcataggcttttcatggggcggcttcgagagtcttgtgatgtgcttcgactgcaccaagttccgcacggcaaccaccttcgcccctggtggcatcctcctccgtttccagatcggcctcgagggcttggaggatctcaagcaggacctctgccagggtttcgacaagctgcacgcggagtcgatgggctgctaa

SEQ ID NO: 64 - protein, 384 aa, Oryza sativa
  misc_feature: cystathionine-beta-lyase
MKEPSVATILTSFENSFDGFGSMSTPLYQTATFKQPSATDNGPYDYTRSGNPTRDVLQSLMAKLEKADQAFCFTSGMAALAAVTHLLKSGQEIVAGEDIYGGSDRLLSQVAPRHGIVVKRIDTTKISEVTSAIGPLTKLVWLESPTNPRLQITDIKKIAEIAHYHGALVLVDNSIMSPVLSRPLELGADIVMHSATKFIAGHSDLMAGILAVKGESSLAKEIAFLQNAEGSGLAPFDCWLCLRGIKTMALRVEKQQANAQKIAEFLASHPRVKKVNYAGLPDHPGRSLHYSQAKGAGSVLSFLTGSLALSKHVVETTKYFNVTVSFGSVKSLISLPCFMSHASIPSAVREERGLTDDLVRISVGIEDADDLIADLDHALRSGPA

SEQ ID NO: 65 - DNA, 1155 bp, Oryza sativa
  misc_feature: Coding sequence of SEQ ID NO 64
  misc_feature: cDNA
atgaaggagcctagcgtggcgacgatactgacgagcttcgagaactcgttcgatgggttcgggtctatgagcacgccgctgtaccagacggccacgtttaagcagccttcagcaaccgataatggaccttatgattacactagaagtggtaaccctacacgtgatgttctccaaagccttatggctaagcttgagaaggcggatcaggcattctgcttcaccagtgggatggcagcactagctgcagtaacacacctccttaagtctggacaagaaatagttgctggagaggacatatatggtggctcagaccgtctgctctcacaagttgccccgagacatgggattgtagtaaaacgaattgatacaaccaaaattagtgaggtaacttctgcaattgggcccttgactaaactagtatggcttgaaagtcccaccaatccccgtctacaaattactgatataaagaaaatagcagagatagctcattaccatggtgctcttgttttagtagacaacagcatcatgtctcctgtgctctcccgtcctctagaacttggagcagatattgttatgcactcagcaaccaaatttatagctggacatagcgatcttatggctggaattcttgcggtgaagggtgaaagcagcttggctaaagagattgcatttctacaaaatgctgaaggatcaggtttggcaccatttgattgctggctttgtttgagaggaatcaaaaccatggctttgcgggtggagaagcagcaggctaatgctcagaagattgctgaatttctagcttctcatccaagagtaaagaaagtgaactatgcaggacttcctgatcatcctggacgatctctacactattcccaggcaaagggagcgggttcagttctcagtttcctaactggttcattagctctctcaaaacatgttgttgagaccacaaagtacttcaatgtaacagttagctttggaagtgtgaaatcgctcattagcctgccatgcttcatgtcacacgccagcatcccttctgcggttcgcgaggagcgcggcctgacagacgatctagtcaggatatcggttggaattgaggatgccgacgacctcatagcggatcttgatcatgctctccggtctggtccagcttag

SEQ ID NO: 66 - protein, 476 aa, Oryza sativa
  misc_feature: cystathionine-beta-lyase
MSAAAAAAAAAAIPTSLGRLFHLRPTPNPSRNLSGSSAQPLLRLSYHPRLTLSRRMEAPAAIADSHGGGDLSASAVGAEALGAVAAPDFDVEMKEPSVATILTSFENSFDGFGSMSTPLYQTATFKQPSATDNGPYDYTRSGNPTRDVLQSLMAKLEKADQAFCFTSGMAALAAVTHLLKSGQEIVAGEDIYGGSDRLLSQVAPRHGIVVKRIDTTKISEVTSAIGPLTKLVWLESPTNPRLQITDIKKIAEIAHYHGALVLVDNSIMSPVLSRPLELGADIVMHSATKFIAGHSDLMAGILAVKGESSLAKEIAFLQNAEGSGLAPFDCWLCLRGIKTMALRVEKQQANAQKIAEFLASHPRVKKVNYAGLPDHPGRSLHYSQAKGAGSVLSFLTGSLALSKHVVETTKYFNVTVSFGSVKSLISLPCFMSHASIPSAVREERGLTDDLVRISVGIEDADDLIADLDHALRSGPA

SEQ ID NO: 67 - DNA, 1431 bp, Oryza sativa
  misc_feature: Coding sequence of SEQ ID NO 66
  misc_feature: cDNA
atgagcgccgccgccgccgccgccgccgccgccgcaatccccacctctctcggccgcctcttccacctccgccccaccccgaacccctcccggaaccttagcggcagctcagcgcaacccctcctccgcctcagctaccacccacgcctcacgctctctcgccgcatggaggcgccggcggcgatcgccgactcccacggcggcggcgacctgagcgcgtccgcggtcggcgcggaggcgctgggcgccgtcgccgctccggatttcgatgtggagatgaaggagcctagcgtggcgacgatactgacgagcttcgagaactcgttcgatgggttcgggtctatgagcacgccgctgtaccagacggccacgtttaagcagccttcagcaaccgataatggaccttatgattacactagaagtggtaaccctacacgtgatgttctccaaagccttatggctaagcttgagaaggcggatcaggcattctgcttcaccagtgggatggcagcactagctgcagtaacacacctccttaagtctggacaagaaatagttgctggagaggacatatatggtggctcagaccgtctgctctcacaagttgccccgagacatgggattgtagtaaaacgaattgatacaaccaaaattagtgaggtaacttctgcaattgggcccttgactaaactagtatggcttgaaagtcccaccaatccccgtctacaaattactgatataaagaaaatagcagagatagctcattaccatggtgctcttgttttagtagacaacagcatcatgtctcctgtgctctcccgtcctctagaacttggagcagatattgttatgcactcagcaaccaaatttatagctggacatagcgatcttatggctggaattcttgcggtgaagggtgaaagcagcttggctaaagagattgcatttctacaaaatgctgaaggatcaggtttggcaccatttgattgctggctttgtttgagaggaatcaaaaccatggctttgcgggtggagaagcagcaggctaatgctcagaagattgctgaatttctagcttctcatccaagagtaaagaaagtgaactatgcaggacttcctgatcatcctggacgatctctacactattcccaggcaaagggagcgggttcagttctcagtttcctaactggttcattagctctctcaaaacatgttgttgagaccacaaagtacttcaatgtaacagttagctttggaagtgtgaaatcgctcattagcctgccatgcttcatgtcacacgccagcatcccttctgcggttcgcgaggagcgcggcctgacagacgatctagtcaggatatcggttggaattgaggatgccgacgacctcatagcggatcttgatcatgctctccggtctggtccagcttag

SEQ ID NO: 68 - protein, 514 aa, Oryza sativa
  misc_feature: cystathionine-gamma-synthase
MATVSSLPSPAFLAADPAAALPSATILRFPPNFVRQLSTKARRNCSNIGVAQIVAAAWSDPARPASLPGGGAGGRRGASSRAAATPAAAAAASAAAEATAEVGAIPNAKLGQPSAAALAEQALLGSDASLAVHAGERLGRRIATDAITTPVVNTSAYWFNNSQELIDFKEGRHASFEYGRYGNPTTEALEKKMSALEKAESTVFVASGMYASVAMLSALVPAGGHVVTTTDCYRKTRIYMETELPKRGITMTVIRPADMDALQNALDNNNVSLFFTETPTNPFLRCIDIDLVSKMCHSKGALLCIDSTFASPINQKALTLGADLVIHSATKYIAGHNDVIGGCISGRDELVSKVRIYHHVVGGVLNPNAAYLILRGMKTLHLRVQCQNNTAMRMAQFLEEHPKIARVYYPGLPSHPEHHIAKSQMTGFGGVISFEVAGDFDATRRFIDSVKIPYHAPSFGGCESIIDQPAIMSYWDSKEQREIYGIKDNLIRFSIGVEDFEDLKNDVVQALDKI

SEQ ID NO: 69 - DNA, 1545 bp, Oryza sativa
  misc_feature: Coding sequence of SEQ ID NO 68
  misc_feature: cDNA
atggccaccgtctcctccctcccgtccccggcgttcctggccgccgacccggccgccgcgctcccctccgccaccatcctccgcttcccgccaaacttcgtccgccagctcagcaccaaggcccgccgcaactgcagcaacatcggcgtcgcgcagatcgtcgccgccgcctggtccgaccccgcgcgccccgcctccctccccggcggcggcgccggcggccgtcgcggcgcctcctcccgcgccgccgccacgcccgccgccgccgccgccgcctccgcggcggcggaggccacggccgaggttggcgccatccccaacgccaagctcggccagccgtccgccgccgcattggccgagcaggccctgctcggttcagacgccagcctcgccgtccacgcgggcgagaggctgggcagaaggatcgccacagacgcgatcaccacgccagtagtgaacacctcggcctactggttcaacaactcgcaggagctcattgacttcaaggaggggaggcatgctagcttcgagtatgggaggtatggtaacccgaccactgaggcattggagaagaagatgagtgcactggagaaagccgagtccactgtctttgtggcatccgggatgtatgcaagtgtggctatgctcagcgcgcttgttccagcaggtgggcacgttgtgaccaccactgattgttaccgcaagacgagaatttacatggagactgaactacctaagaggggaattacgatgaccgttattagacctgctgacatggatgctctccaaaatgcactggataataataatgtatccctattcttcaccgagactcctactaatccatttctgagatgcatcgacattgatcttgtctcaaaaatgtgccatagcaagggagcattgctttgtattgatagtacctttgcatccccaatcaatcagaaggcactaactctaggtgctgacttagttatccattcagcaacaaagtacattgctgggcacaatgatgttattggaggctgcatcagtggcagagatgagctagtttccaaagtccgcatctatcaccatgtagttggtggtgttctaaatccgaatgctgcctacttgattcttcgaggcatgaagacactgcatctccgtgtacaatgtcagaataatactgcaatgcggatggcccaatttttagaggaacatccaaagattgcacgtgtctactatcctggtctgccaagtcacccagaacatcacattgccaagagtcagatgaccggctttggtggtgtcattagttttgaggttgctggagactttgatgctactaggagatttattgattctgttaagataccctatcatgccccatcatttgggggctgtgagagcattattgatcagcctgccatcatgtcttactgggattcaaaggagcagagagaaatctatggaatcaaggacaacttgatcaggttcagcattggagtggaggattttgaggatttgaagaatgacgttgttcaggcgcttgacaagatctaa

SEQ ID NO: 70 - protein, 472 aa, Oryza sativa
  misc_feature: cystathionine-gamma-synthase
MAKSSSLVTKVLVQRRRRVHRSRRSPTKPERAAAADTFGSLAGCPSSPEYPVVPDLDDCVVDTAVDDAPVVAAEAAGRRASDETLAVHAGEKLGSGAAETDSIATPIVSGTTHWFRDSADLIAFREGWRRSFEYGRYGNPTVKVLEEKISALERAEATLVTSSGMNAIVATLLALVPPGGHVVVTADCYSEARAFIHDKLSKMGITSTFVDLDDDMEALESVLDEDEVTMFYADSMTNPHLKVVDVARVAELCHRRGALVCIDSTLASPINQKPLALGADVVLHSATKYIAGHHDVIAGCVSGSEALISRIRAWHHDLGGAISPNAAYMIIRGLKTLALRVEAQNRTALRMARLLEKHPKIERVYYPGLESSPWHGVATRQMAGAGGVVSFEVASDMRGVMRFVDALELPLIATSLGGCESLVQQPAVMSYWGKSEEEKAKNGIKDNFVRFSFGIEKFEDLRDDILQALEKI

SEQ ID NO: 71 - DNA, 1419 bp, Oryza sativa
  misc_feature: Coding sequence of SEQ ID NO 70
  misc_feature: cDNA
atggcgaagtcatcgtctctcgtgacgaaggttttggtgcagcggcggcggcgcgttcaccgttctcgccgttctcccaccaagccggagcgcgctgccgccgccgacacgttcggttccctcgcgggttgtccctcgtcgccggagtatccggtggtgccggacttggacgactgcgtcgtcgacaccgccgtggatgacgctcccgtggtggcggcggaggcggcaggccgacgcgcgtcggacgagacgctggcggtgcacgccggggagaagctcgggagcggcgccgctgagacggactcgatcgcgacgccgatcgtgagcggcacgacgcactggttcagggactcggcggacctcatcgccttccgggaaggctggcggcggagcttcgagtacgggcggtacggaaacccgacggtgaaggtgctggaggagaagatcagcgcgctggagagggcggaggcgacgctggtgacctcctccggcatgaacgccatcgtcgccacgctgctcgccctcgtcccccccggcggccacgtcgtcgtcaccgccgactgctacagcgaggcccgcgccttcatccacgacaagctatccaagatgggcatcacgtcgacgttcgtggatcttgacgacgacatggaggcgctggaatccgttcttgatgaggacgaggtgacgatgttctacgccgactcgatgacgaacccacacctgaaggtcgtcgacgtggcacgcgtggcggagctgtgccaccggaggggcgcgctggtgtgcatcgacagcacgctggcgtcgcccatcaaccagaagccgctcgccctcggagccgacgtggtcctccactccgccaccaagtacatcgccggccaccatgacgtcatcgccggctgtgtcagcggctcagaggccctcatctcgaggatacgcgcgtggcaccacgacctcggcggcgccatcagtccgaacgcggcgtacatgatcattcggggattgaagacgttggcgctgcgggtggaggcgcagaacaggacggcgctgcggatggcgcggctgctggagaagcaccccaagatcgagcgcgtctactaccctgggctggagagcagcccgtggcacggcgtcgcgacgaggcagatggccggcgccggcggcgtggtcagcttcgaggtggcgtcggacatgcgcggcgtgatgaggttcgtggacgcgctggagttgccgctcatcgcgacgtcgctcggcggctgcgagagccttgtccagcagccggccgtcatgtcctactggggtaagagtgaggaggagaaggcgaagaatgggatcaaggacaactttgtgaggttcagttttgggatcgagaagtttgaggatctcagggatgatatactccaagccctagagaagatttaa

SEQ ID NO: 72 - protein, 486 aa, Oryza sativa
  misc_feature: cystathionine-gamma-synthase
MAKSSSLVTKVSAQRRRRVHRPRRSPTKPERSAAAVALSHEKILLAGVDAAADMFGSLAGCPSSPEYPVVPDMDDRYVDGAVDDAPAAAAGRRASDETLAVHAGEKLGSGAAETDSIATPIVSGTTHWFRDSADLIAFREGRRRSFEYGRYGNPTVKVLEEKISALERAEATLVTSSGMNAIVATLLALVPPGGHVVATADCYSEARAFIRDKLSNMGITSTFVDLDDDMEALECVLDESEVTMFYADSMTNPHLKVVDVTRVAELCHRRGALVCIDSTLASPINQKPLALGADVVLHSATKYIAGHHDVIAGCVSGSEALISRIRAWHHDLGGAISPNAAYMIIRGLKTIALRVEAQNRTALRMARLLEKHPKIERVYYPGLESSLWHGVATRQMAGAGGVVSFDVASDLRGVMRFVDALELPLIATSLGGCESLVQQPAVMSYWGKSEEEKAKNGIKDNFVRFSFGIEKFEDLRDDILQALEKI

SEQ ID NO: 73 - DNA, 1461 bp, Oryza sativa
  misc_feature: Coding sequence of SEQ ID NO 72
  misc_feature: cDNA
atggcgaagtcatcgtccctcgtgacgaaggtttcggcgcagcggcggcggcgagtgcaccgtcctcgccgttctccgaccaagccggagcgctctgccgccgccgtggcgctgagccacgagaagattcttctcgccggcgttgatgcagccgccgacatgttcggttccctcgcgggttgtccctcgtcgccggagtatccggtggtgccggacatggacgaccgctacgtcgacggcgccgtggatgacgctccggcggcggcggcgggccgacgcgcgtcggacgagacgctggcggtgcacgccggggagaagctcgggagcggcgccgctgagacggactcgatcgcgacgccgatcgtgagcggcacgacgcactggttcagggactcggcggacctcatcgcgttccgggaaggccggcggcggagcttcgagtacgggcggtacggcaacccgacggtgaaggtgctggaggagaagatcagcgcgctggagagggcggaggcgacgctggtgacctcctccggtatgaacgccatcgtcgccacgctgctcgccctcgtccctcccggcggccacgtcgtcgccaccgccgactgctacagcgaggcccgcgcattcatccgcgacaagctctccaacatgggcatcacgtcgacgttcgtcgatcttgacgacgacatggaggcgctggaatgtgttcttgatgagagcgaggtgacgatgttctacgccgactcgatgacgaaccctcacctgaaggtcgtcgacgtgacacgcgtggcggagctgtgccaccggagaggcgcgctggtgtgcatcgacagcacgctggcatcgcccatcaaccagaagccgctcgccctcggcgccgacgtggtcctccactccgccaccaagtacatcgccggccaccacgacgtcatcgccggctgcgtcagcggctcggaggccctcatctcgcggatccgcgcgtggcaccacgacctcggcggcgccatcagtccgaacgcggcgtacatgatcattcggggattgaagacgattgcgctgcgggtggaggcgcagaacaggacggcgctgcggatggcgcggctgctggagaagcaccccaagatcgagcgcgtctactaccctgggctggagagcagcctgtggcacggcgtcgcgacgaggcagatggccggcgccggcggcgtggtcagcttcgatgtggcgtcggacttgcgcggcgtgatgaggttcgtggatgcgctggagttgcctctcatcgcaacgtcgctcggtggatgtgaaagccttgtgcagcaaccggccgtcatgtcctactggggtaagagtgaggaggagaaggcgaagaatgggatcaaggataattttgtgaggttcagttttgggattgagaagtttgaggatctcagggacgacatactccaggccctagagaagatttag

SEQ ID NO: 74 - protein, 507 aa, Saccharomyces cerevisiae
  misc_feature: cystathionine-beta-synthase
MTKSEQQADSRHNVIDLVGNTPLIALKKLPKALGIKPQIYAKLELYNPGGSIKDRIAKSMVEEAEASGRIHPSRSTLIEPTSGNTGIGLALIGAIKGYRTIITLPEKMSNEKVSVLKALGAEIIRTPTAAAWDSPESHIGVAKKLEKEIPGAVILDQYNNMMNPEAHYFGTGREIQRQLEDLNLFDNLRAVVAGAGTGGTISGISKYLKEQNDKIQIVGADPFGSILAQPENLNKTDITDYKVEGIGYDFVPQVLDRKLIDVWYKTDDKPSFKYARQLISNEGVLVGGSSGSAFTAVVKYCEDHPELTEDDVIVAIFPDSIRSYLTKFVDDEWLKKNNLWDDDVLARFDSSKLEASTTKYADVFGNATVKDLHLKPVVSVKETAKVTDVIKILKDNGFDQLPVLTEDGKLSGLVTLSELLRKLSINNSNNDNTIKGKYLDFKKLNNFNDVSSYNENKSGKKKFIKFDENSKLSDLNRFFEKNSSAVITDGLKPIHIVTKMDLLSYLA

SEQ ID NO: 75 - DNA, 1524 bp, Saccharomyces cerevisiae
  misc_feature: Coding sequence of SEQ ID NO 74
atgactaaatctgagcagcaagccgattcaagacataacgttatcgacttagttggtaacaccccattgatcgcactgaaaaaattgcctaaggctttgggtatcaaaccacaaatttatgctaagctggaactatacaatccaggtggttccatcaaagacagaattgccaagtctatggtggaagaagctgaagcttccggtagaattcatccttccagatctactctgatcgaacctacttctggtaacaccggtatcggtctagctttaatcggcgccatcaaaggttacagaactatcatcaccttgccggaaaaaatgtctaacgagaaagtttctgtcctaaaggctctgggtgctgaaatcatcagaactccaactgctgctgcctgggattctccagaatcacatattggtgttgctaagaagttggaaaaagagattcctggtgctgttatacttgaccaatataacaatatgatgaacccagaagctcattactttggtactggtcgcgaaatccaaagacagctagaagacttgaatttatttgataatctacgcgctgttgttgctggtgctggtactggtgggactattagcggtatttccaagtacttgaaagaacagaatgataagatccaaatcgttggtgctgacccattcggttcaattttagcccaacctgaaaacttgaataagactgatatcactgactacaaagttgagggtattggttatgattttgttcctcaggttttggacagaaaattaattgatgtttggtataagacagacgacaagccttctttcaaatacgccagacaattgatttctaacgaaggtgtcttggtgggtggttcttccggttctgccttcactgcggttgtgaaatactgtgaagaccaccctgaactgactgaagatgatgtcattgttgccatattcccagattccatcaggtcgtacctaaccaaattcgtcgatgacgaatggttgaaaaagaacaatttgtgggatgatgacgtgttggcccgttttgactcttcaaagctggaggcttcgacgacaaaatacgctgatgtgtttggtaacgctactgtaaaggatcttcacttgaaaccggttgtttccgttaaggaaaccgctaaggtcactgatgttatcaagatattaaaagacaatggctttgaccaattgcctgtgttgactgaagacggcaagttgtctggtttagttactctctctgagcttctaagaaaactatcaatcaataattcaaacaacgacaacactataaagggtaaatacttggacttcaagaaattaaacaatttcaatgatgtttcctcttacaacgaaaataaatccggtaagaagaagtttattaaattcgatgaaaactcaaagctatctgacttgaatcgtttctttgaaaaaaactcatctgccgttatcactgatggcttgaaaccaatccatatcgttactaagatggatttactgagctacttagcataa

SEQ ID NO: 76 - protein, 394 aa, Saccharomyces cerevisiae
  misc_feature: cystathionine-gamma-lyase
MTLQESDKFATKAIHAGEHVDVHGSVIEPISLSTTFKQSSPANPIGTYEYSRSQNPNRENLERAVAALENAQYGLAFSSGSATTATILQSLPQGSHAVSIGDVYGGTHRYFTKVANAHGVETSFTNDLLNDLPQLIKENTKLVWIETPTNPTLKVTDIQKVADLIKKHAAGQDVILVVDNTFLSPYISNPLNFGADIVVHSATKYINGHSDVVLGVLATNNKPLYERLQFLQNAIGAIPSPFDAWLTHRGLKTLHLRVRQAALSANKIAEFLAADKENVVAVNYPGLKTHPNYDVVLKQHRDALGGGMISFRIKGGAEAASKFASSTRLFTLAESLGGIESLLEVPAVMTHGGIPKEAREASGVFDDLVRISVGIEDTDDLLEDIKQALKQATN

SEQ ID NO: 77 - DNA, 1185 bp, Saccharomyces cerevisiae
  misc_feature: Coding sequence of SEQ ID NO 76
atgactctacaagaatctgataaatttgctaccaaggccattcatgccggtgaacatgtggacgttcacggttccgtgatcgaacccatttctttgtccaccactttcaaacaatcttctccagctaaccctatcggtacttacgaatactccagatctcaaaatcctaacagagagaacttggaaagagcagttgccgctttagagaacgctcaatacgggttggctttctcctctggttctgccaccaccgccacaatcttgcaatcgcttcctcagggctcccatgcggtctctatcggtgatgtgtacggtggtacccacagatacttcaccaaagtcgccaacgctcacggtgtggaaacctccttcactaacgatttgttgaacgatctacctcaattgataaaggaaaacaccaaattggtctggatcgaaaccccaaccaacccaactttgaaggtcaccgacatccaaaaggtggcagaccttatcaagaagcacgctgccggccaagacgtgatcttggttgtcgacaacaccttcttgtccccatatatctccaatccattgaacttcggtgcagacatcgttgtccactccgctacaaagtacatcaacggtcactcagacgttgtgctcggtgtcctggccactaataacaagccattgtacgagcgtctgcagttcttacaaaacgccattggtgctatcccatctcctttcgatgcttggttgacccacagaggtttgaagactttgcatctacgtgtcagacaagctgccctcagcgccaacaaaatcgctgaattcttggcagcagacaaggaaaacgttgtcgcagtcaactacccaggtttgaagacacaccctaactacgacgtagtgttaaagcaacaccgtgatgcccttggtggtggtatgatctccttcagaatcaagggtggtgctgaagctgcttccaagttcgcctcctccacaagactgttcacattggccgaatcccttggtggtatcgaatctctattggaagtgcccgctgtgatgacccacggtggtatcccaaaggaggccagagaggcctctggtgtttttgacgacttggttagaatctctgtcggtattgaagacactgacgatcttttggaagacatcaagcaagccttgaaacaagccaccaactaa

SEQ ID NO: 78 - protein, 340 aa, Saccharomyces cerevisiae
  misc_feature: cystathionine-beta-lyase
MIDRTELSKFGITTQLSVIGRNPDEQSGFVNPPLYKGSTIILKKLSDLEQRKGRFYGTAGSPTIDNLENAWTHLTGGAGTVLSASGLGSISLALLALSKAGDHILMTDSVYVPTRMLCDGLLAKFGVETDYYDPSIGKDIEKLVKPNTTVIFLESPGSGTMEVQDIPALVSVAKKHGIKTILDNTWATPLFFDAHAHGIDISVEAGTKYLGGHSDLLIGLASANEECWPLLRSTYDAMAMLPGAEDCQLALRGMRTLHLRLKEVERKALDLAAWLGNRDEVEKVLHPAFEDCPGHEYWVRDYKGSSGLFSIVLKNGFTRAGLEKMVEGMKVLQLGFSWGG

SEQ ID NO: 79 - DNA, 1023 bp, Saccharomyces cerevisiae
  misc_feature: Coding sequence of SEQ ID NO 78
atgattgatcgtaccgagttatcgaagtttggtattactacgcaactgtctgttattggacgtaatccagatgaacaaagtggctttgttaatccacctttgtataaggggtcaaccatcattcttaaaaaacttagtgatttagaacaaaggaaaggaagattttacgggacagcaggttctccaactattgacaatttagaaaatgcctggacgcatttaaccggcggtgctgggacagtgctatcagcttctgggcttggttctatctctttggcgctattggccctttcgaaagctggtgatcatatcttgatgactgatagtgtctacgtgccaacacgtatgctatgtgatggtttattggccaagttcggtgttgaaacggattattatgacccatcaatagggaaggatatagaaaaactagttaagccaaatacaaccgtcattttcctcgaaagcccgggttctgggaccatggaagtacaggatattccagctttggtctctgttgccaaaaagcatgggataaagacaattctagacaacacatgggcaacgccactcttttttgatgctcatgcgcatggtatcgatatttcggtagaagctgggacaaaatatttgggtggtcattcagatcttcttattggtctggcctccgcaaatgaagaatgttggccgctattacggtcaacttatgatgcaatggcaatgttaccaggtgccgaggactgtcaattagcattgcgaggaatgcgtacattgcacttaagattgaaagaggtagaaagaaaagccctggatttggctgcttggctcggaaatcgagatgaggttgaaaaagtgcttcaccccgcctttgaagattgtcccggacatgaatactgggttcgtgactacaaaggttcttcaggcttattttccattgtccttaaaaatgggttcacaagagctggtctggagaaaatggtagaagggatgaaagttttgcaattgggattttcatggggtggctag

SEQ ID NO: 80 - protein, 465 aa, Saccharomyces cerevisiae
  misc_feature: cystathionine-beta-lyase
MPIKRLDTVVVNTGSQNDQHSASVPPVYLSTTFKVDLNNEDAQNYDYSRSGNPTRSVLQHQIGKLYRVPQENVLAVSSGMTALDVILRGLVLLNGTDNHTPTIIAGDDLYGGTQRLLNFFKQQSHAVSVHVDTSDFEKFKTVFQSLDKVDCVLLESPTNPLCKVVDIPRILRFVKCISPDTTVVVDNTMMSGLNCNPLQLNPGCDVVYESATKYLNGHHDLMGGVIISKTPEIASKLYFVINSTGAGLSPMDSWLLVRGLKTLGVRLYQQQRNAMILAHWLENSCGFKPTRTNKATKTRFVGLRSNPDFKLHKSFNNGPGAVLSFETGSFEHSKRLVSSKKLSIWAVTVSFGCVNSLLSMPCKMSHASIDPELRKERDFPEDLVRLCCGIENIVDLKKDLLAAMVDADIIEVRENGKYLFNKLNKNLAVNTTIDDLHKPLSIYEEFYNQDLIRKDSELNIKSSKL

SEQ ID NO: 81 - DNA, 1398 bp, Saccharomyces cerevisiae
  misc_feature: Coding sequence of SEQ ID NO 80
atgccgatcaagagattagatacagttgtggtaaataccggctctcaaaatgaccaacattcagcctccgtgccaccggtgtatttgtcgactaccttcaaagtggacttgaataatgaagatgcacagaactacgattattccagatcgggaaacccgaccagaagtgtccttcaacaccagattggtaagctttatcgtgtcccacaggaaaacgtattagctgtgagcagtggtatgacggcgctagacgtcatcctgcgtgggctcgtcttacttaacggcactgacaaccatacgccaacaataatagccggcgatgatctttatggaggcacccaaaggctgctgaattttttcaagcaacagagtcatgcagtctctgttcatgtggacacttccgattttgaaaagttcaaaaccgttttccagtctttagataaagttgattgtgttcttctagagtctccgaccaatccgctttgcaaggttgtagatatccctagaatattacgttttgtgaaatgcatatctcccgacactacagttgtcgttgataatactatgatgagtggactcaattgtaatcctcttcaactgaatccaggctgcgatgtcgtatacgaatctgctaccaagtacttgaatggtcatcacgatttgatggggggtgttattatcagcaaaacaccagaaatagcctcgaagctttactttgtcattaattctacaggagctggattatccccaatggattcttggctacttgtgaggggcctaaaaactctaggagttagattatatcaacagcagagaaatgctatgatattggctcattggctagaaaattcatgcggattcaaacctaccagaacaaacaaggctacgaaaactagatttgttggattacgctccaacccggatttcaagctgcataaatcgttcaataatggcccaggtgccgtgttatccttcgaaacggggtccttcgaacattcaaagagactggtcagttccaaaaaactgagtatatgggctgtgacggtatctttcgggtgtgtaaattcgcttctatctatgccttgcaaaatgtcccatgcttccattgatcccgaattaaggaaagagagagattttcctgaagatttggttcgtctttgctgcggtatcgaaaatatagtagatttgaagaaagatttattagcggcgatggttgacgctgatattatagaagtaagagaaaatggcaaatatcttttcaacaaattgaataagaacctagctgtgaacactaccatcgatgacctgcataagcctttaagtatttacgaagaattttacaatcaagatctcatcagaaaggactcagaattgaatattaagagttcgaaattgtaa

SEQ ID NO: 82 - protein, 639 aa, Saccharomyces cerevisiae
  misc_feature: cystathionine-gamma-synthase
MISRTIGESIPPNTKHAVSVCLPTWEATVGYEEGESSIINSLTTGYPRFFIHKSIKKLCEILSAKYSMEDEACLCFPSYKVANRCREFIKVKTGLSTKVRILQLCTPKPMNQEEKLWRRECKITVVFVDQEIFPVMKQYWQHSGEIVSSRMAEYILHELQVKDNLKKMETVDNGKKFMTEDENRVNEEYIETRFGRNLNFLAADKAKYLIRKRIATKVVEKIDSEGLSDLFSFEHYNESNGPFNVGSGEALDDDQLNSDIPAETITSMGESGSNSTFENTATDDLKFHVNPNTDVYLFPSGMASIFTAHRLLLNFDAKRLSRSSSRQDKLIGYGPPFKKTVMFGFPYTDTLSILRKFNHTHFLGQGDSTSMNALKNILHSGEQILAVFIEAPSNPLLKMGDLQELKRLSDLYSFYIVVDETVGGFVNIDVLPYADIVCSSLTKIFSGDSNVIAGSLVLNPRGKIYEFARKFMKTEDGYEDCLWCEDALCLERNSRDFVERTIKVNTNTDILLKRVLLPQVGKLFKKIYYPSLTSEDTKRNYDSVMSTKDGGYGGLFSLTFFNIEEAKKFFNNLELCKGPSLGTNFTLACPYAIIAHYQELDEVAQYGVETNLVRVSVGLENSDVLCNVFQRAIEKALGE

SEQ ID NO: 83 - DNA, 1920 bp, Saccharomyces cerevisiae
  misc_feature: Coding sequence of SEQ ID NO 82
atgatatctagaaccattggtgaatctattccgcccaacacaaaacatgctgtttccgtgtgtttgcccacatgggaagctacagtaggctatgaagagggtgaaagcagtataattaactcgcttactaccggatatccaagatttttcatacacaagtcaataaagaaattatgtgaaattttatccgctaagtactccatggaagatgaagcttgtctttgcttcccctcttacaaggttgccaataggtgcagagagtttatcaaagtgaaaacgggtctttctaccaaagtacgcatcttacaactatgtacacctaaacctatgaaccaagaggaaaagctctggagaagggaatgtaaaatcacggtggtgtttgtagaccaggaaatattccccgtgatgaagcagtattggcaacattctggtgagattgtatctagcagaatggcggagtatattttacatgaattacaagtcaaggataatcttaagaagatggaaacagtggataacggtaaaaaatttatgaccgaagatgaaaacagagtgaacgaagaatatatagaaacgagatttgggagaaatttaaattttcttgctgctgataaagccaaatatctaatacgaaagaggattgccacaaaagttgttgaaaaaattgactctgaaggtctatcggacctattctcttttgaacactataacgagagcaacggtccattcaatgttggcagtggagaggctctggatgatgatcagttgaattcggacatacctgccgaaacaattacttctatgggagaaagtggctcaaacagtacttttgaaaacacagctaccgatgacttgaagtttcatgtcaaccctaatacggatgtttacttatttccaagtggaatggcgtccattttcactgcccacaggctgcttttaaacttcgatgccaagcgcttatcaaggtcttctagtagacaagataagttgattggatatggacctccgttcaagaagaccgtcatgtttggattcccatatacagatactctgagcattcttcggaagttcaatcacacacatttcttgggacagggagattcaacgtcaatgaacgctctgaaaaatattttacattctggtgagcaaatactggcggtatttatagaagctccatcgaacccactgttgaaaatgggagatttacaagaactgaaaaggctatcagacttgtattcattttacatagtggtcgatgaaactgtgggtggctttgtcaatatagatgtattaccgtatgccgatattgtttgtagttcgctaacgaaaatcttcagcggcgactcgaacgttattgcaggatccttagtactaaatcctagaggaaagatttatgagtttgctagaaaatttatgaagactgaagacggatatgaagattgcctatggtgcgaagatgcgttatgcctggaaagaaattcgagagacttcgtggaacgtacaatcaaggtgaatacaaacactgatatattgttaaaaagggtgttattgcctcaggtgggaaaattgttcaagaaaatatattaccccagtttaacatcagaggataccaagcggaattatgacagcgttatgtctacaaaagatggtgggtatggaggtttattttcactaacttttttcaatattgaagaggcaaagaagtttttcaataatctggaattgtgcaaaggaccttctctcggtacgaatttcactttggcgtgtccatatgctattattgcccactaccaggaattagacgaggtagctcaatatggtgtcgaaacaaatcttgtaagagtgagcgtcggtttggaaaatagcgacgttttatgcaatgtttttcagcgtgccattgagaaagctttaggggaataa

SEQ ID NO: 84 - protein, 575 aa, Saccharomyces cerevisiae
  misc_feature: cystathionine-gamma-synthase
MTEIEFGQPLPSNLDYAVSFGIPTWDSAIGYAEKVPEVIGKMATGYPRYFPQPPVQRLCAYFVKKFGRGSENCRPFPSVNLGLKCFEYVKSVSGPESKAHLEVETVTIKNRGAKTSKEPAELVLTIAAVLASEEEFETVKEYWKLRGECVSSRLALSVNQLLDCANHGSEQVLRELEAGVFAAKKGEEKAKNLIKGRIVENRFRPFGLEKKTPNWEGLNLNPNEDVYLVSSGMSAISTARNLLTFWEEKKNSGDSLNKTTSDQKKKPLLCDTVGIFGFPFKDTQVIMTKFGKCKFFGFGNSRDVVELQKFLETSKQRILAVFVETPSNPLLNMPDLKKLRSLADQYGFFIVIDDTIGGLNVDILPYADIVSTSLTKLFNGASNVMGGSVVLNPKSSLYPYAREYFRSANFEDLLWCEDAIVLERNSRDFEDRTLRANANTGILLNDLLLPEEGKICKKIYYPTVTSKETFENYESVRNERGGYGCLFSVAFFNEGDAKAFYDSLKVFKGPSNGTNFTLACPYVHLAHHSELEEVSKFGADPNIIRVSVGLEDIQWLLKVFSSALDVVKSRGSKHS

SEQ ID NO: 85 - DNA, 1728 bp, Saccharomyces cerevisiae
  misc_feature: Coding sequence of SEQ ID NO 84
atgacggaaattgaatttgggcaaccccttcccagcaatctagactatgctgtttcttttggcattcctacatgggattcggcaataggctatgcggaaaaggtgccagaggtaataggcaaaatggctacaggatatccgaggtactttcctcaaccccccgtccagaggctttgtgcttactttgttaagaaatttgggcgaggttcagaaaactgtcgtccttttccatccgttaacctgggtttaaagtgtttcgagtacgtaaaatctgtctcaggacctgaaagtaaagctcaccttgaagtggaaacagttacgattaaaaaccgtggggcaaaaacgtcgaaagaaccggcggaattggtgctaaccattgctgctgtccttgcatcggaggaagagttcgagactgtgaaagagtattggaaattaagaggtgaatgtgtatctagtcgattggcgttgtctgtcaaccaacttcttgactgcgctaatcatggctcagagcaggtattgcgtgagctagaggctggtgtttttgcagcaaagaagggggaggagaaagcaaagaacctgataaagggaaggatagttgagaatcgcttcaggccctttggtttggagaagaaaaccccgaattgggagggactcaacctcaacccaaatgaagacgtttatcttgtatcaagtggcatgtctgcaatatctacggctcgtaatttactcaccttttgggaggagaagaagaattcaggagattcattaaacaagaccacctctgaccaaaagaaaaagccattattatgtgacactgttggcatcttcggcttccctttcaaagatacgcaggtcattatgactaaatttggaaaatgtaaattttttggatttggaaattctagagacgttgtcgaattacaaaagtttttagaaacaagcaaacaacgcattcttgccgttttcgtcgagacaccttcaaatcctctgctgaatatgccggatttgaaaaaattgaggagtttggccgaccagtatggcttttttattgtaattgatgatactattgggggccttaacgttgacatcttgccctatgctgatatagtcagcacgtccctgactaagcttttcaatggtgctagtaatgtaatgggcggctctgtcgttttaaatccgaaatcatctctgtatccatatgctcgtgagtacttcaggagcgctaatttcgaggatttgttatggtgtgaagatgcgatagttttagaacgtaattccagggattttgaagatagaactttacgtgctaatgcaaacacaggaatattgttgaatgatttgcttttacccgaggaggggaaaatatgcaaaaagatttattatccaactgttacttctaaggagacctttgagaactacgaaagtgtacgtaatgaacgtggtggctatggatgcttgttctctgtagcctttttcaatgagggcgatgcaaaagcattctatgactctttaaaggtattcaagggaccttctaatggtactaactttaccttagcatgtccatatgtccatctggcgcaccactcagaattagaagaagtttccaagtttggggccgaccccaacattattagagtaagcgtaggtttagaagatatccagtggttattgaaagttttttcaagtgctttagatgttgtgaaaagcagaggctccaaacactcatga

SEQ ID NO: 86 - protein, 649 aa, Saccharomyces cerevisiae
  misc_feature: cystathionine-gamma-synthase
MVSAQVATELGQPIPLDTQHAVSVCFPTWKSVISYVEKDPKVLGCLKSGYPRFWIHPSIQKLRDILIEKYAKENETCFCFPSYRVAKRCREYVRRKCAHRNGKVRILQLATAKPINEEQKTWKRECKIAVVFVDGAYENILKQYWQYTGEIISSRLAEYVLHELFMVEKKSSPAEEKEYIEMRYGRNLNFAFADRAKELIKKRIATKVIDKDEHDEEENYHFLAGNQDEQDFQDTFLDSSLNEANHGEDHDGGISGEVDSQEEPHNGLVSTIPPEPIEMSTIEEEQSVEEDAGRCALRVCPERDVFLFPSGMASIFTAHRLLLQWDSLRLNRSRNGSDVTSSPPNKKTVIFGFPYADTLHVLQEFNETYFLGEGDESSMKELTKILHSGEQILAVFIETPSNPLLKMGNLLELKRLSELFGFFIIIDETVGGIVNIDGLPFADIVCSSLTKTFSGDSNVIGGSMVLNPQSRVYEFASRFMQLEDEYEDLLWCEDAIYLERNSRDFIARTIRINYSTEYLLDKILKPHVGENKLFKKIYYPNLTSKETLTNYDMVRCKKEGGYGGLFSLTFHDEDHAAAFYDNLKLNKGPSLGTNFTLAFPYTLMTYYHELDMAEKFGVERNLLRISVGLESQSILGKIFQEAIDKTVEI

SEQ ID NO: 87 - DNA, 1950 bp, Saccharomyces cerevisiae
  misc_feature: Coding sequence of SEQ ID NO 86
atggtttcagcgcaagtggctaccgagctaggacagcctataccactggacactcaacatgcggtatctgtatgtttcccgacatggaaatctgtaatttcgtatgtggagaaagaccccaaagttttagggtgtttaaagtctggctaccccaggttttggatccacccctcgattcaaaagctgcgtgatattttaatagaaaagtatgctaaagaaaacgaaacgtgcttttgcttcccctcttatagagttgccaaacggtgcagagagtatgtaagaaggaagtgcgcgcatcgcaatggcaaagtaaggatattacagttagccacggcgaaacctatcaacgaggagcagaagacgtggaaaagagagtgtaagattgcggtagtgtttgtagatggggcatacgagaatatattaaaacagtattggcaatacaccggagagatcatctcaagcagactagctgaatatgttttgcatgaacttttcatggtagaaaagaaatccagccctgcagaggaaaaggaatacatagagatgcggtatggcaggaatctgaattttgcatttgccgatcgagccaaggaattgattaagaagaggatcgctacaaaggttatagataaagatgagcacgatgaggaggaaaactaccatttcttagcaggtaaccaagatgaacaggacttccaggacacgtttctggattcatccctcaatgaggctaaccacggagaagatcatgacggcgggattagcggtgaagtagacagtcaagaagaaccgcataatggccttgtttccactatacccccggagcccatcgaaatgagcacgatagaggaagagcaatccgtagaggaagatgccgggaggtgtgcattaagggtttgccctgagagagacgtttttttgtttccgagcggcatggcgtcgatattcactgcgcatagattgctgttgcaatgggactcactgcgtttgaatagatccagaaatggaagcgacgtcacctcgtcacctccgaataagaagaccgtgatttttgggttcccttatgcggatacattacatgtcttgcaagagtttaatgaaacgtatttcttgggagaaggagatgagtcttcaatgaaagaactaacaaaaattttacattccggggagcaaattcttgccgtttttatagagaccccatcaaacccactattaaaaatgggaaatttactggaactgaaaaggctttcggaattatttgggtttttcattatcattgatgaaactgtgggtggaatagttaatattgacggtctaccgtttgctgatatcgtttgcagttcattgacgaagacatttagtggggactctaacgtcattggtggttcaatggttttaaatccacaaagccgggtatacgagtttgcctcccgttttatgcaattagaagacgaatatgaagatttattatggtgcgaagacgcaatttacctggaaagaaattccagagatttcattgctagaaccatcaggatcaactacagcaccgaatacctcttagataaaatactgaagccacacgtaggcgaaaataaactattcaagaaaatctactaccctaatctaacctcaaaagaaacactaaccaactatgacatggtgaggtgtaagaaagaaggtggatacggcgggctcttttcactaacgttccatgacgaagatcatgctgcggccttttatgacaatctaaagctgaacaagggtccttccttgggcacaaatttcacactcgcattcccctatacgctcatgacatactaccacgaactcgacatggctgaaaaatttggggttgagagaaatcttctcagaatcagcgtaggcctcgaaagtcagtcaatattgggcaagatcttccaagaggcaattgataaaacggtagaaatatga

SEQ ID NO: 88 - protein, 464 aa, Arabidopsis thaliana
  misc_feature: cystathionine-beta-lyase
MTSSLSLHSSFVPSFADLSDRGLISKNSPTSVSISKVPTWEKKQISNRNSFKLNCVMEKSVDGQTHSTVNNTTDSLNTMNIKEEASVSTLLVNLDNKFDPFDAMSTPLYQTATFKQPSAIENGPYDYTRSGNPTRDALESLLAKLDKADRAFCFTSGMAALSAVTHLIKNGEEIVAGDDVYGGSDRLLSQVVPRSGVVVKRVNTTKLDEVAAAIGPQTKLVWLESPTNPRQQISDIRKISEMAHAQGALVLVDNSIMSPVLSRPLELGADIVMHSATKFIAGHSDVMAGVLAVKGEKLAKEVYFLQNSEGSGLAPFDCWLCLRGIKTMALRIEKQQENARKIAMYLSSHPRVKKVYYAGLPDHPGHHLHFSQAKGAGSVFSFITGSVALSKHLVETTKYFSIAVSFGSVKSLISMPCFMSHASIPAEVREARGLTEDLVRISAGIEDVDDLISDLDIAFKTFPL

SEQ ID NO: 89 - DNA, 1395 bp, Arabidopsis thaliana
  misc_feature: Coding sequence of SEQ ID NO 88
  misc_feature: cDNA
atgacatcttctctgtcacttcactcctccttcgtaccttccttcgctgatctctccgaccgaggtttgatcagtaagaactctccgaccagtgtttccatttccaaggttccaacttgggagaagaagcagatctcaaatcggaactctttcaagctgaattgcgtgatggagaaaagtgtcgatggtcaaactcattctaccgttaacaataccactgatagcttaaatactatgaatatcaaagaagaagctagcgtctctacattattggtgaacttggataataaatttgatccctttgatgcaatgagcactccgctttaccaaactgctacttttaagcagccttctgctattgaaaatggaccttatgattatacaagaagtggcaatcctacacgggatgcattggaaagcctccttgcgaagcttgacaaggcagatagagcattttgctttactagcggaatggctgctcttagtgctgttacacatcttatcaaaaatggcgaagaaattgttgctggagatgatgtatatggtggctctgacagattactatcccaagttgttccaagatctggcgttgtggtaaaacgagtaaacacaactaagttagacgaggttgctgctgcaattggtccccaaacaaagcttgtgtggcttgagtctccaacaaacccaagacaacaaatttctgatatacgaaaaatatctgagatggctcatgctcaaggtgctcttgtgttggtggacaacagtattatgtcaccagtgctctctcggccattagaacttggagctgacatcgtgatgcactcggctactaagtttatagccggacacagtgacgtgatggcaggtgtgcttgctgtaaaaggcgaaaaattggcaaaggaggtgtatttcctccaaaactcagaaggttctggattagctcctttcgactgttggctttgccttcgaggaatcaagacaatggctttacggatagaaaagcaacaggaaaacgcacggaaaattgcaatgtacttgtcttctcatccaagagtaaagaaagtgtactatgctggtctaccagatcatcctggtcaccatctccacttctctcaggcgaagggtgcaggatcagtttttagctttataactggatcagttgcgctttcaaagcatctcgtagaaaccaccaaatacttcagcatagctgtcagttttgggagtgttaagtcactgataagcatgccatgcttcatgtcacatgcaagcatacctgcagaagttcgtgaggccagaggtttgacggaagatcttgtccgtatatctgcaggaattgaagatgttgatgatttgatatctgatcttgacattgccttcaaaaccttccctctctag

SEQ ID NO: 90 - protein, 412 aa, Arabidopsis thaliana
  misc_feature: cystathionine-gamma-synthase
MQILKENASNQRFVTRESEVNRICCGFNGFQLGERLRRDMKTSSITTLVVNTTTYFFKDTTELIDFKEKRIDLYEYARYGNPTTMALEEKISVLEGAESTLVMASGMYASNVMLLALVPTNGHIVATKDCYKETRIFMENFLTKLGITVTFIDSDDIAGLQTLVNNHEVSLFFTESPTNPFLRCVDIKLVSKICHRRGTLVCIDATIATPINQKTLALGADLVHHSATKYIGGHNDFLAGSISGSMELVSKIRNLHKLLGGTLNPNAAYLLIRGMKTMHLRVRQQNSTGMKMAQVLEAHPKVSRVYYLGLPSHPEHLIAKRQMTGIGGLISFEIDGDLKTTIKFIDALKIPYLAASFGGCESLVDQLATGIWDIPREERLKDGFQDNLVRFSFGIEDFEDIKADVLQALETI

SEQ ID NO: 91 - DNA, 1239 bp, Arabidopsis thaliana
  misc_feature: Coding sequence of SEQ ID NO 90
  misc_feature: cDNA
atgcaaatattaaaagaaaatgcaagtaaccaacgtttcgtaaccagagaatcagaggtaaataggatttgttgtgggtttaatggttttcaattaggtgaaagattacgtcgtgatatgaagacgagttcgatcacgacccttgtagtcaacacaacgacctacttcttcaaagatactaccgagctaatcgacttcaaggaaaaacggattgacttgtatgagtatgctcgttatggaaacccaacaactatggcactcgaagaaaagattagtgtacttgaaggggctgaatcaactttggtaatggcatcggggatgtatgcaagtaatgttatgcttttggctttggttcctacaaatggacatattgtggcaactaaagattgttacaaggagacaaggattttcatggagaattttcttactaaactgggaatcactgtaacttttattgactctgatgatattgcggggctccaaactcttgttaataaccatgaggtttctttgttctttaccgagtccccaacaaatccatttcttcgatgtgtagatattaagttagtttcaaaaatctgtcatagaagaggaactcttgtttgtatagacgctaccattgcaacacctatcaatcaaaagacacttgctcttggtgctgatcttgttcaccattctgctactaagtacattggaggccacaacgattttctagctggaagcatatccggttcaatggagttggtttccaagattcgcaatttgcataagcttttgggaggcacgcttaatccgaacgctgcatatttactcatacgaggcatgaagacgatgcatcttcgtgtaagacaacagaattcaaccggtatgaagatggcccaagtgttagaagcacatcccaaggtgagtcgcgtttactatctaggccttccgagtcatcccgaacatttaatagccaagcgacaaatgactggtattggtggcctgatctctttcgagattgatggagatctcaagactacaatcaagttcatcgatgctctgaagatcccttaccttgcagcatcgtttggtggatgcgagagcctcgtggaccaactcgccacagggatctgggatataccacgagaagaaaggcttaaagatggattccaagacaacctagttcgcttcagttttggtattgaagactttgaggacatcaaggctgacgttctccaagctcttgaaaccatctga

SEQ ID NO: 92 - protein, 563 aa, Arabidopsis thaliana
  misc_feature: cystathionine-gamma-synthase
MAVSSFQCPTIFSSSSISGFQCRSDPDLVGSPVGGSSRRRVHASAGISSSFTGDAGLSSRILRFPPNFVRQLSIKARRNCSNIGVAQIVAAKWSNNPSSALPSAAAAAATSSASAVSSAASAAAASSAAAAPVAAAPPVVLKSVDEEVVVAEEGIREKIGSVQLTDSKHSFLSSDGSLTVHAGERLGRGIVTDAITTPVVNTSAYFFKKTAELIDFKEKRSVSFEYGRYGNPTTVVLEDKISALEGAESTLVMASGMCASTVMLLALVPAGGHIVTTTDCYRKTRIFMENFLPKLGITVTVIDPADIAGLEAAVNEFKVSLFFTESPTNPFLRCVDIELVSKICHKRGTLVCIDGTFATPLNQKALALGADLVVHSATKYIGGHNDVLAGCICGSLKLVSEIRNLHHVLGGTLNPNAAYLIIRGMKTLHLRVQQQNSTAFRMAEILEAHPKVSHVYYPGLPSHPEHELAKRQMTGFGGVVSFEIDGDIETTIKFVDSLKIPYIAPSFGGCESIVDQPAIMSYWDLPQEERLKYGIKDNLVRFSFGVEDFEDVKADILQALEAI

SEQ ID NO: 93 - DNA, 1692 bp, Arabidopsis thaliana
  misc_feature: Coding sequence of SEQ ID NO 92
  misc_feature: cDNA
atggccgtctcatcattccagtgccctaccatcttctcctcctcctcaatctccggctttcaatgccgttctgatccagatctcgtcggttctcccgtcggtggatcatctcgccgtcgtgtccatgcctccgccgggatttcttcctcattcaccggggacgctggattatcctccaggatcttaagatttcctcctaatttcgtccgtcagctgagcattaaagcccgtagaaactgtagcaacatcggtgttgcacagatcgtggcggctaagtggtccaacaacccatcctccgcgttaccttcggcggcggcggctgctgctacctcgtctgcatctgcggtttcttccgccgcatctgcagccgcagcctcgtccgccgccgccgcccctgtggctgccgcgcctcccgtcgtgctgaaaagcgtcgatgaggaggttgtggtggccgaggaggggatcagggagaagataggtagtgtacagctgacggattccaaacattctttcttgagctccgatgggagcctcactgttcatgccggtgaaagattaggccgtggtatagtgacggatgctatcactactcctgtagtcaacacatctgcctacttcttcaagaaaactgctgagcttattgacttcaaggagaaaaggagtgtgagtttcgagtatggtcgttatggtaaccctacgactgtggtacttgaagataagataagtgcacttgaaggggctgaatcaactttggttatggcatctgggatgtgtgcaagcactgttatgcttttggcattggttcctgctggtggacacattgtcactactactgattgctacaggaagactaggatcttcatggagaattttcttcccaagttggggatcactgtcactgtgattgatcctgctgatatcgcagggcttgaagctgcagtgaatgagttcaaggtatctctgttcttcactgagtccccgacaaacccattccttagatgtgtcgacattgagctagtttcaaaaatatgccacaagaggggaactctggtttgcattgatggcacctttgcaacacctctgaatcagaaagcccttgctcttggtgctgatcttgtcgtgcactctgctacaaagtacattggaggacacaatgatgttcttgctggatgcatctgtggttcactgaagttggtttcagaaattcgcaatctgcatcatgtgttgggaggaacacttaacccaaacgctgcgtacctaatcatccgaggcatgaagacattgcatcttcgtgtacagcaacagaattcgaccgcttttagaatggccgaaattttagaggcacatcctaaggtgagtcatgtgtactatccaggccttccaagtcatcccgaacatgaactcgccaagcgacaaatgactggttttggaggtgtggtcagtttcgagattgatggagacattgaaacgacaatcaagtttgtggattctctaaagattccttacattgcaccatccttcggtggctgcgaaagcattgttgaccaacctgctatcatgtcctactgggatctgccgcaagaggagagactaaagtatggaatcaaagataacttggttcgtttcagctttggagttgaagactttgaagatgtcaaagctgacattcttcaagctctcgaagccatctga

SEQ ID NO: 94 - protein, 390 aa, Klebsiella variicola
  misc_feature: cystathionine-beta-lyase
MFDFATPIDRHGTWCTQWDYVADRFGAADLLPFTISDMDFATAPCILDAVSQRLAHGVFGYSRWRNEAFLGAIAHWYASRFNSVIDPQTVVYGPSVIYMVAETIRQWSSEGDGVVVHTPAYDAFYKTITANRREIAPVPLTLTNNQWHCDMARLEAVLAEPKNTLLLLCSPHNPTGKVWRREELQTMAALCQKHGVRVISDEIHMDMAWGEHRHIPWSEVAQGPWALFTSGSKSFNIPAFTGAYGFIPEANARDSYLQALKARDGLSSPSVPALVAHIAAYRDGAPWLDALRDYLQANMRYVAATLNNAFPALCWQPPEATYLAWIDLRPLNIDDHALQQALIAEQKVAIMPGYTYGPEGNGFLRLNVGCPREKLERGVEGLIAALRSLS

SEQ ID NO: 95 - DNA, 1173 bp, Klebsiella variicola
  misc_feature: Coding sequence of SEQ ID NO 94
atgtttgattttgccacccccatcgaccgccacggtacctggtgcacacagtgggactatgtcgccgaccggtttggcgccgccgacctgctgccgttcaccatctccgatatggatttcgccaccgcgccctgcattctcgacgccgtcagccagcggctggcccacggggtgttcggctacagtcgctggcggaacgaggcgtttctcggggcgattgcccactggtacgccagccggtttaacagcgtcatcgatccgcaaacggtggtctacgggccgtcggttatctacatggtggcggaaaccatccgccagtggtcgagcgaaggcgacggcgtcgtcgtccatacccctgcctacgatgcattctataagaccatcaccgctaaccgccgggagattgccccggtgccattgaccctgacgaataatcaatggcactgcgatatggcccgcctggaagccgtgctggcggaaccgaagaataccctgctgctgctgtgcagcccgcacaacccaacggggaaagtctggcgtcgcgaggagctccaaaccatggcggccctgtgtcaaaaacatggcgtcagggtgattagcgacgaaattcatatggacatggcctggggcgaacaccgccatatcccgtggagcgaggtcgctcaggggccatgggcgctgttcacctccggttcgaagagctttaatattccggcctttaccggggcctatggctttatccccgaggcgaacgcgcgcgacagctacctgcaggcgctgaaggcccgcgacgggctctcttctccctccgtgccggcgctggtcgcccatatcgccgcctaccgcgacggggctccctggctcgacgccttgcgtgactatctgcaggcgaatatgcgctatgtcgccgctacgcttaacaatgccttccccgcactctgctggcagccgccggaggccacctacctggcgtggatcgacctgcgccccctgaacatcgacgaccacgcgctacagcaggcgctgatcgccgaacagaaggtggcgattatgccgggttatacctatggtcctgagggcaacggctttctgcgcctcaacgtcggctgcccgcgcgagaagctggaacgcggtgtcgaaggactgattgccgccctgcgcagcctcagctga

SEQ ID NO: 96 - protein, 232 aa, Klebsiella variicola
  misc_feature: adenosylhomocysteine nucleosidase
MKIGIIGAMEEEVTLLRDKIENRQTITIGGSEIYTGQLHGVDVALLKSGIGKVAAATGATLLLERCQPDVIINTGSAGGLAPTLKVGDIVVSDEARYHDADVTAFGYDYGQLPGCPAGFKADDKLVAAAEDCIKALDLNAVRGLIVSGDAFINGSVALAKIRHNFPQAIAVEMEATAIAHVCHNFKVPFVVVRAISDVADQQSHLSFEEFLAVAARQSTLMVENLVQNLARG

SEQ ID NO: 97 - DNA, 699 bp, Klebsiella variicola
  misc_feature: Coding sequence of SEQ ID NO 96
atgaaaattggcattattggcgctatggaagaggaagttaccctcctgcgcgacaaaatcgaaaaccgccagactatcactattggcgggagcgaaatctataccggccaacttcacggcgttgacgtcgctctgctgaagtctggtatcgggaaagtggcggcggcgacgggcgcgaccctgctgctggagcgctgccagcctgacgtgatcattaataccggttctgccggcggcctcgcgccgacgctgaaggtcggcgatatcgtcgtctccgatgaagcgcgctaccacgatgccgacgtcaccgccttcggttatgactatggccagttgcctggctgcccggcgggctttaaagcggacgataagctggtggccgccgccgaagactgcattaaagcgttagatctcaatgccgttcgcggcctgatcgtcagcggcgatgccttcattaacggctccgtcgcgctggcgaagatccgccataatttcccgcaggcaatcgccgtggaaatggaagcgaccgccatcgctcacgtctgccataacttcaaggttcccttcgtggtggttcgcgccatctccgacgtggccgatcagcagtcccacctcagttttgaggagttccttgcggtcgccgccagacagtccaccctgatggtggagaatctggtacagaacctggcacgtggctaa

SEQ ID NO: 98 - protein, 398 aa, Lactobacillus plantarum
  misc_feature: cystathionine-beta-lyase
MEQAAFIKKYATTRQHTDSLKWDALQARYGNPDLLAMWVADMEFKVPEQVTQALQERVAHGIYGYSITPDRYYQAFIDWEQARHGLTLKRDWIRFGSGVVNSLYALVNILTQPGDGVLILTPVYYPFFNAVRDNHRQLVTSELRNDRGHYTIDFEDVEQQIKANEVRLFIQCSPHNPVGRIWSEDEETRLLALCEQYHVLVISDEIHQDLEIDRTNHPFKSALTVANGRFSNRVIVVNAPSKTFNLAALLNSHLVIPNPKLRQRYDAQIGRFSQTEISVLGQVAGQTAYESGAAWLDQILAIVRDNYHYLCTTFAAHAPKLVLADLQGTYLTWLDLRAYVQPAATKAFIQDQCQLAVDFGEWFSPDDQGFVRLNLATNPQNVREAAQRIVTCLSQMQK

SEQ ID NO: 99 - DNA, 1197 bp, Lactobacillus plantarum
  misc_feature: Coding sequence of SEQ ID NO 98
atggaacaagcagcatttattaagaagtacgcaacaacgcgccagcatacggattcattgaagtgggacgcactacaagctcggtatggcaatcctgatttgttggcaatgtgggtcgctgacatggaattcaaagtgccagaacaagttacgcaagcactacaagaacgagtggcgcatggcatttatggttattccattacgcctgatcgttattatcaagcctttatcgattgggaacaggcacgccatggtctgacattgaaacgcgattggattcgatttggtagtggcgtggtcaattcgttgtacgcgctcgttaatattttaacgcaaccaggtgatggcgttttgattctgacgcccgtgtactatccatttttcaacgcggtgcgggataatcatcgtcagctggtcacatctgagttacgcaatgatcgtggtcactatacgattgactttgaagatgtggaacagcaaattaaggcgaacgaggtgcggttgttcattcaatgttcgccacataatcctgtggggcggatctggtctgaagatgaagagacacggctgttagcattatgtgaacagtatcatgtgcttgttatttcagatgaaattcaccaggatttagaaattgatcggaccaaccatccgttcaaatcggcgttaacggtagccaatgggcggtttagtaaccgggtgattgtcgtcaatgcaccgtctaagacgtttaatctggcggcgcttttgaactcacatctagtaattcctaatccaaaattacgacaacgctatgatgcacaaatcgggcgcttcagccagactgagatcagcgtcttaggccaggttgccggccaaacggcttatgaatcgggcgcggcctggttagaccagattttggcaatcgtccgtgataattatcattatttatgcaccacctttgccgcccacgcgcctaagttggttttagccgatttgcaggggacgtatttgacgtggctagatttacgcgcgtacgttcagccagctgcaacgaaagcgtttattcaagatcaatgtcagctcgcagttgactttggtgagtggtttagtcctgatgatcaggggtttgttcgcttgaacttagcgactaatccgcaaaatgtgcgtgaggcggctcagcggatcgtcacttgtttatcacaaatgcaaaaataa

SEQ ID NO: 100 - protein, 381 aa, Lactobacillus plantarum
  misc_feature: cystathionine-beta-lyase
MKFETQLIHGGISEDATTGATSVPIYMASTFRQTKIGQNQYEYSRTGNPTRAAVEALIATLEHGSAGFAFASGSAAINTVFSLFSAGDHIIVGNDVYGGTFRLIDAVLKHFGMTFTAVDTRDLAAVEAAITPTTKAIYLETPTNPLLHITDIAAIAKLAQAHDLLSIIDNTFASPYVQKPLDLGVDIVLHSASKYLGGHSDVIGGLVVTKTPALGEKIGYLQNAIGSILAPQESWLLQRGMKTLALRMQAHLNNAAKIFTYLKSHPAVTKIYYPGDPDNPDFSIAKQQMNGFGAMISFELQPGMNPQTFVEHLQVITLAESLGALESLIEIPALMTHGAIPRTIRLQNGIKDELIRLSVGVEASDDLLADLERGFASIQAD

SEQ ID NO: 101 - DNA, 1146 bp, Lactobacillus plantarum
  misc_feature: Coding sequence of SEQ ID NO 100
atgaaatttgaaacccaattaattcacggtggtatcagtgaggatgccactactggcgcgacttcggtacccatctacatggcctcgaccttccgccaaacaaaaatcggtcaaaatcaatacgaatattcacggacgggaaatccaacccgggccgccgtcgaagcattaattgccaccctcgaacatggcagcgctggcttcgcatttgcttctggctccgctgccattaataccgtcttctcactattctcggctggtgatcacattattgtgggaaatgatgtctacggtggcaccttccgcttgatcgacgccgttttgaaacactttggcatgacttttacagccgtagatacgcgtgacttggccgccgttgaagccgcaattacccccacaactaaggcgatttatttggaaacaccgacgaacccgttattacacattacggatattgctgccattgcgaagctcgcgcaagcacacgatttactgagtatcatcgacaacaccttcgcctccccatacgtccagaagcccctggatttaggcgttgacattgttttacacagtgcttccaagtatctcggtggtcacagtgatgttatcggtggcttggttgtcaccaagacgccagcacttggcgaaaaaatcggctacttgcaaaatgccatcggtagtattttggccccgcaagaaagctggctattacaacgtggtatgaagactctggcattgcgcatgcaagcccacctgaataatgccgctaaaatctttacttacttaaagtctcacccagcagttactaagatttactatccaggcgatcctgataatcccgatttttcgattgccaagcaacagatgaatggcttcggcgcaatgatctcgtttgaattacaaccaggaatgaacccccagaccttcgttgaacatttacaagtcatcacgctcgccgaaagtctcggagcattggaaagtttaattgaaattccagccttaatgactcacggtgccatcccacgcacaattcggctacagaatggcatcaaagacgagctgattcgcttatcagtcggtgttgaagccagtgacgatttgttagcagaccttgagcgcgggttcgctagcattcaggcagattaa

SEQ ID NO: 102 - protein, 390 aa, Lactobacillus plantarum
  misc_feature: cystathionine-beta-lyase
MPFDFETVLNRRHTNAVKWDVADDELPMWVADMDFETAPVIKQALIKRAQFGVFGYEEVPMAYYEAVADWWATEHNFRPQIEWLMFCTGVVPAISSIVRKMTAAGDNVLVQAPVYNIFYHSIENNGRHTLSSDLVARDASYAIDWDDLTKKLADPLTTMMILCNPQNPIGVVWSRATLQRLGELCLKYHVLVVADEIHCDLTLNGNAYTPFSSLTAPVAHNSISCISPSKTFNVAALHAATLIIPDAHVRALVSRGINNDELAEPNSFAIPASIAAYTEGHDWVAALRTKLAANQRQVQTYLTAELPQVKLVEAQATYLLWLDVSQVTTDSAALAAFIRQRTGLFLSAGDVYRGDGNRFLRLNVACPTTTLTDGLARLKAGITAYQQVQA

SEQ ID NO: 103 - DNA, 1173 bp, Lactobacillus plantarum
  misc_feature: Coding sequence of SEQ ID NO 102
atgccgtttgattttgagaccgtgcttaatcggcggcacacgaatgccgttaagtgggacgtcgcggatgatgagttgccgatgtgggtcgctgacatggactttgaaacggcaccagttatcaagcaagcgttgataaaacgcgcccagtttggcgtcttcggttacgaagaagtccccatggcttactatgaagccgtggcagactggtgggcaacggaacacaacttccgtccgcaaattgagtggttgatgttctgcaccggcgtcgttccggcgatttcatcaattgttcgtaaaatgacagcggctggcgataacgtgttagttcaggctccggtttataatatcttctatcattcaattgaaaacaatggccggcataccttgagtagtgacttagtagcacgtgacgcgtcgtacgcgattgattgggatgatttgacgaagaaattggctgatccgctaacaacgatgatgattttgtgtaatccgcagaatccaatcggcgtcgtctggtcacgtgcgacgttgcagcgcctcggcgagctgtgcttgaaataccatgtgctagtcgttgccgatgagattcattgtgacttgacccttaacggtaacgcgtacacgccattttcatcattgactgcgccagtagcacataacagtatcagctgtatctcgccaagcaagactttcaacgtggcagccttacacgcggcaacgttgattattcccgatgcccacgtccgcgcattagtgagccgggggatcaacaatgatgaattggctgaacccaattcgtttgcgattccagccagcattgcggcctatactgaaggtcatgactgggtagccgcattacggaccaaactagccgccaatcaacgccaagttcagacttatttgaccgctgaattgccgcaagtcaagttggttgaggcccaagcgacgtatctattatggttggacgtgagtcaagtgacaaccgacagtgcggctttggctgcttttattcgccaacgaaccgggttgtttttatcagcgggcgatgtttaccgtggtgatggcaaccgcttcttgcgactgaatgtggcttgtccaacgactaccctgacggatggattggcacgattaaaagctggtattacggcttaccaacaagttcaagcatag

SEQ ID NO: 104 - protein, 387 aa, Lactobacillus plantarum
  misc_feature: cystathionine-beta-lyase
MQYDFNKVINRRGTYSTQWDYIQDRFGRSDILPFSISDTDFPVPVGVQEALEQRIKHPIYGYTRWNNEDYKNSIINWFSSQNQVTINPDWILYSPSVVFSIATFIRMKSAVGESVAVFTPMYDAFYHVIEDNQRVLAPVRLGSAQQDYSIDWDTLKAVLKQTATKILLLTNPHNPTGKVFSDDELKHIVALCQQYNVFIISDDIHKDIVYQKAAYTPVTEFTTKNVVLCCSATKTFNTPGLIGAYLFEPEAELREMFLCELKQKNALSSASILGIESQMAAYNTGSDYLVQLITYLQNNFDYLSTFLKSQLPEIRFKQPEATYLAWMDVSQLGLTAEKLQDKLVNTGRVGIMSGTTYGDSHYLRMNIACPISKLQEGLKRMEYGIRS

SEQ ID NO: 105 - DNA, 1164 bp, Lactobacillus plantarum
  misc_feature: Coding sequence of SEQ ID NO 104
atgcaatatgattttaataaggttataaatcgtagagggacatacagtactcagtgggattatattcaagatcgctttggtcgttctgacattctaccattttcaatttcagatactgactttccggttcccgttggcgtccaagaggcgcttgaacagcgtattaagcatcctatttatggttatacacgctggaataatgaggattacaaaaatagtattattaattggtttagctctcaaaatcaagttactataaacccagattggattttatatagtcccagtgttgttttttcaattgccacctttattcgaatgaagtcagccgttggagaaagtgtagcggtcttcactcctatgtatgacgccttttatcatgtgattgaggataatcagcgggtgttagcgccggtcagactaggcagtgcacaacaagactatagtatcgattgggatactttgaaagctgttttaaagcaaacagcaacaaaaattttacttttgactaatccacataatcctaccgggaaggtcttttcagatgatgaattgaagcatatagttgcactatgtcaacaatataatgtctttataatttcagatgatattcataaggacattgtgtatcaaaaggcagcatatacgcctgtaaccgaatttacaactaagaatgtggtcctatgttgttcagctactaaaacttttaatacccctgggttgattggcgcatatttatttgagcctgaggctgaactacgtgagatgtttttatgtgaattaaagcaaaaaaatgctttatcatcagctagcatccttggaattgaatctcagatggctgcttataatactggaagtgactatttagtacaactcataacgtatttgcaaaataactttgattatctatctactttcttaaaaagtcagttaccagagattagatttaagcagcctgaagcgacttatttggcttggatggatgtctcgcaattggggctaacggctgaaaaactacaagataaacttgttaatacgggtcgagttgggatcatgtcggggacaacatatggtgacagtcattatttacgtatgaatattgcttgtcctatttctaaattgcaggaaggactgaaaagaatggagtacgggatccgttcgtaa

SEQ ID NO: 106 - protein, 373 aa, Lactobacillus plantarum
  misc_feature: cystathionine-gamma-synthase
MTKQAEKLHIETRLAQAGNRSDDEKTGAISAPIYLSTAYRHAGLGQSTGFDYPREAQPTRCILERVLAEMEHGIAAYALTSGMAAIQLVFTLFNSGDKIIVSDDLYGGSYRFFDLLHDHYHLDFAVWDGQDQATLAALADDQTVALWLETPSNPTMKVIDITATAATAHAHDLKLIVDNTFYTPLIQKPLDLGADIVVHSATKYLAGHNDVLAGAVVVKSQADADALEFNLVTTGAVLDPFDAWLLLRSLKTLPLRLHQQEANAQELVTVLEADEHVERVLYSGRGGMISFYLATGTDVDTFLRALNVISFAESLGGVESLLTVPAVQTHADLTEEQRQSKGITANLLRLSVGIENSADLAADLKQALIRATK

SEQ ID NO: 107 - DNA, 1122 bp, Lactobacillus plantarum
  misc_feature: Coding sequence of SEQ ID NO 106
atgacaaaacaagctgagaaattacacattgaaacgcggttagcacaagctggtaatcggagtgatgatgagaagaccggggcaatttcggcgcccatctacttgtcgacagcatatcgacatgctggtttaggacaatctacgggatttgactacccgcgcgaggcccaaccgacccgctgtattttggaacgagtgctggcagagatggaacacggcatcgctgcgtatgcgctgacttctggcatggcggcaatccagctcgttttcacgcttttcaatagtggtgacaaaattattgtgtctgatgacttatacgggggttcgtaccgcttctttgacctcttacacgaccattatcatttggattttgctgtctgggatgggcaggatcaagcgacattggcagcactggcagacgatcaaaccgttgccctgtggttggaaacgccatcgaatccaaccatgaaggtcatagatattacggccacagcggctaccgcacacgcccatgacctcaagttaatcgttgataatactttctatacaccgttgattcaaaagccactggatctaggcgcagacatcgtcgttcattccgcaactaagtatttagccggtcataatgatgttttagccggtgcagtggtagtcaagtcgcaggcggatgctgatgcgcttgaatttaatctggtgacgactggtgcggtccttgacccgttcgacgcttggctgctcttacggagcttgaagactttgccattacgtttgcatcagcaggaggctaatgcgcaggaactcgttacggtccttgaggcagatgaacacgttgaacgcgtcttatattcaggacgtggtggaatgattagtttttatctcgctacgggtaccgacgtcgatacctttttacgagcattgaacgtaatctcgtttgcggaaagtcttggcggtgtcgagagcctgttaacagttccggcagttcagacacacgcagatttaacggaagagcaacgacaatcaaaggggatcaccgctaacttattgcggctatccgttgggatcgaaaatagtgctgatttagcagcggatctcaagcaagcgctgatccgagcaacaaaataa

SEQ ID NO: 108 - protein, 230 aa, Lactobacillus plantarum
  misc_feature: adenosylhomocysteine nucleosidase
MKFGIICAMEEELKTLRDALQDEKIVNIKDITFYDGTIDQQEVVLVQSGIGKVQAGMTTALMITNFDVDVVINSGSAGGIGAGLHVGDVVVAKATAYHDVDATAFDYEYGQLPQQPLYYETDANWVKQIMAAAKATGLNAKTGLIVSGDQFIAGKVATDKILAHFPEALSSEMEGAAIGQACHQFQTPYVVIRAMSDVGDENAGVSFDEFILEAGKQSAAMLLALFAAQN

SEQ ID NO: 109 - DNA, 693 bp, Lactobacillus plantarum
  misc_feature: Coding sequence of SEQ ID NO 108
atgaaatttggaattatttgtgcgatggaagaagaacttaaaacgttacgggacgctttgcaagacgaaaagatcgttaatattaaggatattacgttctatgatgggacaattgatcagcaggaagttgtcttagtgcaatcaggcattggtaaagtgcaagctggtatgactacggctttaatgattactaactttgatgtggacgttgtcatcaactctggttctgcaggtgggattggcgctggtttgcatgtcggtgacgttgtcgtagctaaggcaactgcttatcacgatgttgatgcgacggcctttgactacgaatatggccaattaccacaacagccactatactacgaaacggatgccaattgggtaaagcaaatcatggcggcggccaaggcaaccggtttgaatgccaagactggactgattgtttccggtgaccaatttatcgcaggtaaggtggctaccgataagattttagcacactttccagaagcactatctagtgaaatggagggcgccgcaattggtcaagcttgtcaccagttccagacgccatatgtggtgattcgcgcgatgtccgatgtcggtgatgaaaatgccggtgttagttttgacgaatttatcctggaagcgggtaagcagtccgcagcgatgttacttgcattatttgcggcgcaaaattag

SEQ ID NO: 110 - protein, 398 aa, Lactobacillus rhamnosus
  misc_feature: cystathionine-beta-lyase
MSQFNRLFDRKAGNARKWADQVLAEKFGLTANAIPMDLADFDFPVAPPIHQAIMTRAAVPDYSYTYIPEAFYETVIAWNARRFGLQLEKDWIKLSYGTVPTLHYLVQAFTEQNEAVLINTPAYDPFAEAVRNNHRQLATSPLMLVDNRYEFDFADMAAKMQRPDVKMFILCSPQNPSGRVWTAEELARVGELCRQHQVLLISDEIHRDIVYPGVTFTSIWQACPAIQSQSILCLSPNKAFNLGGLKTSYVVIPDPAIRARLNAQFVANSITSPNSFAVPALIAAYTDCDAWLDEMVAYVAANFAYLVNRLQVIPEIQVMPSDSGFLAWLDTQRLFPDETAMKRFFTAADLSCVVGSYFVADGGGFVRLNIGMPRPLLKEALDRMVATYATWPRIGQEA

SEQ ID NO: 111 - DNA, 1197 bp, Lactobacillus rhamnosus
  misc_feature: Coding sequence of SEQ ID NO 110
atgagccaatttaatcgcctttttgatcgcaaagccggcaatgcgcggaagtgggcggatcaagtgctcgcggaaaaattcgggctcacggccaatgcgattcccatggacttggccgattttgacttcccggttgccccgccgattcaccaagccattatgacacgcgcggccgtacctgactacagttacacttacatccccgaagccttttatgagacagtcatcgcctggaacgcgcgccgcttcgggttgcaactggaaaaagactggatcaaactcagctatggcaccgtgccgaccctccactacttggttcaggcattcaccgaacaaaatgaagcggtgttaatcaacacgccggcctatgatccgtttgctgaagccgtgcgcaataaccaccgtcagctcgccaccagtccgctcatgcttgtggacaatcgctacgaattcgactttgcagacatggccgctaaaatgcaacggccggacgttaagatgttcattctctgcagtccgcaaaatccgtccggccgcgtctggacagctgaagaactagcacgcgtgggtgaactttgccgccagcatcaggttttactcattagtgacgaaattcaccgggacattgtttatcccggcgtcacatttaccagcatctggcaagcctgcccagctattcaatcccaaagtattctttgtttatcacccaataaagctttcaatttaggcggtctgaaaacgtcctatgtcgtcatccctgacccagcgatccgcgcccgactcaacgctcaattcgtcgccaattcgattacctcacctaatagctttgccgtgccagccctcatcgctgcttatacggattgtgatgcgtggctggatgaaatggtggcttatgtcgccgctaactttgcctacttggtcaaccgtttgcaagtcataccggaaattcaggtgatgccttccgattccggttttctagcctggctggacacccagcggcttttccctgatgaaacggcgatgaaacgtttctttactgctgctgatttgtcctgtgtggtcggcagctattttgtggctgatggtggcggcttcgtgcgtctcaacatcggcatgccgcgcccgttactcaaagaagccttggatcgcatggtcgcaacttatgcaacttggccacgaattggacaagaagcctga

SEQ ID NO: 112 - protein, 378 aa, Lactobacillus rhamnosus
  misc_feature: cystathionine-beta-lyase
MTQFNTKLVHGPQLNVDQAGAIVPPVYQSAMFRFAPDGQETHWDYARSGNPTREYLERQIATLENGDAGFAFSSGVAAIATVLAIFPDHSHFIIGDSLYSGTDRLINQYFSQHGLTFTPVDTRDLAAVEAAIRPETKAIFFETFSNPLLKVSSVKAISALAKTHDLLTIVDNTFLTPYYQRPLDLGADIVLHSATKYLGGHGDLIAGLVVSAHPDLSEKLAFLQNTIGAILSPLDCSLVTRGIATLSVRLDRETANAQAVAEFLAQHPDVAHVYYPGLKTDPGYALAQKETTGASGLLTIKLADNIDPLKFVNSTKIFDFADSLGTISSLVKLPWFKLPEDKRADSGLTPQHVRIAIGLEDQQDLIDDLKQALAAAEK

SEQ ID NO: 113 - DNA, 1137 bp, Lactobacillus rhamnosus
  misc_feature: Coding sequence of SEQ ID NO 112
atgacccaattcaataccaaactcgttcatggaccacaactaaatgtcgaccaagccggtgctatcgtgccacctgtataccaaagtgccatgttccgctttgctcctgatggtcaggaaacccactgggactatgcgcgcagtggtaacccgacccgcgaatacttggaacgtcagattgctacgctagaaaatggtgatgccggctttgcgttttccagcggcgttgcagcgattgcaacggtgctcgcgatttttcccgaccacagtcacttcattattggtgattcgctctacagtggcaccgatcgcctcattaaccagtatttttctcaacacggcctgacctttacaccggtggatacgcgtgatctggcagcggtggaagccgccatccgccccgaaactaaagcaattttctttgagactttttccaatccgctcctcaaagtcagcagcgtcaaggccatcagtgccctcgccaaaacccatgatctgctaacgattgtcgacaacacgttcttaaccccttattaccagcggccacttgacctcggtgccgacattgttctacacagcgccaccaaatacctcggcggccacggtgacctcattgccggcctcgttgtctccgctcaccccgacctcagcgagaagctcgctttcctgcaaaacacgatcggtgccattttaagcccgcttgactgtagcctcgtcacccgcggcattgccaccctctccgttcgccttgatcgtgaaactgcaaacgcccaagccgtcgccgaatttctagcgcagcacccagacgttgcccacgtttactaccccggacttaaaaccgatcccggttacgcattagcccaaaaagaaaccacgggtgccagcggactcttaacgatcaaactagccgacaacattgatcccttaaagttcgttaacagcaccaaaattttcgactttgccgactcacttggtaccatctccagtctggtcaaactaccttggtttaagctcccggaagacaaacgcgccgattctggtttgacaccgcaacatgtccggattgcaattggcttggaggatcagcaggacttgattgacgatctaaagcaggcactggcagcagcggaaaaatag

SEQ ID NO: 114 - protein, 391 aa, Lactobacillus rhamnosus
  misc_feature: cystathionine-beta-lyase
MDTHLVDRHNSNAVKWDGLKEEFGRADLLPMWIADTEFKAPQAVLDALTARVKEGTFGYSIRPQSYYEAFINWQKERHGITVEPEWMRFGVGVVKSLYAMVNWLTEPGDPVLIMQPVYYPFMNAINDLGRKVVSVDLQLTADGWRMDFDQLEKTLAANEIKAMILCSPHNPVGRIWTRDELEQLFAITSRYDVTVVSDEIHGDLEVSGPKFTSALQVVEGKARKKLVVLNAPSKTFNLAALLNSHIIIPDQALRTSYDAFIKQLHPVDTSLMGQVAGEAAYRHGAAWLDQVLQVVRYNYRQLQAGLAAAAPQATLADLQGTYLAYVDIGAYVAPSQIKDFVEGVCGLAVDYGAWFSPQTATYIRLNLATDPKLVAEAINRLTTHLAQQPQR

SEQ ID NO: 115 - DNA, 1176 bp, Lactobacillus rhamnosus
  misc_feature: Coding sequence of SEQ ID NO 114
gtggacacgcacttagttgatcgtcataatagcaatgctgtgaagtgggacggtctgaaagaagaatttggccgggctgacttgttgcctatgtggattgccgacactgagtttaaggcgcctcaagcagttttggatgcattgacagctcgcgtcaaggaagggacgtttggctattccattcgcccgcagtcttattacgaagccttcattaactggcaaaaggaacgacatggcattacggttgaacctgagtggatgcgttttggcgttggcgttgtcaaatcactgtatgcgatggtgaactggctgacagaacctggtgatccggtcctcatcatgcagccggtttattatccctttatgaatgccattaatgatcttggacgtaaagtcgtatcagttgacttgcaattaaccgctgatggttggcgcatggattttgaccaattagaaaagaccttggcggcgaatgaaattaaagcgatgattctgtgttcaccgcacaatccggttggtcggatctggacccgagatgagttagaacaactttttgccatcacaagtcggtatgatgtgacagtggtttctgatgaaattcacggtgatcttgaagtgagtgggccgaagtttacatccgctctacaggtcgttgaaggtaaagctcgaaaaaagcttgttgtgctcaatgcgccgtcaaaaacatttaatttagccgccttgctgaattcacacattattattcccgatcaagcgttgcgtacgagttatgatgccttcattaagcagctgcatccggttgatacgagcttgatggggcaagtggccggtgaagctgcttatcggcatggtgctgcttggttagatcaggtcttacaagtggttcgctacaattatcggcaactgcaagctggtttagccgcggcggccccacaagcgaccctggccgatttacaagggacttatttggcttatgttgatattggtgcttatgttgcgccaagtcagatcaaagactttgttgaaggggtgtgcggattggccgttgattatggtgcatggttttcacctcaaacggcaacttatattcgtttaaatttagctactgatcctaagcttgttgccgaggcgattaaccgattaaccactcatttggcacagcagccgcagcggtga

SEQ ID NO: 116 - protein, 236 aa, Lactobacillus rhamnosus
  misc_feature: adenosylhomocysteine nucleosidase
MIKVGVICAMEEEIRTLLAKQTHQQETVIASQHYFEGQIDGVDVILVQSGIGKVQAAMTTALLLATFKPDVVVNTGSAGGIGRGLAIGDVVISSGVAYHDADATAFGYLPGQLPQQPQIFEAGMSYVRQIQAAATATGLTSKVGLIVSGDQFINGKEAIARILKIYPQALASEMEGAAVGQVAKEFHTPFVVIRAMSDVADEQSGVDFDKFVIKAGEQSAAMLLQFFSHLQRSQHN

SEQ ID NO: 117 - DNA, 711 bp, Lactobacillus rhamnosus
  misc_feature: Coding sequence of SEQ ID NO 116
ttgattaaggtcggggttatttgtgcgatggaagaagaaattcggacgttactggctaaacagactcatcagcaagaaacggtgattgccagccagcattactttgaaggccaaatagatggtgttgatgttattttggtacaatccggaattggcaaggtacaagcagcgatgacgactgccttgctgttagcgacttttaagccggatgttgttgtcaatactggcagtgccggtggtatcggtcgcggtttggcgatcggtgatgttgtgatttcaagcggtgtggcttatcatgatgcggatgcgaccgcgtttggctatttgccggggcagttgccacaacagccgcagatttttgaagccggcatgagttatgtgcgccagattcaagctgctgcgacggcgaccggcttgacgtcaaaagtcggcttgattgtttcgggcgatcagtttattaatggcaaagaggcgattgcccgaattttgaaaatttatccacaagcgcttgccagtgagatggaaggcgcagcggttggccaggttgccaaggaatttcacaccccgtttgttgttattcgggcaatgagcgatgtggctgatgagcagtctggggttgatttcgacaaatttgtgatcaaagcaggggagcaaagtgctgcgatgttgttacagttctttagtcatttgcaacgatcacagcataattag

(19) United States
(12) Patent Application Publication
(10) Pub. No.: US 2018/0280452 A1
(43) Pub. Date: Oct. 4, 2018

(54) USE OF BACTERIAL METHIONINE METABOLISM FOR LIFESPAN EXTENSION

(71) Applicant: Brigham Young University, Provo, UT (US)

(72) Inventors: John M. Chaston, Provo, UT (US); Melinda L. Koyle, Provo, UT (US)

(21) Appl. No.: 15/943,481

(22) Filed: Apr. 2, 2018

Related U.S. Application Data
(60) Provisional application No. 62/479,659, filed on Mar. 31, 2017.

(51) Int. Cl. A61K 35/741; C12N 15/52; C12P 19/40; A61K 31/205

(52) U.S. Cl. CPC A61K 35/741; A61K 31/205; C12P 19/40; C12N 15/52

(57) ABSTRACT

The disclosure generally relates to microorganisms, methods, and compositions for reducing methionine content of a diet of a subject or extending a lifespan of the subject and processes for preparing the microorganisms and compositions.

### CROSS-REFERENCE TO RELATED APPLICATIONS

This application claims the benefit of U.S. Provisional Application No. 62/479,659 filed Mar. 31, 2017, the disclosure of which is incorporated in its entirety by reference herein.

### SEQUENCE LISTING

The text file Sequences_001_ST25.txt of size 301 KB created Apr. 2, 2018, filed herewith, is incorporated in its entirety by reference herein.

### TECHNICAL FIELD

The disclosure generally relates to microorganisms, methods, and compositions for reducing methionine content in a diet of a subject or extending a lifespan of the subject and processes for preparing the microorganisms and compositions.

### BACKGROUND

The question of how animals age, and how the aging process can be slowed, is of paramount interest. The use of model organisms, especially Caenorhabditis elegans and Drosophila melanogaster have helped us to better understand mammalian and human aging. For example, such models have helped reveal that dietary restriction, reduction in oxidative stress, and reductions in inflammation can all promote a long and healthy lifestyle (MCISAAC, '16). In particular, dietary restriction extends longevity across the tree of life, underscoring its potential as a therapeutic target. In some cases, restriction of individual nutrients through nutrient allocation or acquisition can fully or partially reproduce the longevity-promoting benefits of total calorie restriction.

### SUMMARY

The disclosure generally relates to microorganisms, methods, and compositions for reducing methionine content of a diet of a subject or extending a lifespan of the subject and processes for preparing the microorganisms and compositions. The disclosure also relates to populating an intestinal microbiome with probiotic microorganisms modified to have enhanced rates of methionine to cysteine conversions or reduced rates of methionine recycling relative to the modified organisms devoid of the modifications.

In various embodiments are disclosed probiotic bacteria including a heterologous polynucleotide encoding a cystathionine-β-synthase or a cystathionine-γ-lyase and operably linked to a promoter polynucleotide, wherein the probiotic bacterium is derived from a bacteria strain incapable of reverse transsulfurylation.

In various embodiments are disclosed probiotic bacteria including a constitutive promoter polynucleotide operably linked to a polynucleotide encoding a cystathionine-γ-synthase, a cystathionine-β-lyase, or a adenosylhomocysteine nucleosidase.

In various embodiments are disclosed probiotic bacteria including a mutation in a homologous polynucleotide that encodes a protein selected from at least one of a S-ribosylhomocysteine lyase, methionine synthase, homoserine O-acetyltransferase, 5-methyltetrahydropteroyltriglutamate-homocysteine methyltransferase, or a cobalamin synthase W domain-containing protein 1 or in a homologous promoter polynucleotide operably linked to the homologous polynucleotide, wherein the mutation reduces activity or expression of the protein.

In various embodiments are disclosed methods for reducing methionine content of a diet of a subject or enhancing the lifespan of the subject including administering a composition having an amount of probiotic bacteria in a range from about 10³ to about 10¹⁵ colony forming units (cfu) per gram of the composition to a subject, wherein a bacterium of the probiotic bacteria, colonizing an intestinal microbiome of the subject, metabolizes methionine at a rate greater than a rate the bacterium synthesizes or recycles methionine when the subject digests a methionine containing substance.

### BRIEF DESCRIPTION OF THE DRAWINGS

For a further understanding of the nature, objects, and advantages of the present invention, reference should be had to the following detailed description, read in conjunction with the following drawings, wherein like reference numerals denote like elements and wherein

FIG. 1 shows the bacterial effects on the mean lifespan of female Drosophila melanogaster.

FIG. 2 shows the bacterial effects on the mean lifespan of male Drosophila melanogaster.

FIG. 3 shows a diagram of the methionine cycle, including the methionine cycle, one carbon metabolism, transsulfuration, and vitamin B6-dependent interconversion of glycine and serine.

FIG. 4 shows average lifespans of female Drosophila melanogaster monoassociated with Escherichia coli mutant strains.

FIG. 5 shows average lifespans of male Drosophila melanogaster monoassociated with Escherichia coli mutant strains.

FIGS. 6, 7, 8, and 9 show differences in metabolite levels in flies reared with wild-type bacteria or flies reared with pdxB or pdxK mutants.

FIG. 10 shows mean lifespan differences in flies reared with wild-type bacteria or flies reared with pdxB, pdxK, or glyA mutants.

FIG. 11 shows mean lifespan differences in flies reared with wild-type bacteria or flies reared with metE or luxS mutants and living at least 48 days.

FIG. 12 shows effects on the methionine content of diets administered to flies reared with wild-type bacteria or flies reared with metE or luxS mutants.

FIG. 13 shows an metobolomic analysis of diets showing differences in S-ribosylhomocysteine abundance of flies reared with pdxB, pdxK, or luxS mutants as compared to their wild-type counterparts.

FIG. 14 shows mean lifespan differences of flies reared bearing CBS::CGL-expressing bacteria as compared to their wild-type counterparts.

FIG. 15 shows effects on the methionine content of diets administered to flies reared with CBS: :CGL-expressing bacteria as compared to their wild-type counterparts.

FIGS. 16A, 16B, 17A, 17B, 18A, and 18B show models of the effect of bacterial methionine metabolism on the lifespan of Drosophila melanogaster.

### BRIEF DESCRIPTION OF THE SEQUENCES

SEQ ID NO: 1 sets forth an amino acid sequence of a cystathionine-β-synthase from Klebsiella variicola.

SEQ ID NO: 2 sets forth a polynucleotide sequence encoding SEQ ID NO: 1.

SEQ ID NO: 3 sets forth an amino acid sequence of a cystathionine-γ-lyase from Klebsiella variicola.

SEQ ID NO: 4 sets forth a polynucleotide sequence encoding SEQ ID NO: 3.

SEQ ID NO: 5 sets forth an amino acid sequence of a cystathionine-γ-synthase from Klebsiella variicola.

SEQ ID NO: 6 sets forth a polynucleotide sequence encoding SEQ ID NO: 5.

SEQ ID NO: 7 sets forth an amino acid sequence of a cystathionine-β-lyase from Klebsiella variicola.

SEQ ID NO: 8 sets forth a polynucleotide sequence encoding SEQ ID NO: 7.

SEQ ID NO: 9 sets forth an amino acid sequence of an adenosylhomocysteine nucleosidase from Klebsiella variicola.

SEQ ID NO: 10 sets forth a polynucleotide sequence encoding SEQ ID NO: 9.

SEQ ID NO: 11 sets forth a polynucleotide sequence of an operon including SEQ

ID NO: 2 (nt 1 to nt 1371) and SEQ ID NO: 4 (nt 1393 to nt 2531).

SEQ ID NO: 12 and SEQ ID NO: 13 are forward and reverse primers with restriction enzyme recognition sequences for cloning SEQ ID NO: 11.

SEQ ID NO: 14 is a sequencing primer that is upstream from the operon (SEQ ID NO: 11) from Klebsiella variicola.

SEQ ID NO: 15, SEQ ID NO: 16, SEQ ID NO: 17, SEQ ID NO: 18, and SEQ ID NO: 19 are sequencing primers specific to the operon (SEQ ID NO: 11) from Klebsiella variicola.

SEQ ID NO: 20 sets forth an amino acid sequence of a cystathionine-β-synthase like from Homo sapiens.

SEQ ID NO: 21 sets forth a cDNA polynucleotide sequence encoding SEQ ID NO: 20.

SEQ ID NO: 22 sets forth an amino acid sequence of a cystathionine-β-synthase from Homo sapiens.

SEQ ID NO: 23 sets forth a cDNA polynucleotide sequence encoding SEQ ID NO: 22.

SEQ ID NO: 24 sets forth an amino acid sequence of a cystathionine-γ-lyase from Homo sapiens.

SEQ ID NO: 25 sets forth a cDNA polynucleotide sequence encoding SEQ ID NO: 24.

SEQ ID NO: 26 sets forth an amino acid sequence of a cystathionine-β-synthase from Mus musculus.

SEQ ID NO: 27 sets forth a cDNA polynucleotide sequence encoding SEQ ID NO: 26.

SEQ ID NO: 28 sets forth an amino acid sequence of a cystathionine-γ-lyase from Mus musculus.

SEQ ID NO: 29 sets forth a cDNA polynucleotide sequence encoding SEQ ID NO: 28.

SEQ ID NO: 30 sets forth an amino acid sequence of a cystathionine-β-synthase from Pseudomonas aeruginosa (PAO1).

SEQ ID NO: 31 sets forth a polynucleotide sequence encoding SEQ ID NO: 30.

SEQ ID NO: 32 sets forth an amino acid sequence of a cystathionine-γ-lyase from Pseudomonas aeruginosa (PAO1).

SEQ ID NO: 33 sets forth a polynucleotide sequence encoding SEQ ID NO: 32.

SEQ ID NO: 34 sets forth an amino acid sequence of a cystathionine-β-synthase from Streptomyces venezuelae.

SEQ ID NO: 35 sets forth a polynucleotide sequence encoding SEQ ID NO: 34.

SEQ ID NO: 36 sets forth an amino acid sequence of a cystathionine-β-synthase from Streptomyces venezuelae.

SEQ ID NO: 37 sets forth a polynucleotide sequence encoding SEQ ID NO: 36.

SEQ ID NO: 38 sets forth an amino acid sequence of a cystathionine-γ-lyase from Streptomyces venezuelae.

SEQ ID NO: 39 sets forth a polynucleotide sequence encoding SEQ ID NO: 38.

SEQ ID NO: 40 sets forth an amino acid sequence of a cystathionine-γ-lyase from Streptomyces venezuelae.

SEQ ID NO: 41 sets forth a polynucleotide sequence encoding SEQ ID NO: 40.

SEQ ID NO: 42 sets forth an amino acid sequence of a cystathionine-β-lyase from Streptomyces venezuelae.

SEQ ID NO: 43 sets forth a polynucleotide sequence encoding SEQ ID NO: 42.

SEQ ID NO: 44 sets forth an amino acid sequence of a cystathionine-γ-synthase from Streptomyces venezuelae.

SEQ ID NO: 45 sets forth a polynucleotide sequence encoding SEQ ID NO: 44.

SEQ ID NO: 46 sets forth an amino acid sequence of an adenosylhomocysteine nucleosidase from Streptomyces venezuelae.

SEQ ID NO: 47 sets forth a polynucleotide sequence encoding SEQ ID NO: 46.

SEQ ID NO: 48 sets forth an amino acid sequence of a cystathionine-β-lyase from Helicobacter pylori.

SEQ ID NO: 49 sets forth a polynucleotide sequence encoding SEQ ID NO: 48.

SEQ ID NO: 50 sets forth an amino acid sequence of a cystathionine-β-synthase from Aspergillus nidulans.

SEQ ID NO: 51 sets forth a cDNA polynucleotide sequence encoding SEQ ID NO: 50.

SEQ ID NO: 52 sets forth an amino acid sequence of a cystathionine-γ-lyase from Aspergillus nidulans.

SEQ ID NO: 53 sets forth a cDNA polynucleotide sequence encoding SEQ ID NO: 52.

SEQ ID NO: 54 sets forth an amino acid sequence of a cystathionine-β-lyase from Aspergillus nidulans.

SEQ ID NO: 55 sets forth a cDNA polynucleotide sequence encoding SEQ ID NO: 54.

SEQ ID NO: 56 sets forth an amino acid sequence of a cystathionine-γ-synthase from Aspergillus nidulans.

SEQ ID NO: 57 sets forth a cDNA polynucleotide sequence encoding SEQ ID NO: 56.

SEQ ID NO: 58 sets forth an amino acid sequence of a cystathionine-β-synthase from Leishmania major.

SEQ ID NO: 59 sets forth a polynucleotide sequence encoding SEQ ID NO: 58.

SEQ ID NO: 60 sets forth an amino acid sequence of a cystathionine-β-lyase from Leishmania major.

SEQ ID NO: 61 sets forth a polynucleotide sequence encoding SEQ ID NO: 60.

SEQ ID NO: 62 sets forth an amino acid sequence of a cystathionine-β-lyase from Leishmania major.

SEQ ID NO: 63 sets forth a polynucleotide sequence encoding SEQ ID NO: 62.

SEQ ID NO: 64 sets forth an amino acid sequence of a cystathionine-β-lyase from Oryza sativa.

SEQ ID NO: 65 sets forth a cDNA polynucleotide sequence encoding SEQ ID NO: 64.

SEQ ID NO: 66 sets forth an amino acid sequence of a cystathionine-β-lyase from Oryza sativa.

SEQ ID NO: 67 sets forth a cDNA polynucleotide sequence encoding SEQ ID NO: 66.

SEQ ID NO: 68 sets forth an amino acid sequence of a cystathionine-γ-synthase from Oryza sativa.

SEQ ID NO: 69 sets forth a cDNA polynucleotide sequence encoding SEQ ID NO: 68.

SEQ ID NO: 70 sets forth an amino acid sequence of a cystathionine-γ-synthase from Oryza sativa.

SEQ ID NO: 71 sets forth a cDNA polynucleotide sequence encoding SEQ ID NO: 70.

SEQ ID NO: 72 sets forth an amino acid sequence of a cystathionine-γ-synthase from Oryza sativa.

SEQ ID NO: 73 sets forth a cDNA polynucleotide sequence encoding SEQ ID NO: 72.

SEQ ID NO: 74 sets forth an amino acid sequence of a cystathionine-β-synthase from Saccharomyces cerevisiae.

SEQ ID NO: 75 sets forth a polynucleotide sequence encoding SEQ ID NO: 74.

SEQ ID NO: 76 sets forth an amino acid sequence of a cystathionine-γ-lyase from Saccharomyces cerevisiae.

SEQ ID NO: 77 sets forth a polynucleotide sequence encoding SEQ ID NO: 76.

SEQ ID NO: 78 sets forth an amino acid sequence of a cystathionine-β-lyase from Saccharomyces cerevisiae.

SEQ ID NO: 79 sets forth a polynucleotide sequence encoding SEQ ID NO: 78.

SEQ ID NO: 80 sets forth an amino acid sequence of a cystathionine-β-lyase from Saccharomyces cerevisiae.

SEQ ID NO: 81 sets forth a polynucleotide sequence encoding SEQ ID NO: 80.

SEQ ID NO: 82 sets forth an amino acid sequence of a cystathionine-γ-synthase from Saccharomyces cerevisiae.

SEQ ID NO: 83 sets forth a polynucleotide sequence encoding SEQ ID NO: 82.

SEQ ID NO: 84 sets forth an amino acid sequence of a cystathionine-γ-synthase from Saccharomyces cerevisiae.

SEQ ID NO: 85 sets forth a polynucleotide sequence encoding SEQ ID NO: 84.

SEQ ID NO: 86 sets forth an amino acid sequence of a cystathionine-γ-synthase from Saccharomyces cerevisiae.

SEQ ID NO: 87 sets forth a polynucleotide sequence encoding SEQ ID NO: 86.

SEQ ID NO: 88 sets forth an amino acid sequence of a cystathionine-β-lyase from Arabidopsis thaliana.

SEQ ID NO: 89 sets forth a cDNA polynucleotide sequence encoding SEQ ID NO: 88.

SEQ ID NO: 90 sets forth an amino acid sequence of a cystathionine-γ-synthase from Arabidopsis thaliana.

SEQ ID NO: 91 sets forth a cDNA polynucleotide sequence encoding SEQ ID NO: 90.

SEQ ID NO: 92 sets forth an amino acid sequence of a cystathionine-γ-synthase from Arabidopsis thaliana.

SEQ ID NO: 93 sets forth a cDNA polynucleotide sequence encoding SEQ ID NO: 92.

SEQ ID NO: 94 sets forth an amino acid sequence of a cystathionine-β-lyase from Klebsiella variicola.

SEQ ID NO: 95 sets forth a polynucleotide sequence encoding SEQ ID NO: 94.

SEQ ID NO: 96 sets forth an amino acid sequence of an adenosylhomocysteine nucleosidase from Klebsiella variicola.

SEQ ID NO: 97 sets forth a polynucleotide sequence encoding SEQ ID NO: 96.

SEQ ID NO: 98 sets forth an amino acid sequence of a cystathionine-β-lyase from Lactobacillus plantarum.

SEQ ID NO: 99 sets forth a polynucleotide sequence encoding SEQ ID NO: 98.

SEQ ID NO: 100 sets forth an amino acid sequence of a cystathionine-β-lyase from Lactobacillus plantarum.

SEQ ID NO: 101 sets forth a polynucleotide sequence encoding SEQ ID NO: 100.

SEQ ID NO: 102 sets forth an amino acid sequence of a cystathionine-β-lyase from Lactobacillus plantarum.

SEQ ID NO: 103 sets forth a polynucleotide sequence encoding SEQ ID NO: 102.

SEQ ID NO: 104 sets forth an amino acid sequence of a cystathionine-β-lyase from Lactobacillus plantarum.

SEQ ID NO: 105 sets forth a polynucleotide sequence encoding SEQ ID NO: 104.

SEQ ID NO: 106 sets forth an amino acid sequence of a cystathionine-γ-synthase from Lactobacillus plantarum.

SEQ ID NO: 107 sets forth a polynucleotide sequence encoding SEQ ID NO: 106.

SEQ ID NO: 108 sets forth an amino acid sequence of an adenosylhomocysteine nucleosidase from Lactobacillus plantarum.

SEQ ID NO: 109 sets forth a polynucleotide sequence encoding SEQ ID NO: 108.

SEQ ID NO: 110 sets forth an amino acid sequence of a cystathionine-β-lyase from Lactobacillus rhamnosus.

SEQ ID NO: 111 sets forth a polynucleotide sequence encoding SEQ ID NO: 110.

SEQ ID NO: 112 sets forth an amino acid sequence of a cystathionine-β-lyase from Lactobacillus rhamnosus.

SEQ ID NO: 113 sets forth a polynucleotide sequence encoding SEQ ID NO: 112.

SEQ ID NO: 114 sets forth an amino acid sequence of a cystathionine-β-lyase from Lactobacillus rhamnosus.

SEQ ID NO: 115 sets forth a polynucleotide sequence encoding SEQ ID NO: 114.

SEQ ID NO: 116 sets forth an amino acid sequence of an adenosylhomocysteine nucleosidase from Lactobacillus rhamnosus.

SEQ ID NO: 117 sets forth a polynucleotide sequence encoding SEQ ID NO: 116.

### DETAILED DESCRIPTION

As required, detailed embodiments of the present disclosure are disclosed herein; however, it is to be understood that the disclosed embodiments are merely exemplary and may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art.

Except in the examples, or where otherwise expressly indicated, all numerical quantities in this description indicating amounts of material or conditions of reaction and/or use are to be understood as modified by the word “about”. The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which the present disclosure belongs.

It is also to be understood that this disclosure is not limited to the specific embodiments and methods described below, as specific components and/or conditions may, of course, vary. Furthermore, the terminology used herein is used only for describing particular embodiments and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The terms “or” and “and” can be used interchangeably and can be understood to mean “and/or”.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

The term “microbiome” refers to the totality of microbes (bacteria, fungae, protists), their genetic elements (genomes) in a defined environment. The microbiome may be a gut microbiome (i.e. intestinal microbiome).

The term “bacterium”, “bacteria”, and “strain” are used interchangeably and refers to microorganism(s) having its conventional meaning as used in the art, that is, generally, a low taxonomic rank indicating a genetic variant or subtype of a microorganism (within a defined species). Further, it can be also understood that “bacterium” and “bacteria” are genetically modified bacterium or bacteria.

The term “probiotic” is understood to mean probiotic bacteria that impart benefits to a subject when colonizing the microbiome of the subject. For example, a probiotic bacterium can express competitive exclusion effect against pathogenic microorganisms or intensify disease-resistant properties of subjects through suppressive action of metabolites.

The terms “polynucleotide”, “nucleotide”, “nucleotide sequence”, “nucleic acid” and “oligonucleotide” are used interchangeably in this disclosure. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “heterologous” nucleic acid can refer to a nucleic acid that is not normally or naturally found in or produced by a given bacterium, organism, or cell in nature. The term “homologous” nucleic acid can refer to a nucleic acid that is normally found in or produced by a given bacterium, organism, or cell in nature.

The term “recombinant” is understood to mean that a particular nucleic acid (DNA or RNA) or protein is the product of various combinations of cloning, restriction, or ligation steps resulting in a construct having a structural coding or non-coding sequence distinguishable from endogenous nucleic acids found in natural systems.

The terms “amino acid sequence” or “amino acid” refers to a list of abbreviations, letters, characters or words representing amino acid residues. The amino acid abbreviations used herein are conventional one letter codes for the amino acids and are expressed as follows: A, alanine; C, cysteine; D aspartic acid; E, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine.

The terms “peptide” or “protein” as used herein refers to any peptide, oligopeptide, polypeptide, gene product, expression product, or protein. A peptide is comprised of consecutive amino acids. The term “peptide” encompasses naturally occurring or synthetic molecules.

The terms “construct”, “cassette”, “expression cassette”, “plasmid”, “vector”, or “expression vector” is understood to mean a recombinant nucleic acid, generally recombinant DNA, which has been generated for the purpose of the expression or propagation of a nucleotide sequence(s) of interest, or is to be used in the construction of other recombinant nucleotide sequences.

The term “promoter” or “promoter polynucleotide” is understood to mean a regulatory sequence/element or control sequence/element that is capable of binding/recruiting a RNA polymerase and initiating transcription of sequence downstream or in a 3′ direction from the promoter. A promoter can be, for example, constitutively active or always on or inducible in which the promoter is active or inactive in the presence of an external stimulus. Example of promoters include T7 promoters or lactose (lac) promoters.

The term “operably linked” can mean the positioning of components in a relationship which permits them to function in their intended manner. For example, a promoter can be linked to a polynucleotide sequence to induce transcription of the polynucleotide sequence.

The terms “sequence identity” or “identity” refers to a specified percentage of residues in two nucleic acid or amino acid sequences that are identical when aligned for maximum correspondence over a specified comparison window, as measured by sequence comparison algorithms or by visual inspection, wherein the portion of the sequence in the comparison window may include additions or deletions (i.e., gaps) as compared to the reference sequence (which does not comprise additions or deletions) for optimal alignment of the two sequences. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity.

The term “comparison window” refers to a segment of at least about 20 contiguous positions in which a sequence may be compared to a reference sequence of the same number of contiguous positions after the two sequences are aligned optimally. In a refinement, the comparison window is from 15 to 30 contiguous positions in which a sequence may be compared to a reference sequence of the same number of contiguous positions after the two sequences are aligned optimally. In another refinement, the comparison window is usually from about 50 to about 200 contiguous positions in which a sequence may be compared to a reference sequence of the same number of contiguous positions after the two sequences are aligned optimally.

The terms “complementarity” or “complement” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 4, 5, and 6 out of 6 being 66.67%, 83.33%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 40%, 50%, 60%, 62.5%, 70%, 75%, 80%, 85%, 90%, 95%, 97%, 98%, 99%, or 100%, or percentages in between over a region of 4, 5, 6, 7, and 8 nucleotides, or refers to two nucleic acids that hybridize under stringent conditions.

The term “subject(s)” refers to subjects of any mammalian subject(s) of any mammalian species such as, but not limited to, humans, dogs, cats, horses, rodents, any domesticated animal, or any wild animal.

The disclosure generally relates to microorganisms, methods, and compositions for reducing methionine content of a diet of a subject or extending a lifespan of the subject and processes for preparing the microorganisms and compositions. The disclosure also relates to populating an intestinal microbiome with probiotic microorganisms modified to have enhanced rates of methionine to cysteine conversions or reduced rates of methionine recycling relative to the modified organisms devoid of the modifications.

In various embodiments are disclosed probiotic bacteria including a heterologous polynucleotide encoding a cystathionine-β-synthase or a cystathionine-γ-lyase and operably linked to a promoter polynucleotide, wherein the probiotic bacterium is derived from a bacteria strain incapable of reverse transsulfurylation. In reverse transsulfurylation, an 1-allo-cystathionine intermediate is formed when homocysteine donates its sulfhydryl group to activated serine through a cystathionine-β-synthase; cystathionine is cleaved by cystathionine-γ-lyase to yield cysteine, α-keto-butyrate, and ammonia. The probiotic bacterium of various embodiments can further include a mutation in a homologous polynucleotide that encodes a protein selected from at least one of a S-ribosylhomocysteine lyase, methionine synthase, homoserine O-acetyltransferase, 5-methyltetrahydropteroyltriglutamate-homocysteine methyltransferase, and a cobalamin synthase W domain-containing protein 1 or in a homologous promoter polynucleotide operably linked to the homologous polynucleotide, wherein the mutation reduces activity or expression of the protein.

The promoter polynucleotide of various embodiments is a constitutive promoter polynucleotide or an inducible promoter polynucleotide. Transcription of proteins from heterologous polynucleotides are normally regulated and initiated by a promoter polynucleotide. The constitutive promoter polynucleotide of various embodiments is capable of expressing proteins at high concentration. In various embodiments, the transcript level of the constitutive promoter polynucleotide is about or is at least about 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 10.5-fold, 11-fold, 11.5-fold, 12-fold, 12.5-fold, 13-fold, 13.5-fold, 14-fold, 14.5-fold, 15-fold, 15.5-fold, 16-fold, 16.5-fold, 17-fold, 17.5-fold, 18-fold, 18.5-fold, 19-fold, 19.5-fold, 20-fold, 50-fold, 100-fold, 250-fold, 500-fold, 1000-fold, 2000-fold, 2500-fold, 3000-fold, 3500-fold, 4000-fold, 4500-fold, 5000-fold, 5500-fold, 6000-fold, 6500-fold, 7000-fold, 7500-fold, 8000-fold, 8500-fold, 9000-fold, 9500-fold, or 10000-fold higher than a transcript level of a native promoter for an operon encoding cystathionine-β-synthase or cystathionine-γ-lyase operon. In various embodiments, the transcript level of the constitutive promoter polynucleotide is a range between any two levels listed above. In various embodiments, the transcript level of the constitutive promoter polynucleotide is about 10000-fold or higher than a transcript level of a native promoter for an operon encoding cystathionine-β-synthase or cystathionine-γ-lyase operon. Examples of constitutive promoter polynucleotides or inducible promoter polynucleotides include lac, T7, T7Lac, Sp6, AraBAD, trp, Ptac, pL, λpL, λpR, T6, recA, gal, ara, or hut.

The heterologous polynucleotide operably linked to a promoter polynucleotide of various embodiments can be within a cassette of an expression vector introduced into the probiotic bacteria or stably incorporated within the probiotic bacteria or into a genome of the probiotic bacteria.

In various embodiments, the cystathionine-β-synthase has an amino acid sequence that is or that is at least 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% identical to any one of SEQ ID NO: 1, SEQ ID NO: 20, SEQ ID NO: 22, SEQ ID NO: 26, SEQ ID NO: 30, SEQ ID NO: 34, SEQ ID NO: 36, SEQ ID NO: 50, SEQ ID NO: 58, or SEQ ID NO: 74. In various embodiments, the percent identity is a range between any two percentages listed above. In various embodiments, cystathionine-β-synthase includes cystathionine-β-synthase like.

In various embodiments, the heterologous polynucleotide encoding cystathionine-β-synthase has a nucleotide sequence that is about or that is at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% identical to any one of SEQ ID NO: 2, SEQ ID NO: 11, SEQ ID NO: 21, SEQ ID NO: 23, SEQ ID NO: 27, SEQ ID NO: 31, SEQ ID NO: 35, SEQ ID NO: 37, SEQ ID NO: 51, SEQ ID NO: 59, or SEQ ID NO: 75. In various embodiments, the percent identity is a range between any two percentages listed above.

In various embodiments, the cystathionine-y-lyase has an amino acid sequence that is about or that is at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% identical to any one of SEQ ID NO: 3, SEQ ID NO: 24, SEQ ID NO: 28, SEQ ID NO: 32, SEQ ID NO: 38, SEQ ID NO: 40, SEQ ID NO: 52, or SEQ ID NO: 76. In various embodiments, the percent identity is a range between any two percentages listed above.

In various embodiments, the heterologous polynucleotide encoding cystathionine-γ-lyase has a nucleotide sequence that is about or that is at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% identical to any one of SEQ ID NO: 4, SEQ ID NO: 11, SEQ ID NO: 25, SEQ ID NO: 29, SEQ ID NO: 33, SEQ ID NO: 39, SEQ ID NO: 41, SEQ ID NO: 53, or SEQ ID NO: 77. In various embodiments, the percent identity is a range between any two percentages listed above.

In various embodiments are disclosed probiotic bacteria including a constitutive promoter polynucleotide operably linked to a polynucleotide encoding a cystathionine-γ-synthase, a cystathionine-β-lyase, or a adenosylhomocysteine nucleosidase. The probiotic bacteria of various embodiments can further include a heterologous polynucleotide encoding a cystathionine-γ-synthase or a cystathionine-γ-lyase operably linked to the constitutive promoter polynucleotide or a mutation in a homologous polynucleotide that encodes a protein selected from at least one of a S-ribosylhomocysteine lyase, methionine synthase, homoserine O-acetyltransferase, 5-methyltetrahydropteroyltriglutamate-homocysteine methyltransferase, and a cobalamin synthase W domain-containing protein 1 or in a homologous promoter polynucleotide operably linked to the homologous polynucleotide, wherein the mutation reduces activity or expression of the protein.

In various embodiments, the transcript level of the constitutive promoter polynucleotide is about or is at least about 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 10.5-fold, 11-fold, 11.5-fold, 12-fold, 12.5-fold, 13-fold, 13.5-fold, 14-fold, 14.5-fold, 15-fold, 15.5-fold, 16-fold, 16.5-fold, 17-fold, 17.5-fold, 18-fold, 18.5-fold, 19-fold, 19.5-fold, 20-fold, 50-fold, 100-fold, 250-fold, 500-fold, 1000-fold, 2000-fold, 2500-fold, 3000-fold, 3500-fold, 4000-fold, 4500-fold, 5000-fold, 5500-fold, 6000-fold, 6500-fold, 7000-fold, 7500-fold, 8000-fold, 8500-fold, 9000-fold, 9500-fold, or 10000-fold higher than a transcript level of a native promoter for an operon encoding cystathionine-β-synthase or cystathionine-γ-lyase. In various embodiments, the transcript level of the constitutive promoter polynucleotide is about 10000-fold or higher than a transcript level of a native promoter for an operon encoding cystathionine-β-synthase or cystathionine-γ-lyase operon. In various embodiments, the transcript level of the constitutive promoter polynucleotide is a range between any two levels listed above. Examples constitutive promoter polynucleotides or inducible promoter polynucleotides include lac, T7, T7Lac, Sp6, AraBAD, trp, Ptac, pL, λpL, λpR, T6, recA, gal, ara, or hut.

In various embodiments, the constitutive promoter is a native promoter driving expression of homologous polynucleotides encoding cystathionine-γ-synthase, cystathionine-β-lyase, or adenosylhomocysteine nucleosidase, where the native promoter has been modified to enhance expression levels of cystathionine-γ-synthase, cystathionine-β-lyase, or adenosylhomocysteine nucleosidase. Examples of such modifications can include removal of regulatory sequences such that a base promoter remains or introducing a constitutive promoter at a position within the genome of the bacterium to drive expression of cystathionine-γ-synthase, cystathionine-β-lyase, or adenosylhomocysteine nucleosidase.

In various embodiments, the polynucleotide encoding cystathionine-γ-synthase, cystathionine-β-lyase, or adenosylhomocysteine nucleosidase is a heterologous polynucleotide. The heterologous polynucleotide of various embodiments can be within a cassette of an expression vector introduced into the probiotic bacteria or stably incorporated within the probiotic bacteria or into a genome of the probiotic bacteria.

In various embodiments, the heterologous polynucleotide encoding cystathionine-γ-synthase has a nucleotide sequence that is about or that is least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% identical to any one of SEQ ID NO: 6, SEQ ID NO: 45, SEQ ID NO: 57, SEQ ID NO: 69, SEQ ID NO: 71, SEQ ID NO: 73, SEQ ID NO: 83, SEQ ID NO: 85, SEQ ID NO: 87, SEQ ID NO: 91, SEQ ID NO: 93, or SEQ ID NO: 107. In various embodiments, the percent identity is a range between any two percentages listed above.

In various embodiments, the cystathionine-γ-synthase has an amino acid sequence that is about or that is at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% identical to any one of SEQ ID NO: 5, SEQ ID NO: 44, SEQ ID NO: 56, SEQ ID NO: 68, SEQ ID NO: 70, SEQ ID NO: 72, SEQ ID NO: 82, SEQ ID NO: 84, SEQ ID NO: 86, SEQ ID NO: 90, SEQ ID NO: 92, or SEQ ID NO: 106. In various embodiments, the percent identity is a range between any two percentages listed above.

In various embodiments, the heterologous polynucleotide encoding cystathionine-β-lyase has a nucleotide sequence that is about or that is at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% identical to any one of SEQ ID NO: 8, SEQ ID NO: 43, SEQ ID NO: 49, SEQ ID NO: 55, SEQ ID NO: 61, SEQ ID NO: 63, SEQ ID NO: 65, SEQ ID NO: 67, SEQ ID NO: 79, SEQ ID NO: 81, SEQ ID NO: 89, SEQ ID NO: 95, SEQ ID NO: 99, SEQ ID NO: 101, SEQ ID NO: 103, SEQ ID NO: 105, SEQ ID NO: 111, SEQ ID NO: 113, or SEQ ID NO: 115. In various embodiments, the percent identity is a range between any two percentages listed above.

In various embodiments, the cystathionine-β-lyase has an amino acid sequence that is or that is about at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% identical to any one of SEQ ID NO: 7, SEQ ID NO: 42, SEQ ID NO: 48, SEQ ID NO: 54, SEQ ID NO: 60, SEQ ID NO: 62, SEQ ID NO: 64, SEQ ID NO: 66, SEQ ID NO: 78, SEQ ID NO: 80, SEQ ID NO: 88, SEQ ID NO: 94, SEQ ID NO: 98, SEQ ID NO: 100, SEQ ID NO: 102, SEQ ID NO: 104, SEQ ID NO: 110, SEQ ID NO: 112, or SEQ ID NO: 114. In various embodiments, the percent identity is a range between any two percentages listed above.

In various embodiments, the heterologous polynucleotide encoding adenosylhomocysteine nucleosidase has a nucleotide sequence that is about or that is at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% identical to any one of SEQ ID NO: 10, SEQ ID NO: 47, SEQ ID NO: 97, SEQ ID NO: 109, or SEQ ID NO: 117. In various embodiments, the percent identity is a range between any two percentages listed above.

In various embodiments, the adenosylhomocysteine nucleosidase has an amino acid sequence that is or that is about at least about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, or 100% identical to any one of SEQ ID NO: 9, SEQ ID NO: 46, SEQ ID NO: 96, SEQ ID NO: 108, or SEQ ID NO: 116. In various embodiments, the percent identity is a range between any two percentages listed above.

In various embodiments, the heterologous polynucleotide encoding cystathionine-γ-synthase, cystathionine-β-lyase, or adenosylhomocysteine nucleosidase is operably linked to the constitutive promoter polynucleotide of a second promoter polynucleotide. The second promoter nucleotide of various embodiments can include a constitutive promoter nucleotide polynucleotide or an inducible promoter polynucleotide.

In various embodiments are disclosed probiotic bacteria including a mutation in a homologous polynucleotide that encodes a protein selected from at least one of a S-ribosylhomocysteine lyase, methionine synthase, homoserine O-acetyltransferase, 5-methyltetrahydropteroyltriglutamate-homocysteine methyltransferase, and a cobalamin synthase W domain-containing protein 1 or in a homologous promoter polynucleotide operably linked to the homologous polynucleotide, wherein the mutation reduces activity or expression of the protein.

The probiotic bacterium or the bacteria strain incapable of reverse transsulfurylation of any embodiment can belong(s) to a genus Lactobacillus, Bifidobacterium, Escherichia, Enterococcus, Bacillus, Propionibacterium, Streptococcus, Lactococcus, Pediococcus, or Saccharomyces or belong(s) to an order Lactobacillales.

In various embodiments are disclosed compositions for reducing methionine content of a diet of a subject or extending a lifespan of the subject including a probiotic bacteria of any embodiment and a pharmaceutically acceptable excipient. In various embodiments, the composition includes a plurality of different probiotic bacteria of any embodiment, wherein each of the different probiotic bacteria belongs to a different strain, species, or genus. The plurality of different probiotic bacteria can include or can include at least 2, 3, 4, 5, 6, 7, 8, 9, 10, 11, 12, 13, 14, 15, 16, 17, 18, 19, or 20 different probiotic bacteria. In various embodiments, the number of different probiotic bacteria in the composition is a range between any two numbers listed above. In various embodiments, the plurality of different probiotic bacteria can include 20 or more different bacteria.

In various embodiments, the amount of the probiotic bacteria in the composition is about or is at least about 10³, 10⁴, 10⁵, 10⁶, 10⁷, 10⁸, 10⁹, 10¹⁰, 10¹¹, 10¹², 10¹³, 10¹⁴, or 10¹⁵ colony forming units (cfu) per gram of the composition. In various embodiments, the amount of the probiotic bacteria in the composition is a range between any two cfu per gram listed above. In various embodiments, the amount of the probiotic bacteria in the composition is about 10¹⁵ cfu or more per gram of the composition.

In various embodiments, the pharmaceutically acceptable excipient is a carrier suitable for oral consumption. Examples of carriers include silicon dioxide (silica, silica gel), carbohydrates or carbohydrate polymers (polysaccharides), cyclodextrins, starches, degraded starches (starch hydrolysates), chemically or physically modified starches, modified celluloses, gum arabic, ghatti gum, tragacanth, karaya, carrageenan, guar gum, locust bean gum, alginates, pectin, inulin or xanthan gum, or hydrolysates of maltodextrins and dextrins. In various embodiments, the bacteria is dispersed throughout the carrier.

In various embodiments, the pharmaceutically acceptable excipient is a carrier suitable for oral consumption and the probiotic bacteria is dispersed throughout the carrier. The pharmaceutically acceptable excipient of various embodiments is capable of delivering at least a portion of the amount of the probiotic bacteria to the intestinal microbiome of the subject in an active state.

In various embodiments, the pharmaceutically acceptable excipient includes an extended release phase capable of releasing at least a portion of the amount of the probiotic bacteria to the intestinal microbiome of the subject over a period of time. In other embodiments, the pharmaceutically acceptable excipient includes an immediate release phase capable of substantially immediately releasing at least a portion of the amount of the probiotic bacteria to the intestinal microbiome of the subject.

In various embodiments, the composition can include other prebiotic compounds or probiotic strains. Examples of such probiotic strains include Lactobacillus such as L. plantarum, L. paracasei, L. acidophilus , L. casei, L. rhamnosus, L. crispatus, L. gasseri , L. reuteri, L. bulgaricus; Bifidobacterium such as B. longum, B. catenulatum , B. breve, B. animalis, B. bifidum; Streptococcus such as S. sanguis, S. oxalis, S. mitis, S. thermophilus, S. salivarius; Bacillus such as B. coagulans, B. subtilis, B. laterosporus; Lactococcus such as L. lactis; Enterococcus such as E. faecium; Pediococcus such as P. acidilactici; Propionibacterium suchas P. jensenii, P. freudenreichii; Peptostreptococcus such as P. productus; and Saccharomyces such as S. boulardii.

In various embodiments, the probiotic bacteria of any embodiment are prepared in any manner to be incorporated as a component of a pharmaceutical, food stuff, feed additive, or liquid additive and can be in various forms such as, for example, a liquid state or a dried state including as a powder. In other examples, the probiotic bacteria of various embodiments are dried by air drying method, natural drying method, a spray drying method, a freeze-drying method, or the like. The preparation of the probiotic bacteria can also serve to enhance the properties of the composition including stability. The term “foodstuff” is understood to be any substance or product which in the processed, partially processed, or unprocessed state are intended to be, or reasonably expected to be, ingested by humans. “Foodstuff” can also include drinks, chewing gum, and any substance--including water-intentionally added to the foodstuff during its manufacture, preparation or treatment. The term “feed” is understood to cover all forms of animal food. Foodstuffs can also be used as feeds. The term “pharmaceutical” is understood to cover substances or substance compositions which are intended as agents having properties for curing or for preventing human or animal diseases or which can be used in or on the human or animal body or administered to a human or animal in order to restore, correct or influence either human or animal physiological functions by a pharmacological, immunological or metabolic action, or to produce a medical diagnosis. Pharmaceuticals can be used for non-therapeutic, in particular cosmetic, purposes.

In various embodiments are disclosed pharmaceutical compositions including a compound of any embodiments wherein the pharmaceutical composition is a gel capsule, tablet, pill, lozenge, capsule, microcapsule, liquid, or syrup.

In various embodiments are disclosed orally consumable products including a composition of any embodiment, wherein an orally consumable product is a semi-solid food, solid food, a semi-solid or solid spoonable food, confectionary, drink, or dairy product. The dairy product of various embodiments is ice cream, milk, milk powder, yogurt, kefir, or quark.

In various embodiments are disclosed methods for reducing methionine content of a diet of a subject or enhancing the lifespan of the subject including administering a composition having an amount of probiotic bacteria in a range from about 10³ to about 10¹⁵ cfu per gram of the composition to a subject, wherein a bacterium of the probiotic bacteria, colonizing an intestinal microbiome of the subject, metabolizes methionine at a rate greater than a rate the bacterium synthesizes or recycles methionine when the subject digests a methionine containing substance. In various embodiments, the bacterium substantially always metabolizes methionine at a rate greater than a rate the bacterium synthesizes methionine when the subject digests a methionine containing substance. In other embodiments, the bacterium is unable to synthesize or recycle methionine.

In various embodiments, the rate that the bacterium of the probiotic bacteria, colonizing an intestinal microbiome of the subject, metabolizes methionine is about or is at least about 1%, 2%, 3%, 4%, 5%, 6%, 7%, 8%, 9%, 10%, 20%, 30%, 40%, 50%, 60%, 70%, 80%, 90%, 100%, 100%, 200%, 300%, 400%, 500%, 600%, 700%, 800%, 900%, 1000%, 2000%, 3000%, 4000%, or 5000% greater than the rate the bacterium synthesizes or recycles methionine when the subject digests a methionine containing substance. In various embodiments, the rate is a range between any two percentages listed above. In various embodiments, the rate that the bacterium of the probiotic bacteria, colonizing an intestinal microbiome of the subject, metabolizes methionine is about 5000% or more than the rate the bacterium synthesizes or recycles methionine when the subject digests a methionine containing substance.

In various embodiments, the administering of the composition can be repeated daily for an undetermined period of time. In various embodiments, the probiotic bacteria colonizing the intestinal microbiome of the subject is capable of reducing the methionine content of the diet of the subject by about or by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% generally or within a given time period. In various embodiments, the reduction is a range between any two percentages listed above.

In various embodiments, the administering of the composition is capable of extending the lifespan of the subject by about or at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% as compared to a subject no receiving the composition. In various embodiments, the lifespan extension is a range between any two percentages listed above.

In various embodiments are disclosed methods of preparing the probiotic bacteria of any embodiment. The method of various embodiments can include the step of transfecting or transducing bacteria with a heterologous polynucleotide of any embodiment. In other embodiments, the method can include recombinantly modifying homologous polynucleotides or native promoters to increase or reduce expression of a protein.

The following examples illustrate the various embodiments of the present invention. Those skilled in the art will recognize many variations that are within the spirit of the present invention and scope of the claims.

### EXAMPLE 1

The effect of dietary restriction on Drosophila melanogaster lifespan has been intensely studied. More recently, interest has grown in the microbiome's interaction with its host. This interaction has been shown to influence lifespan but the pathway by which this takes place is not known. Lifespans of flies mono-associated with 41 bacterial strains were studied. Running a metagenome wide association study allowed for the prediction of bacterial genes that were causing lifespan effects. After testing Escherichia coli mutants, the prediction that microbes were influencing Drosophila melanogaster lifespan by altering flux through the transsulfuration pathway was confirmed.

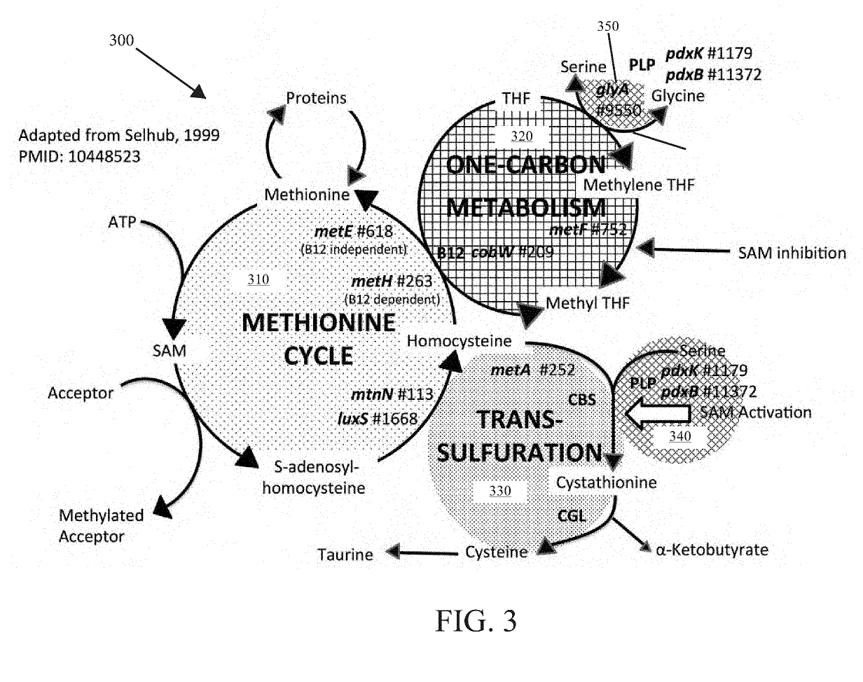

Methionine restriction is an established paradigm for lifespan extension across the tree of life, underscoring its potential as a therapeutic target (ORENTREICH, '93, KOZIEL, '14, LEE, '14). Methionine restriction as a means for lifespan extension is well documented in the fruit fly Drosophila melanogaster, and differential regulation of fruit fly methionine metabolism genes can extend lifespan (KABIL, '11, PARKHITKO, '16). It has also been shown that long-lived Ames dwarf mice lack growth hormone, prolactin, and thyroid stimulating hormone have an enhanced methionine metabolism (UTHUS, '06). In one of the best studied models for aging, fruit fly longevity can be promoted either by restricting the methionine content of the diet, or by differentially expressing genes that decrease the accumulation of methionine-cycle intermediates (TROEN, '07, GRANDISON, '09, KABIL, '11, LEE, '14, OBATA, '15, PARKHITKO, '16). Previous work, primarily in fruit flies and mice, suggests that altering an animal's metabolism to minimize the abundance of methionine cycle metabolites by increasing bacterial transsulfuration flux through transsulfuration decreases the available pool of methionine cycle metabolites (methionine, SAM, SAH, and homocysteine, FIG. 3), supported by the finding that constitutive expression of cystathionine beta synthase (CBS), which drives flux through transsulfuration, extends Drosophila melanogaster lifespan (KABIL, '11). These findings underscore the contributions of model organisms to our understanding of these important biological processes.

Despite our understanding of some of the genes involved, the mechanistic basis for Drosophila responses to dietary and methionine restriction is not fully understood and is complex. For example, the paradox that naturally long-lived flies display increased methionine contents in early life has been explained as an increased flux through the methionine cycle that decreases accumulation of SAM and SAH intermediates (PARKHITKO, '16). This idea is consistent with the explanation that increased SAM catabolism or transsulfuration also extends fruit fly lifespan (KABIL, '11, OBATA, '15). Additionally, some byproducts of methionine metabolism, such as cysteine and cystathionine, restrict lifespan. Finally, we note that in some cases, methionine supplementation is actually helpful for organismal development. These varied findings underscore the complexity of interactions between methionine metabolism and aging.

In addition to dietary and genetic intervations, associated microorganisms (‘microbiota’) are a powerful influence on organismal longevity. In Drosophila melanogaster numerous studies have revealed a positive, neutral, or antagonistic role for the microbiota in animal lifespan. A unifying explanation for the varied influence of bacteria on Drosophila melanogaster lifespan is that the influences of the microbiota are interactive with diet (BRUMMEL, '04, COX, '07, DESHPANDE, '15, YAMADA, '15, YAMADA, '17). For example, when flies are reared on a nutrient poor diet, the microbes may provision nutrients that aid in healthy growth and development, and promote longevity. Conversely, rearing on a nutrient-rich diet may spare the flies of a microbial dependence, and other (currently unknown but possibly pathogenic) influences of the microbiota may prevail. In addition to direct dietary influence on lifespan, there is diet-dependent variation in traits that presumably draw resources away from somatic maintenance, such as immunity (UNCKLESS, '15). Also, the influence of the microbiota on the duration of animal development, a trait that is positively correlated with lifespan in many natural fly populations, is dramatically influenced by changes in absolute and relative nutrient contents in the fly diet (WONG, '14).

Drosophila melanogaster is a well-established model organism for microbiome studies (BRODERICK, '14). As a model for basic and applied aging research, findings in Drosophila are relevant to human studies: human genes that are central to lifespan were first identified in fruit flies, and models for lifespan extension through calorie and methionine restriction were developed in fruit flies (HELFAND, '03, SHAW, '08, BUSHEY, '10). Both mammalian and Drosophila melanogaster lifespan are influenced by the microbiota (BRUMMEL, '04, OTTAVIANI, '11, ZHANG, '13). The fly's digestive tract is similar to humans in physiology and anatomy, making it a key model for studying the microbiome (BRODERICK, '14). Drosophila melanogaster is also an established model for studying the microbiota generally, and flies are readily produced that are reared bacteria-free or with defined microbial communities (including bacterial mutants). Also, host-microbiome interactions first seen in Drosophila have been later observed in mammals, underscoring the broad applicability of many key findings (WONG, '13, ROGERS, '14). Drosophila melanogaster 's simple microbiome also makes them an appealing model. Lab strains typically have between 2-20 bacterial species—being dominated by five or fewer (BRUMMEL, '04, COX, '07, CHANDLER, '11, WONG, '11). Of these five dominant species, the majority are Acetobacter and Lactobacillus species. Furthermore, the microbiota are readily eliminated or inoculated in defined ratios, enabling mechanistic dissection of the varied influences of different microbes on animal traits (CHASTON, '14, NEWELL, '14). Taken together, these tools and a rich history as a model for aging make Drosophila melanogaster an ideal model for understanding host-microbe interactions during aging.

In this work, we investigate the relationship between Drosophila melanogaster, it's lifespan, and it's microbiota and show that bacterial methionine metabolism is important in organismal aging. We also confirmed these predictions by mutant analysis. Finally, we extend these predictions by showing that a microbe engineered to drive flux from the methionine cycle extends organismal lifespan. These results show the influence on Drosophila melanogaster lifespan with a nutrient-rich diet simply by altering their microbes, which is applicable to using such microbes with mammals.

### Methods

### Fly and Bacterial Culture

All experiments were conducted with Drosophila melanogaster. They were obtained and cultured at 25° C. on a 12-hr light-dark cycle. Drosophila melanogaster were fed on a yeast-glucose diet (1 liter H₂O, 100 g inactive brewer's yeast, 100 g glucose, 1.2% agar, 0.84% propionic acid, and 0.08% phosphoric acid) as in our previous work (NEWELL, '14).

The bacterial strains listed in Table 1 were cultured as in our previous work on Glade-specific media: modified MRS medium (mMRS; 1.25% peptone, 0.75% yeast extract, 2% glucose, 0.5% sodium acetate, 0.2% dipotassium hydrogen phosphate, 0.2% triammonium citrate, 0.02% magnesium sulfate heptahydrate, 0.005% manganese sulfate tetrahydrate, 1.2% agar (NEWELL, '14)), potato medium (pot; 0.5% glucose, 1% yeast extract, 1% peptone, 0.8% potato extract), lysogeny broth (LB; 1% tryptone, 0.5% yeast extract, 0.5% sodium choloride), and brain heart infusion broth (BHI). Growth of auxotrophs was confirmed on M9 medium. Escherichia coli were grown at 37° C., and all other strains were grown at 30° C. Transposon insertion- or ectopic expression mutants were cultured with antibiotics: 50 mg/ml kanamycin for Escherichia coli transposon insertion mutants; and 20 μg/ml chlortetracycline for Acetobacter 54 ectopic expression strains. Strains grown in oxic conditions were grown in liquid culture with shaking or with no atmospheric treatment in solid culture. Strains grown under microoxic conditions were grown statically (liquid) or in a sealed, CO₂-flooded chamber (solid).

TABLE 1
Strains

| | | | Oxygen |
|---|---|---|---|
| Identifier | Relevant characteristics | Medium | conditions |
| 7636 | Escherichia coli BW25113, CGSC wild-type; | LB | Oxic |
| | PMIDs: 10829079, 16738554 | | |
| 8422 | CGSC#7636 Δpfs-773::kan; KmR; PMIDs: | LB | Oxic |
| | 10829079, 16738554 | | |
| 9859 | CGSC#7636 ΔpdxB729::kan; KmR; PMIDs: | LB | Oxic |
| | 10829079, 16738554 | | |
| 9920 | CGSC#7636 ΔpdxK747::kan; KmR; PMIDs: | LB | Oxic |
| | 10829079, 16738554 | | |
| 10018 | CGSC#7636 ΔglyA725::kan; KmR; PMIDs: | LB | Oxic |
| | 10829079, 16738554 | | |
| 10100 | CGSC#7636 ΔluxS768::kan; KmR; PMIDs: | LB | Oxic |
| | 10829079, 16738554 | | |
| 10758 | CGSC#7636 ΔmetE774::kan; KmR; PMIDs: | LB | Oxic |
| | 10829079, 16738554 | | |
| 10826 | CGSC#7636 ΔmetF728::kan; KmR; PMIDs: | LB | Oxic |
| | 10829079, 16738554 | | |
| 10856 | CGSC#7636 ΔmetA780::kan; KmR; PMIDs: | LB | Oxic |
| | 10829079, 16738554 | | |
| 10862 | CGSC#7636 ΔmetH786::kan; KmR; PMIDs: | LB | Oxic |
| | 10829079, 16738554 | | |
| 11994 | CGSC#7636 ΔyjiA750::kan; KmR; PMIDs: | LB | Oxic |
| | 10829079, 16738554 | | |
| aanb | Acetobacter aceti NBRC 14818; Accession | mMRS | Oxic |
| | BABW00000000 | | |
| aci5 | Acetobacter sp. DmW_043; Accession | mMRS | Oxic |
| | JOMN00000000 | | |
| afab | Acetobacter fabarum DsW_054-pAH1; Accession | mMRS | Oxic |
| afab- | A. fabarum DsW_054 expressing pAH1; TcR; this | LB | Oxic |
| pAH1 | study | | |
| ain2 | Acetobacter indonesiensis DmW_046; Accession | mMRS | Oxic |
| | JOMP00000000 | | |
| ama3 | Acetobacter malorum DsW_057; Accession | mMRS | Oxic |
| | JOPG00000000 | | |
| amac | Acetobacter malorum DmCS_005; Accession | mMRS | Oxic |
| | JOJU00000000 | | |
| aori | Acetobacter orientalis DmW_045; Accession | mMRS | Oxic |
| | JOMO00000000 | | |
| apa3 | Acetobacter pasteurianus 3P3; Accession | mMRS | Oxic |
| | CADQ00000000c | | |
| apan | Acetobacter pasteurianus NBRC 101655; Accession | mMRS | Oxic |
| | BACF00000000 | | |
| apnb | Acetobacter pasteurianus NBRC 106471 or | mMRS | Oxic |
| | LMG1262t; Accession PRJDA65547 | | |
| apoc | Acetobacter pomorum DmCS_004; Accession | mMRS | Oxic |
| | JOKL00000000 | | |
| atrc | Acetobacter tropicalis DmCS_006; Accession | mMRS | Oxic |
| | JOKM00000000 | | |
| atrn | Acetobacter tropicalis NBRC 101654; Accession | mMRS | Oxic |
| | BABS00000000 | | |
| atro | Acetobacter tropicalis DmW_042; Accession | mMRS | Oxic |
| | JOMM00000000 | | |
| bsub | Bacillus subtilis subsp. subtilis str.168; Accession | LB | Oxic |
| | NC_000964.3 | | |
| ecok | Escherichia coli str. K-12 substr. MG1655; | LB | Oxic |
| | Accession NC_000913.3 | | |
| efav | Enterococcus faecalis V583; Accession | BHI | Oxic |
| | NC_004668.1 | | |
| efog | Enterococcus faecalis OG1RF; Accession | BHI | Oxic |
| | NC_017316.1 | | |
| ehor | Enterobacter hormaechei ATCC 49162; Accession | LB | Oxic |
| | AFHR00000000 | | |
| galb | Gluconobacter sp DsW_056; Accession | Potato | Oxic |
| | JOPF00000000 | | |
| ge5p | Gluconacetobacter europeus 5p3; Accession | Potato | Oxic |
| | CADS00000000 | | |
| gfra | Gluconobacter frateurii NBRC 101659; Accession | Potato | Oxic |
| | BADZ00000000 | | |
| ghan | Gluconacetobacter hansenii ATCC 23769; Accession | Potato | Oxic |
| | ADTV01000000 | | |
| gobo | Gluconacetobacter oboediens 174Bp2; Accession | Potato | Oxic |
| | CADT00000000 | | |
| gxyl | Gluconacetobacter xylinus NBRC 3288; Accession | Potato | Oxic |
| | NC_016037.1 | | |
| lbga | Lactobacillus brevis subsp. gravesensis ATCC | mMRS | Microoxic |
| | 27305; Accession NZ_ACGG01000000 | | |
| lbrc | Lactobacillus brevis DmCS_003; Accession | mMRS | Microoxic |
| | JOKA00000000 | | |
| lbuc | Lactobacillus buchneri NRRLB-30929; Accession | mMRS | Microoxic |
| | ACGG00000000 | | |
| lfal | Leuconostoc fallax KCTC 3537; Accession | mMRS | Microoxic |
| | AEIZ00000000 | | |
| lfer | Lactobacillus fermentum ATCC 14931; Accession | mMRS | Microoxic |
| | ACGI00000000 | | |
| lfrc | Lactobacillus fructivorans DmCS_002; Accession | mMRS | Microoxic |
| | JOJZ00000000 | | |
| lfrk | Lactobacillus fructivorans KCTC 3543; Accession | mMRS | Microoxic |
| | AEQY00000000 | | |
| llac | Lactococcus lactis BPL1; Accession | mMRS | Microoxic |
| lmli | Lactobacillus mali KCTC 3596 = DSM 20444; | mMRS | Microoxic |
| | Accession BACP00000000 | | |
| lplc | Lactobacillus plantarum DmCS_001; Accession | mMRS | Microoxic |
| | JOJT00000000 | | |
| lplw | Lactobacillus plantarum WCFS1; Accession | mMRS | Microoxic |
| | NC_004567.2 | | |
| lrha | Lactobacillus rhamnosus GG; Accession | mMRS | Microoxic |
| | NC_013198.1 | | |
| na | Acetobacter fabarum DsW_054; Accession na | mMRS | Oxic |
| na | Klebsiella variicola ATCC BAA-830; Accession na | LB | Oxic |
| pAH1 | plasmid: pCM62 containing CBS-CGL from K variicola | LB | Oxic |
| | inserted at XbaI and EcoRI sites; TcR; this study | | |
| pbur | Providencia burhodogranariea DSM 19968; | LB | Oxic |
| | Accession AKKL00000000 | | |
| pCM62 | plasmid: Hybrid of pUC19 and pCM51, improved | LB | Oxic |
| | bhr cloning vector (TcR); 11495985 | | |
| pput | Pseudomonas putida F1; Accession NC_009512.1 | LB | Oxic |

### Preparation of Axenic and Gnotobiotic Flies

Flies were reared under axenic or gnotobiotic conditions as in our previous work (KOYLE, '16). Briefly, less than 20 hour-old eggs laid on grape juice agar plates were collected from Drosophila melanogaster and surface sterilized with a 0.6% sodium hypochlorite solution in two 2.5 minute washes. Hypochlorite was washed away by three rinses with sterile water, and 30-60 eggs, qualitatively estimated, were aseptically transferred to sterile diet in a sterile biosafety cabinet. 7.5 ml of sterile yeast-glucose diet (omitting the acid) were inoculated into 50-ml conical tubes, autoclaved, and allowed to cool before transferring sterile eggs. To rear under axenic conditions the eggs were left left undisturbed. To rear with defined bacterial species the sterile eggs were inoculated with 50 μl of bacterial cultures normalized to OD₆₀₀ 0.1. If more than one species was added, the multiple strains were normalized as above and pooled in equal ratios before inoculating the flies with a 50 μl volume of bacteria. For each analysis, we sought to collect data from triplicate vials of flies in each of three separate experiments; however, after 6 separate experiments, some treatments could not be collected at this level of replication and all data were used regardless of replication (after discarding contaminants).

### Lifespan Analysis

Drosophila melanogaster lifespan was measured by recording the number and sex of dead flies and transferring surviving flies to fresh sterile diet every 2-3 days until all flies in a vial were dead. The spent vials were incubated at room temperature (˜22° C.) until eggs laid during the 2-3 day interval grew to adulthood. For every P generation fly vial transferred to fresh diet, one F1 vial from each week of transfers was selected and homogenized to check for bacterial persistence and contamination during transfer. A pool of five mixed sex flies from each vial was homogenized in 125 μl homogenization buffer (10 mM Tris, pH 8, 1 mM EDTA, 0.1% Triton X-100 as in (CHASTON, '14)) with 125 μl Lysing Matrix D ceramic beads (MP Biomedicals 116540434) by shaking for 30-60 s at 4.0 M/S in a FastPrep-24, dilution plated onto mMRS medium, incubated under oxic and microoxic conditions, and visually inspected by colony morphology to confirm strain identity. If at least 200 CFU fly⁻¹ of the expected bacterial strain were detected, the strain was deemed ‘present’ (see below for incorporation into statistical models). If at least 200 CFU fly⁻¹ of an unexpected bacterial species were detected in 2 consecutive weeks, the vial was deemed contaminated. Differences between Acetobacter strains could usually not be determined by colony morphology, so Acetobacter contamination of other Acetobacter strains cannot be ruled out.

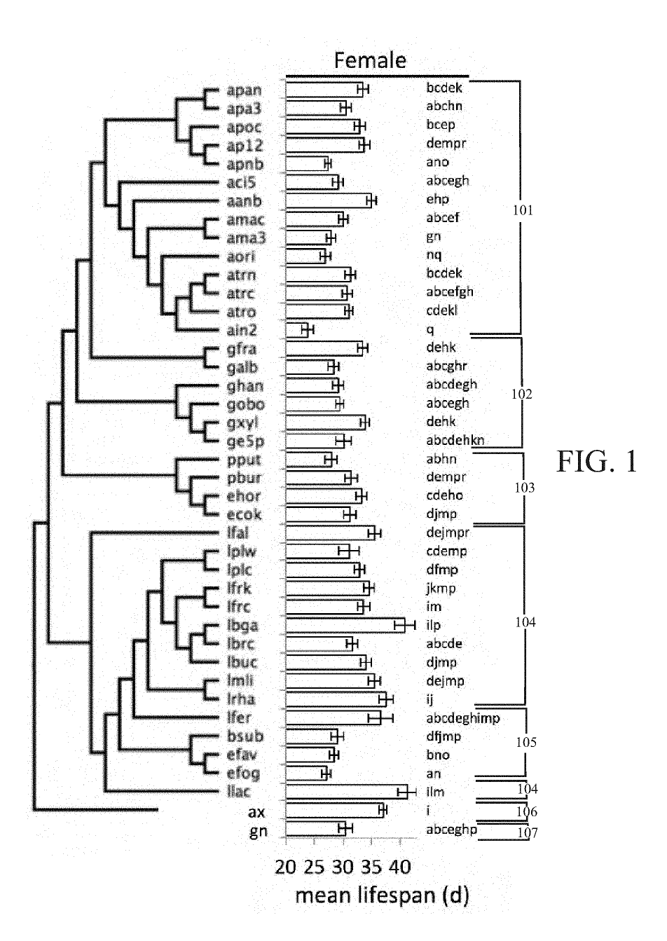

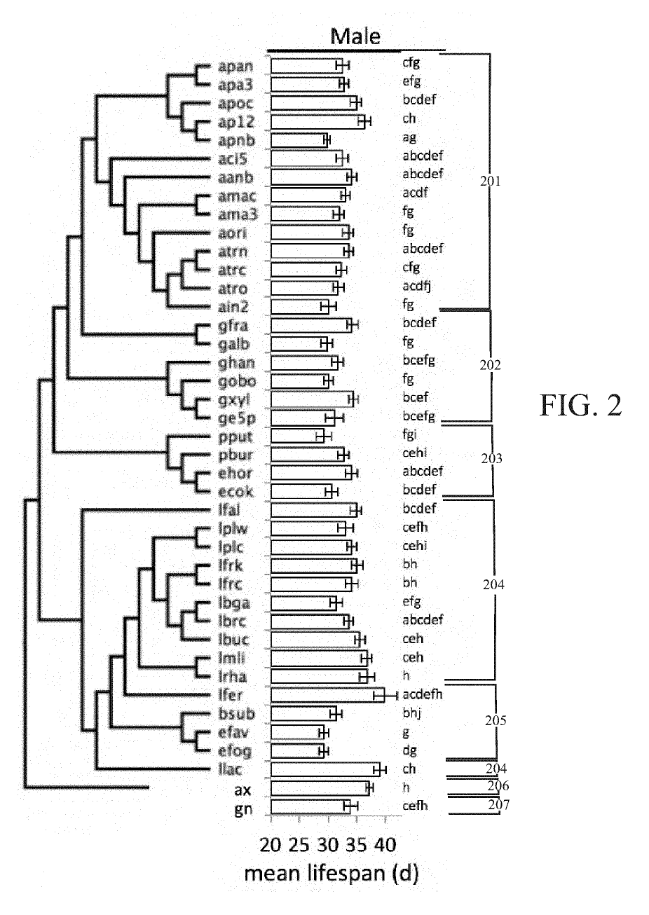

The lifespan analysis for flies bearing Escherichia coli mutants was conducted exactly as described above with one exception: because Escherichia coli persisted poorly in flies during the first lifespan experiment (see FIGS. 1 and 2), each P generation fly vial was re-inoculated with the corresponding Escherichia coli mutant for the second, third, and fourth weeks after eclosion. Auxotrophs were distinguished by their ability to grow on M9 medium supplemented with the auxotrophic nutrient.

Differences in fly lifespan with bacterial treatments were determined by a left- and right-censored Cox mixed-effects survival model in R (THERNEAU, '12, THERNEAU, '14) to account for differences in bacterial persistence in the flies. All flies entered the experiment at the time of egg transfer to sterile diet, and bacterial presence was indicated as a fixed effect with the value of “1”. If the F1 bacterial vials were uncontaminated and no bacteria were detected in two consecutive weeks, the bacteria-associated flies were marked to exit the experiment at the last day bacteria were detected in F1 vials (right-censored); at the same day, the same flies entered the experiment (left-censored) with a bacterial presence value of “0”. For F1 vials that were contaminated in 2 consecutive weeks, flies from the P vials were marked as leaving the experiment on the latest day that no contamination was detected. If the flies were contaminated from the first transfer onward, the entire vial was excluded from the analysis.

### Meta-Genome Wide Association

To predict bacterial genes that influenced lifespan, a meta-genome wide association (MGWA) approach was used, as in our previous work (CHASTON, '14). Amino acid sequences from each bacterial species used in the monoassociation experiments were obtained by whole-genome sequencing or from Genbank, and were clustered in orthologous groups (OGs) using a local installation of the OrthoMCL software with an inflation factor of 1.5. MGWA was performed using the MGWAR R package (SEXTON, '18). Differences in lifespan of the flies, relative to axenic flies, were determined by the right- and left-censored Cox mixed effects model described above. OGs were ranked according to p-value with an FDR-corrected p-value of ≤0.01 considered significant.

To identify functional categories that were enriched among the significantly-associated COGs a KEGG enrichment analysis was performed. KEGG categories were assigned to a representative sequence from each OG using BlastKOALA. The KEGG pathway assignments to each OG were retrieved in KEGG PATHWAY, and chi-square tests were performed to test for pathways that were enriched in the top 170 OGs relative to all 12980 clustered OGs. P-values were false-discovery rate (FDR) corrected for multiple tests. Chi-square tests and FDR correction were performed in R (CORE TEAM, '16).

The phylogenetic tree shown in FIGS. 1 and 2 were built by manually extracting 16S rRNA gene sequences from the nucleotide sequences of each sequenced genome, aligning with the EMBL-EBI online MUSCLE tool (www.ebi.ac.uk/Tools/msa/muscle/), manually trimming to an aligned (with gaps) partial 16S rRNA sequence length of 1348 bp, and rerunning the alignment with default ClustalW parameters. The phylogenetic tree was downloaded and formatted in FigTree v 1.4.3 (tree.bio.ed.ac.uk/software/figtree/). A 16S rRNA gene sequence for Halobacterium jilantaiense JCM 13558, Accession NR_113425 was used as an outgroup.

Expression of Klebsiella variicola CBS::CBL in A. fabarum DsW_054

Plasmid pAH1 was constructed by insertion of the 2.5 Kb CBS-CBL Klebsiella variicola operon [SEQ ID NO: 11] into expression vector pCM62. Genomic DNA was isolated from 1.5 ml of K variicola culture using the DNeasy Blood and Tissue Kit (Qiagen). The CBS-CGL fragment was amplified from with Pfx polymerase (Thermo Fisher Scientific) and the CBSCGL-xbaI-for (5′-NNNNtctagaATGTCACTGTTTCATTCC-3′ [SEQ ID NO: 12]; restriction enzymes in lowercase here and hereafter) and CGL-ecoRI-rev (5′-NNNNgaattcCGGAATAATCACTCCTCC-3′ [SEQ ID NO: 13]). The pCM62 plasmid was digested using Xbal and EcoRI (New England Biolabs) according to manufacturer's recommendations, and the CBS-CGL fragment was ligated into the plasmid using T4 DNA Ligase (New England Biolabs). The assembled pAH1 plasmids were then electroporated into Escherichia coli S17 λ-Pir, and selected on LB plates containing chlortetracycline at a concentration of 5 ng/mL. Successful cloning was initially screen by PCR across the polylinker, selecting for colonies with the 2.5 kB insert, and the sequence of the insert was verified in its entirety by Sanger sequencing at the BYU DNA Sequencing center using primers cbscgl-500 (5′-ACCACTTATTCAGCGAACC-3′ [SEQ ID NO: 14]), CBS_CGL-1000 (5′-GGAGGAACGATAATCGAAG-3′ [SEQ ID NO: 15]), CBS_CGL_1500 (5′-GATCCTGGCTGGTCGAAG-3′ [SEQ ID NO: 16]), CBS_CGL-2000 (5′-CCAACGCTTCTCCCTGCC-3′[ SEQ ID NO: 17]), CBS_CGL_2500 (5′-TGGATAAGGACAGTCACC-3′ [SEQ ID NO: 18]), and CBS_CGL_3000 (5′-GAAGTGGAGCGAGTCTGG-3′ [SEQ ID NO: 19]). This created plasmid pAH1 pAH1 was conjugated into Acetobacter fabarum DsW_054 as described previously, with minor changes (WHITE, '18). Briefly, S17 λ-Pir Escherichia coli containing pAH1 were grown overnight at 37° C. in LB-chlortetracycline, and A. fabarum DsW_054 was grown at 30° C. in potato medium. 500 μl of cells from each culture were centrifuged, the supernatants discarded, and each pellet was resuspended in 50 μl of potato medium. 50 μl of each cultured was mixed in microcentrifuge tube and incubated at 30° C. for 16 hours. The 100 μl mixture was then plated onto YPG medium (1.5% agar, 1% glycerol, 0.5% peptone, 0.5% yeast extract, 0.2% acetic acid and 20 mg/L chlortetracycline (CHASTON, '14). YPG plates containing transformed A. fabarum DsW_054 were incubated for 72 hours, after which colony PCR and plasmid isolation were performed to confirm the presence of the plasmid.

### Results

### Effects of Microbial Strains and Their Persistence on Lifespan

To study the effects that different bacterial species had on the lifespan of Drosophila melanogaster, we monoassociated the flies with different bacterial strains and measured fly lifespan. As controls we measured the lifespans of bacteria free flies and flies that were associated with a representative five-species bacterial community, as in our previous work (NEWELL, '14).

FIGS. 1 and 2 show the bacterial effects on the mean lifespan of female and male Drosophila melanogaster, where lifespans were measured in flies monoassociated with the bacteria from different taxonomical groups. The reference numbers in FIGS. 1 and 2 highlight different taxonomical groups, where: reference 101 and 201 highlight bacteria strains from the Acetobacter genus; reference 102 and 202 highlight bacteria strains from the Gluconacetobacter genus; reference 103 and 203 highlight bacteria strains from the γ-proteobacteria class; reference 105 and 205 highlight bacteria strains from the Lactobacilli genus; reference 105 and 205 highlight bacteria strains are non-lactobacillus Firmicutes; reference 106 and 206 are the bacteria free controls, where “ax” represents axenic; and reference 107 and 207 represent controls, where “gn” represents gnobiotic. The abbreviations listed in FIGS. 1 and 2 correlate to the identifiers in Table 1 and different letters by the bars represent statistically significant differences between treatments.

As shown in FIGS. 1 and 2, microbial presence generally decreased fruit fly lifespan relative to micro-organism free flies, with variation in lifespan effects within and between bacterial species. For example, the Acetobacteraceae tended to confer a shorter lifespan on the flies than Lactobacillus species, but some species did not follow this trend. Altogether, these data show that the microbial strains present in the flies had a significant impact on the lifespan of the flies.

Variation in lifespan effects of the different bacteria was specific for lifespan and was likely tied to specific bacterial functions. For example, the variation could not be attributed exclusively to developmental delays since lifespan varied over many days and microbial influence on development varies across less than 24 hours on the rich diet used in our experiments (see, e.g. (CHASTON, '14)). The effect of bacterial persistence was also independent of bacterial identity and, presumably, function. Frequent transfer of flies to fresh diets, as is necessary in lifespan experiments, can cause loss of the associated microbial communities in Drosophila (BLUM, '13). To test how bacteria persisted in the flies throughout our experiment and if it impacted fly lifespan, we measured the period over which bacteria were detected in Fl generation flies that hatched on spent media after P generation flies were transferred to fresh diet. There was wide variation in bacterial persistence in the flies with one Lactobacillus strain lost from flies after the first transfer, and numerous Acetobacter strains detectable in spent P generation vials for the entire experiment. When the period during which bacteria were transferred to spent P generation vials was included in a left- and right-censored survival model, the effect was significant. Additionally, there was a positive correlation between bacterial persistence and lifespan in Proteobacteria-associated flies, but not in flies bearing Firmicutes isolates or when the taxonomic groups were considered together, suggesting that the period during which bacteria were associated with the flies was associated with conspecific Acetobacter functions but was not a directly-influencing lifespan factor. Because the sampling of microbiota abundance is destructive, we were unable to determine if microbial load over time also contributed to the species-specific effects. Regardless, these findings together suggest that while the period during which bacteria are present can influence the lifespan effect, it is the identity of the persisting microbe to which the effect must be attributed. Given the varied influence of bacteria on Drosophila lifespan it may be possible that this finding will not be consistent in flies reared on other, possibly less nutritional, diets, where increases in bacterial load may be correlated with increased nutritional provisioning.

Identifying Bacterial Genes with Effects on Lifespan

To identify possible bacterial genes that influence Drosophila melanogaster lifespan, we performed a meta-genome wide association study. As shown in Table 2, 12 of the 12,980 OGs significantly reduced or extended lifespan. A hit was considered significant if the P value with the Bonferroni correction was less than or equal to 0.05. Within the top most significant OGs, genes involved in vitamin B2, vitamin B12, and methionine metabolism were detected. Visual inspection of other highly ranked genes revealed many genes with small p-values before correction for multiple tests that were associated with functions in vitamin B5, B6, and folate metabolism. To focus our study on classes of bacterial functions that were predicted to influence lifespan we performed a KEGG enrichment analysis, using all OGs with a false-discovery rate corrected p-value<0.01 (170 OGs total). As shown in Tables 2 and 3, two KEGG categories were significantly enriched among the top 170 OGs in the MGWA: glucagon signaling; and cysteine and methionine metabolism. Since glucagon signaling is an animal pathway and bacteria only bear homology to scattered genes in the pathway, we focused our remaining efforts on testing the hypothesis that microbial cysteine and methionine metabolism influences Drosophila melanogaster lifespan.

TABLE 2
MGWA predictions. Abbreviations: Acetobacter spp. (Ac), Gluconacetobacter spp.
(G), Lactobacillaceae (Lac), Gammaproteobacteria (Gamma), other abbreviations as in Table 1.

| | Bonferroni | | | | |
|---|---|---|---|---|---|
| | corrected_ | mean_OG | mean_OG | | rep_annotation |
| OG_ID | p-val | Contain | Lack | OG present in | (KEGG ID) |
| stl00615 | 7.37E−06 | 29.4 | 34.7 | 12 Ac, 5 G., 2 | transport system |
| | | | | Lac, 2 gamma, | permease (K02015) |
| | | | | efav, efog | |
| stl00806 | 9.93E−05 | 34.1 | 29.2 | 9 lac, 2 Gamma, | membrane protein |
| | | | | bsub | |
| stl00883, | 1.32E−03 | 30.0 | 33.8 | 12 Ac, efav, | ankyrin (K06867); |
| stl00888, | | | | efog, 4 gamma, | Putative metal |
| stl01007 | | | | 5 G | chaperone (Zn); |
| | | | | | Deoxyribodipyrimidine |
| | | | | | photolyase |
| | | | | | (K01669) |
| stl02563, | 1.32E−03 | 33.8 | 30.0 | 9 lac, bsub | pheromone precursor |
| stl02568, | | | | | lipoprotein CamS; |
| stl02572, | | | | | Cell division FtsL; |
| stl02573 | | | | | RibT (K02859); |
| | | | | | hypothetical |
| stl02493, | 2.44E−03 | 33.8 | 30.1 | 8 lac, bsub | membrane protein; |
| stl02927, | | | | | hypothetical; Septum |
| stl02930, | | | | | site-determining MinC |
| stl03103, | | | | | (K03610); trypsin-like |
| stl01237 | | | | | serine protease; MFS |
| | | | | | family transporter |
| stl01019 | 5.11E−03 | 29.5 | 34.2 | 11 Ac, 3 | ribonuclease PH |
| | | | | gamma, bsub, | |
| | | | | efav, efog | |
| stl02777 | 1.20E−02 | 34.2 | 29.6 | 7 lac, bsub, 2 | ribosomal-protein- |
| | | | | Gamma | L7/L12-serine |
| | | | | | acetyltransferase |
| | | | | | (K03817) |
| stl02172 | 1.48E−02 | 33.6 | 30.1 | 8 lac, ghan, bsub | NAD-dependent |
| | | | | | epimerase/dehydratase |
| stl03062 | 1.99E−02 | 33.3 | 30.4 | 7 lac, bsub, pbur | ABC transporter metal |
| | | | | | ion transporter |
| | | | | | periplasmic |
| | | | | | component/surface |
| | | | | | antigen (K02073) |
| stl02146, | 2.05E−02 | 34.2 | 29.5 | 9 lac, ecok, ehor | rRNA (cytosine-C(5)-)- |
| stl00713 | | | | | methyltransferase |
| | | | | | RsmF, Hydrolase |
| | | | | | (HAD superfamily) |
| | | | | | (K07757) |
| stl02072, | 3.06E−02 | 34.2 | 30.2 | 8 lac | hypothetical protein, |
| stl01315 | | | | | L-2- |
| | | | | | hydroxyisocaproate |
| | | | | | dehydrogenase |
| | | | | | (K00016) |
| stl01428 | 3.17E−02 | 34.1 | 29.6 | 6 lac, 3 gamma, | NAD(P)H |
| | | | | bsub | dehydrogenase |
| | | | | | (quinone) |
| stl00675 | 3.65E−02 | 29.3 | 33.6 | 12 Ac, 5 G, | dipeptide ABC |
| | | | | pbur, efav, efog | transporter substrate- |
| | | | | | binding protein |
| | | | | | (K02035) |
| stl00558 | 3.72E−02 | 33.2 | 29.5 | 9 lac, 3 gamma, | S-methylmethionine |
| | | | | bsub | transport protein |
| | | | | | (K11733) |
| stl02793, | 3.91E−02 | 33.8 | 30.3 | 7 lac, bsub | sodium/hydrogen |
| stl03291 | | | | | exchanger (K03455); |
| | | | | | transport protein |
| | | | | | (K06994) |

TABLE 3
KEGG enrichment analysis of significant MGWAS predictions

| | Top | All genes |
|---|---|---|
| | predictions | (4828 |
| Annotation | (91 genes) | genes) |
| Cysteine and methionine metabolism* | 7.7% | 1.5% |
| Glucagon signaling pathway * | 3.3% | 0.3% |
| ABC transporters | 3.3% | 5.8% |
| Folate biosynthesis | 3.3% | 0.7% |
| Purine metabolism | 6.6% | 2.2% |
| Sulfur relay system | 2.2% | 0.5% |
| Pyrimidine metabolism | 4.4% | 1.6% |
| Glycerolipid metabolism | 2.2% | 0.6% |
| Pyruvate metabolism | 4.4% | 1.8% |
| Quorum sensing | 3.3% | 3.1% |
| Alanine, aspartate and glutamate metabolism | 2.2% | 0.7% |
| Glutathione metabolism | 2.2% | 0.7% |
| Carbon metabolism | 4.4% | 3.7% |
| Biosynthesis of amino acids | 7.7% | 4.1% |
| Glycerophospholipid metabolism | 2.2% | 0.9% |
| Biosynthesis of antibiotics | 9.9% | 6.8% |
| Sulfur metabolism | 2.2% | 1.0% |
| Biosynthesis of secondary metabolites | 15.4% | 9.2% |
| Glycolysis/Gluconeogenesis | 3.3% | 1.7% |
| Glycine, serine and threonine metabolism | 2.2% | 1.6% |
| Arginine and proline metabolism | 2.2% | 1.2% |

### Testing Identified Bacterial Genes for Effect on Lifespan

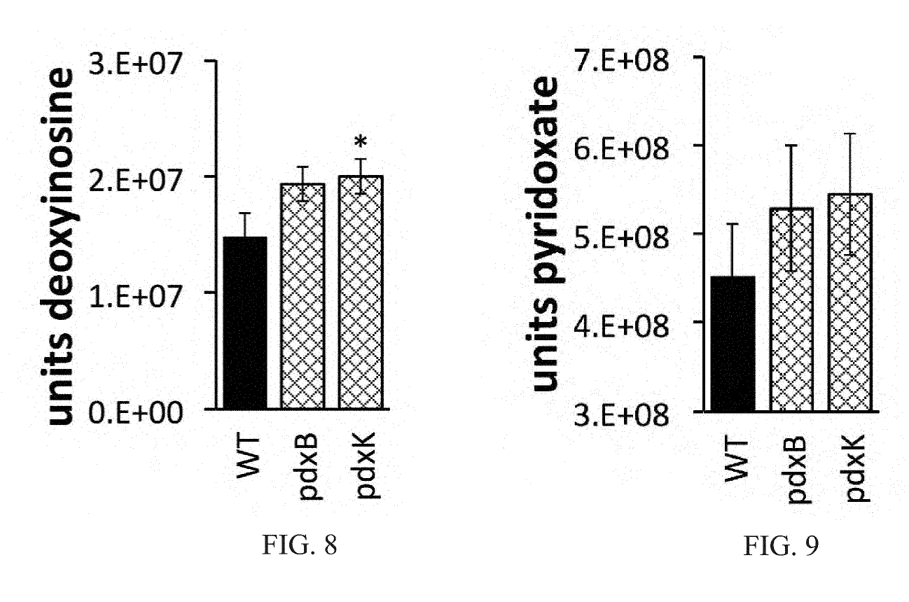

FIGS. 3-10 highlight the influence of bacterial methionine cycle mutants on Drosophila melanogaster lifespan and metabolites.

FIG. 3 shows a diagram of the methionine cycle 300, including the methionine cycle, one carbon metabolism, transsulfuration, and vitamin B6-dependent interconversion of glycine and serine. Reference 310 highlights the methionine pathway. Reference 320 highlights the vitamin B12 dependent pathway for converting homocysteine to methionine. Reference 330 highlights the transsulfuration pathway. Reference 340 and 350 highlight parts of the pathway requiring vitamin B6 (pyridoxal phosphate or PLP). Gene names that were tested with Escherichia coli mutants are placed in the area of the pathway they effect. FIG. 3 was adapted from Selhub, Jacob. “Homocysteine metabolism.” Annual review of nutrition 19.1 (1999): 217-246.

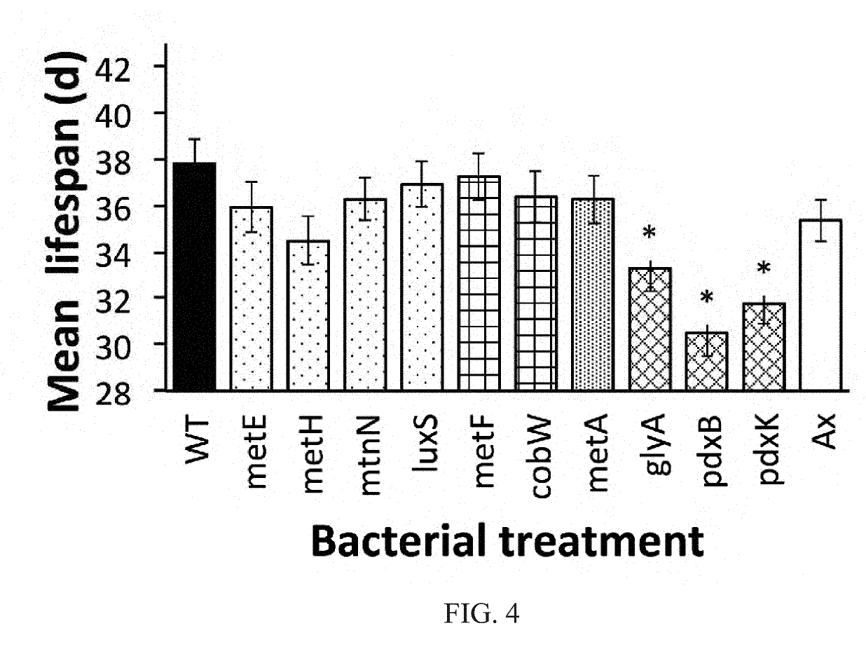

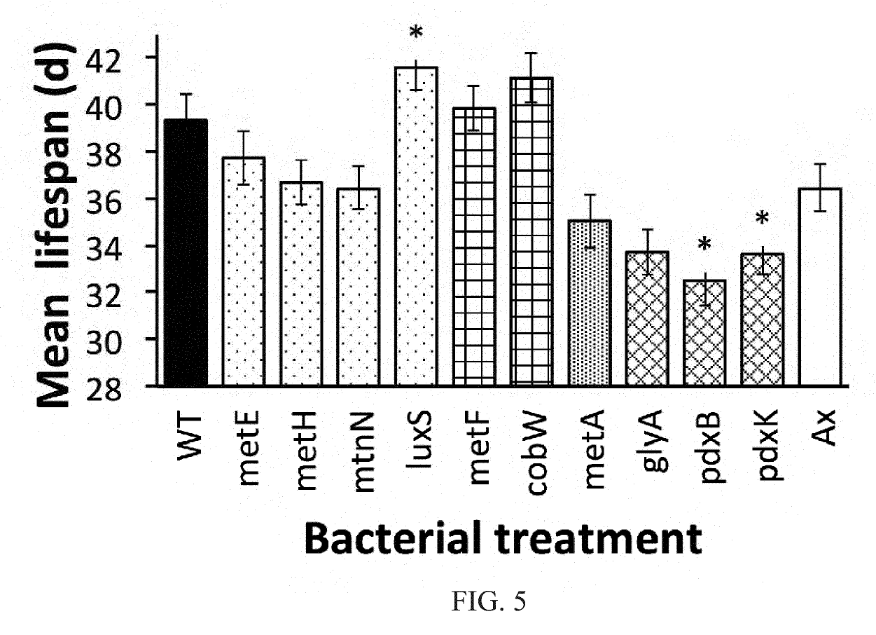

FIGS. 4 and 5 show average lifespans of female and male Drosophila melanogaster monoassociated with Escherichia coli mutant strains. The patterns of the bars correspond to patterns in the parts 310,320,330,340,350 of the methionine cycle 300 shown in FIG. 3. The indication “*” signifies differences for mutants relative to wild-type bacteria, determined by a Cox mixed effects survival model (p<0.05). The patterns of the bars correspond to patterns in the portion 310,320,330,340,350 of the methionine cycle 300 shown in FIG. 3. The following abbreviation are understood to mean: “metE” is 5-methyltetrahydropteroyltriglutamate-homocysteine methyltransferase; “metH” is methionine synthase; “mtnN” is 5′-methylthioadenosine/S-adenosylhomocysteine nucleosidase or adenosylhomocysteine nucleosidase; “luxS” is S-ribosylhomocysteine lyase; “metF” is 5,10-methylenetetrahydrofolate reductase; “cobW” is cobalamin synthase W domain-containing protein 1; “metA” is homoserine O-succinyltransferase; “glyA” is serine hydroxymethyltransferase; “pdxB” is Erythronate-4-phosphate dehydrogenase; and “pdxK” is Pyridoxine/pyridoxal/pyridoxamine kinase.

As a first test of the MGWA prediction that bacterial cysteine and methionine metabolism influence Drosophila melanogaster lifespan we performed a mutant analysis. We measured lifespan in Drosophila melanogaster that were monoassociated with Escherichia coli strains bearing mutations in methionine metabolism genes. The results from the measurements are shown in FIGS. 4 and 5, where mutations that alter flux through these pathways 300 alter fly lifespan. To ensure continued exposure of the flies to Escherichia coli bacteria, a different strain of which persisted poorly during thrice-weekly fly transfers to fresh diets in our first set of experiments (data not shown), fly vials were reinoculated with the bacteria once per week. On a nutritionally rich diet, associated microbes normally shorten Drosophila melanogaster lifespan (data not shown). As shown in FIGS. 4 and 5, the luxS mutant was the only mutant that significantly extended fruit fly lifespan, and the effect was only detectable in male flies. Conversely as shown in FIGS. 4 and 5, two bacterial mutants related to vitamin B6 biosynthesis—pdxB, pdxK—each significantly shortened fly lifespan relative to wild-type Escherichia coli strains in male and female flies, and a mutation in the glyA gene, which is involved in the vitamin B6-dependent conversion of glycine to serine, shortened female lifespan. Manual examination of Kaplan-Meier plots suggested that many of the mutants extended lifespan of long-lived flies. We confirmed this by comparing the lifespans of wild-type versus mutant bacteria-associated flies that lived longer than 47 days post egg-deposition. Under these selective criteria, four and six of the tested mutants significantly extended fruit fly lifespan in female and male flies, respectively, including three mutants that extended lifespan in both males and females: metA, glyA, and luxS. Together, these findings reveal that disruption of normal methionine metabolism in associated bacteria can either reduce or extend fruit fly lifespan.

### Metabolomic Effects of Mutant Species on Flies

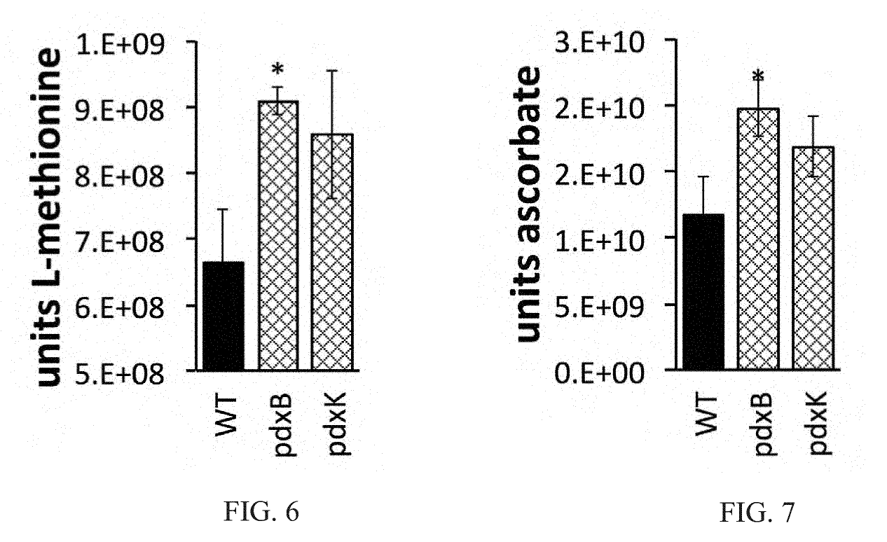

FIGS. 6-9 show differences in metabolite levels in flies reared with a wild-type bacteria or flies reared with pdxB or pdxK mutants, where the differences are calculated by a Wilcoxon test (*p<0.05). The patterns of the bars correspond to patterns in parts 340,350 of the methionine cycle 300 shown in FIG. 3.

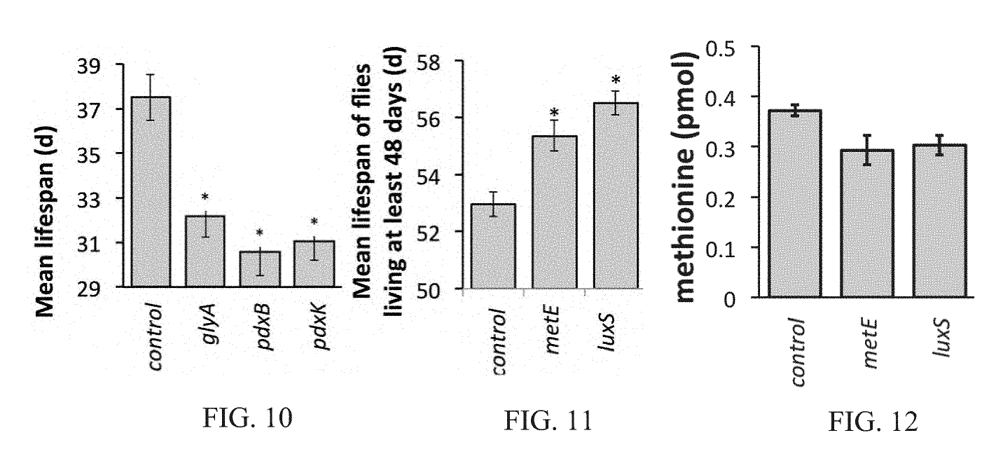

FIG. 10 shows mean lifespan differences in flies reared wild-type bacteria or flies reared with pdxB, pdxK, or glyA mutants, where mutations in bacterial glyA, pdxB, and pdxK genes increased flux through methionine pathway and shorten fruit fly lifespan.

FIG. 11 shows mean lifespan differences in flies reared wild-type bacteria or flies reared with metE or luxS mutants and living at least 48 days, where Mutations in methionine pathway genes metE and luxS that decrease methionine production, increase lifepsan in old (>48 day old) flies.

FIG. 12 shows an metobolomic analysis of diets showing differences in S-ribosylhomocysteine abundance of flies reared with pdxB, pdxK, or luxS mutants as compared to their wild-type counterparts, where Mutations in metE and luxS lower the methionine content of D. melanogster diet by ˜25% as measured by high performance liquid chromatography (HPLC).

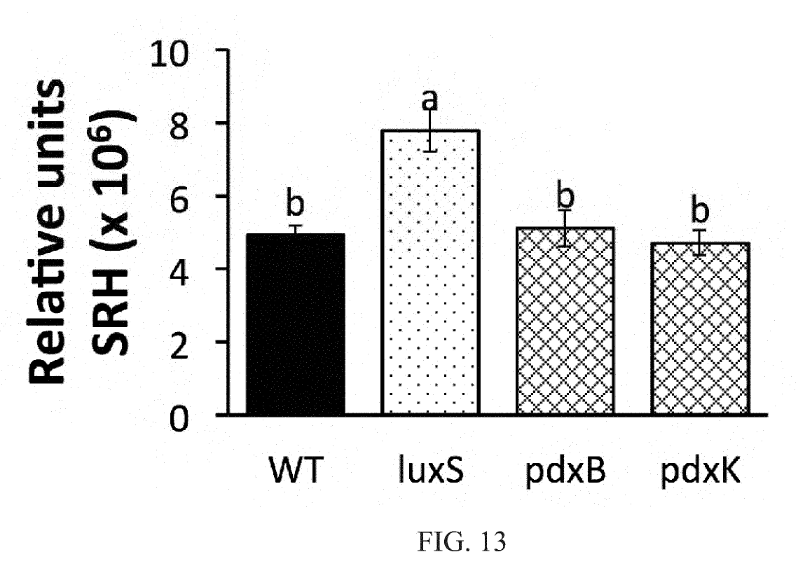

FIG. 13 shows differences in S-ribosyl homocysteine (SRH) abundance in the diet were determined by targeted metabolomics, where different letters represent significant differences between treatments at p<0.05 as calculated by a Dunn test. The patterns of the bars correspond to patterns in parts 310,340,350 of the methionine cycle 300 shown in FIG. 3.

On a nutritionally rich diet, associated microbes normally shorten Drosophila melanogaster lifespan (data not shown). To better understand the molecular basis for these effects we performed a screen to predict specific bacterial gene mutations that shorten or extend fruit fly lifespan, and validated predictions in fruit flies monoassociated with bacteria bearing mutations in the predicted genes. The mutant analysis identified mutations in the methionine cycle and transsulfuration that shorten (FIG. 10; vitamin B6 and glycine metabolism, cofactors for CBS in transsulfuration) or extend lifespan (FIG. 11, methionine cycle). The data supports the understanding that bacterial methionine metabolism normally supplements an animal's methionine intake, and that bacteria that contribute more or less than the normal dietary supply of methionine have consequent influence on lifespan of the animal. Further experiments with some of the bacterial mutants confirmed that the bacteria influence methionine content of diets (FIG. 13). Together, these data supported the idea that bacterial methionine metabolism could influence fruit fly lifespan.

To begin to understand why the pdxB and pdxK mutants shortened Drosophila lifespan, whereas the luxS mutants promoted longevity, we performed a global metabolomic analysis of flies or diets that had been monoassociated with the different mutants. When we compared the metabolomes of the flies bearing mutant versus wild-type Escherichia coli strains, we detected only 3 metabolites with significantly different abundant in the pdxB (methionine, ascorbate) and pcbcK (deoxyinosine) flies as shown in FIGS. 6-9 and Table 4.

TABLE 4

| Metabolite identity | ratio pdxK:WT | ratio pdxB:WT |
|---|---|---|
| Pyruvate | 1.1791 | 1.4044 |
| Alanine.Sarcosine | 1.0963 | 1.1635 |
| Serine | 1.0183 | 1.0912 |
| Histamine | 1.1038 | 1.0914 |
| Uracil | 1.0218 | 1.0930 |
| Proline | 1.1648 | 1.2239 |
| Fumarate | 1.0645 | 1.2367 |
| Indole | 1.2313 | 1.1961 |
| Valine | 1.3471 | 1.4834 |
| Succinate.Methylmalonate | 1.0387 | 1.0944 |
| Homoserine.Threonine | 1.1778 | 1.3152 |
| Cysteine | 1.1599 | 1.2148 |
| Nicotinate | 0.9656 | 0.9965 |
| Taurine | 1.0739 | 1.1046 |
| Thymine | 1.0874 | 1.2012 |
| Leucine.Isoleucine | 1.3025 | 1.4108 |
| Asparagine | 1.1035 | 1.1543 |
| Ornithine | 1.0654 | 1.1412 |
| Aspartate | 1.0344 | 1.1790 |
| Malate | 1.0575 | 1.2249 |
| Hypoxanthine | 0.8139 | 0.9046 |
| Histidinol | 1.0751 | 1.2525 |
| X2.Oxoglutaric.acid | 1.2020 | 1.1943 |
| Glutamine | 1.1528 | 1.2245 |
| Lysine | 1.0670 | 1.1409 |
| Glutamate | 1.1691 | 1.2065 |
| O.Acetyl.L.serine | 1.1700 | 1.2123 |
| X2.Hydroxy.2.methylsuccinate | 1.0614 | 1.0240 |
| L.Methionine | 1.2916 | 1.3689* |
| Guanine | 0.8015 | 0.5693 |
| Xanthine | 1.3050 | 1.2584 |
| Dopamine | 1.1895 | 1.2360 |
| Histidine | 1.0296 | 1.1422 |
| Orotate | 0.9778 | 1.0507 |
| Allantoin | 1.2058 | 1.0090 |
| Indole.3.carboxylate | 0.9403 | 0.9533 |
| Phenylalanine | 1.2184 | 1.2881 |
| Uric.acid | 1.1224 | 1.2713 |
| Cysteate | 0.8104 | 0.9146 |
| X1.Methylhistidine | 0.9139 | 1.0004 |
| Sulfolactate | 0.8472 | 0.8600 |
| D.Glyceraldehdye.3.phosphate | 0.9904 | 1.0397 |
| Glycerone.phosphate | 0.9898 | 1.0387 |
| sn.Glycerol.3.phosphate | 0.9505 | 1.0071 |
| Aconitate | 1.0425 | 1.0951 |
| Arginine | 1.0769 | 1.1584 |
| Citrulline | 1.1333 | 1.1887 |
| Ascorbate | 1.4413 | 1.6923* |
| N.Carbamoyl.L.aspartate | 1.0875 | 0.9495 |
| Glucosamine | 1.1025 | 1.2432 |
| myo.Inositol | 1.0901 | 1.2909 |
| Tyrosine | 1.1167 | 1.2040 |
| X4.Pyridoxate | 1.2070 | 1.1708 |
| X3.Phosphoserine | 0.7271 | 0.8495 |
| N.Acetylglutamine | 1.1228 | 1.2455 |
| Acetyllysine | 1.1451 | 1.1080 |
| Kynurenic.acid | 1.1746 | 0.9630 |
| N.Acetylglutamate | 1.0409 | 1.0813 |
| X2.Dehydro.D.gluconate | 0.7506 | 0.1030 |
| D.Gluconate | 0.7934 | 0.5594 |
| Tryptophan | 1.0542 | 1.1332 |
| Xanthurenic.acid | 0.9793 | 0.9846 |
| Kynurenine | 1.0468 | 1.1780 |
| Cystathionine | 1.1710 | 1.2150 |
| Ribose.phosphate | 0.9931 | 1.0711 |
| Cystine | 0.9687 | 1.0942 |
| Uridine | 1.0886 | 1.0886 |
| Deoxyinosine | 1.3599* | 1.3135 |
| Glucosamine.phosphate | 1.0460 | 1.2631 |
| Glucose.1.phosphate | 1.1521 | 1.2213 |
| Glucose.6.phosphate | 1.1535 | 1.2148 |
| S.Ribosyl.L.homocysteine | 0.9523 | 1.2227 |
| Adenosine | 1.2524 | 1.1956 |
| Inosine | 1.0422 | 1.0505 |
| X6.Phospho.D.gluconate | 0.9740 | 1.1224 |
| X1.Methyladenosine | 1.0472 | 1.1300 |
| Guanosine | 1.1757 | 1.2408 |
| Xanthosine | 1.0407 | 1.1591 |
| Sedoheptulose.1.7.phosphate | 1.1055 | 1.1655 |
| S.Methyl.5..thioadenosine | 1.1149 | 0.6195 |
| N.Acetylglucosamine.1.6.phosphate | 1.0638 | 1.1691 |
| Glutathione | 1.2445 | 1.2914 |
| CMP | 1.1986 | 1.2766 |
| UMP | 1.2431 | 1.3420 |
| Trehalose.Sucrose.Cellobiose | 0.9459 | 1.0591 |
| AMP.dGMP | 1.0982 | 1.1638 |
| IMP | 1.0425 | 1.0938 |
| GMP | 1.0957 | 1.1583 |
| Riboflavin | 1.0080 | 1.2677 |
| S.Adenosyl.L.homocysteine | 0.9774 | 1.2794 |
| Trehalose.6.phosphate | 0.9217 | 1.0886 |
| CDP.ethanolamine | 1.3214 | 1.5259 |
| UDP.glucose | 1.1621 | 1.1663 |
| UDP.N.acetylglucosamine | 1.0850 | 1.0872 |
| Glutathione.disulfide | 1.0803 | 1.1287 |
| NAD. | 1.1374 | 1.1878 |
| NADH | 0.9625 | 0.9279 |
| FAD | 1.1719 | 1.3485 |
| N.Acetylornithine | 1.1143 | 1.2233 |
| Pyroglutamic.acid | 1.0001 | 1.0427 |
| Allantoate | 4.9465 | 0.9116 |
| Xylitol.5.phosphate | 0.7958 | 0.6542 |
| Ophthalmate | 1.1446 | 1.5347 |

Further, a transcriptomic analysis of flies bearing pdxK mutant or wild-type bacteria also revealed few differences (data not shown). Taken together, these findings suggest that the dramatic reduction in fly lifespan was not tied to generalized malaise in the flies; but to the small number of specific changes we detected. Similarly, a metabolomic analysis of diets on which flies were reared with the luxS mutant revealed few differences in the metabolite contents relative to diets with flies and wild-type bacteria (data not shown). As shown in FIG. 13, the most obvious effect was a dramatic increase in the SRH content of the diet. luxS is the second gene in the 2-step conversion of SAH to homocysteine via the SRH intermediate. The accumulation of SRH in the diet is consistent with the luxS-dependent conversion of SRH to homocysteine. A unifying but untested explanation for the lifespan influence of these two mutants is that the pdxBK mutant shortened fly lifespan by functioning as a source of methionine; whereas the SRH accumulation in diets that were inoculated with the luxS mutant functioned as a methionine sink. Together, these observations connect bacteria-dependent shifts in Drosophila melanogaster methionine cycle metabolites and lifespan, and suggest that control of bacterial gene activities to restrict dietary methionine might enable extension of Drosophila melanogaster lifespan.

### Bacterial Transsulfuration Extends Fruit Fly Lifespan

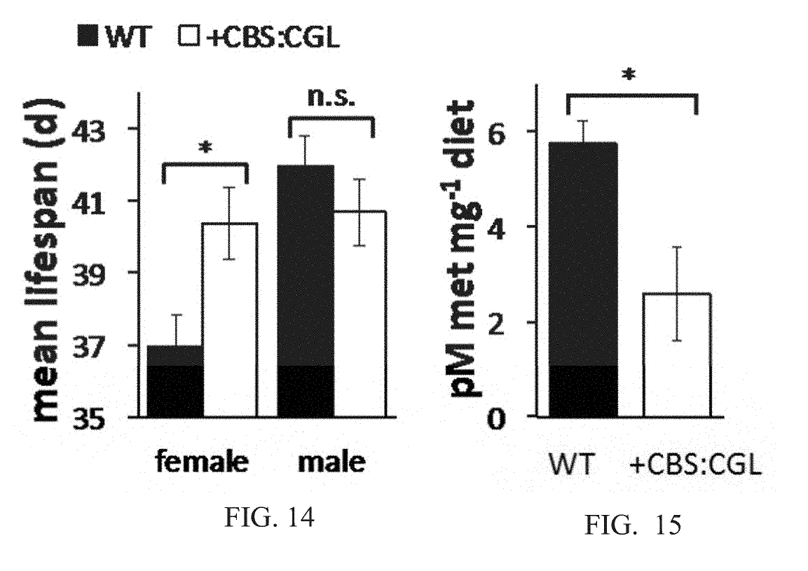

FIG. 14 shows mean lifespan differences of flies reared bearing CBS::CGL-expressing bacteria as compared to their wild-type counterparts, where CBS::CGL-expressing bacteria live 10% longer than their wild-type counterparts. The indication “*” represents that p<0.05. The following abbreviation are understood to mean: “CBS” is cystathionine-β-synthase and “CGL” is cystathionine-γ-lyase.

FIG. 15 shows effects on the methionine content of diets administered to bearing flies reared CBS::CGL-expressing bacteria as compared to their wild-type counterparts, where CBS::CGL expression reduces the methionine content of the diet by ˜66%. The indication “*” represents that p<0.05.

To test the idea that bacterial gene expression could be used to restrict dietary methionine in the flies, we monoassociated the flies with an A. fabarum strain that ectopically expressed transsulfuration genes CBS and CGL (+CBS:CGL). As shown in FIG. 15, the methionine content of diets on which flies bearing the +CBS:CGL mutant were lower than that of diets on which flies inoculated with wild-type A. fabarum were reared. Further as shown in FIG. 15, female, but not male, flies lived significantly longer when inoculated with +CBS:CGL. Together, these data demonstrate a correlation between the dietary-restricting activity of the +CBS:CGL mutant and lifespan extension that is consistent with the known lifespan-extending influence of methionine restriction. The data also further reinforce the link between bacterial methionine metabolism and host lifespan.

### Intermediate Discussion

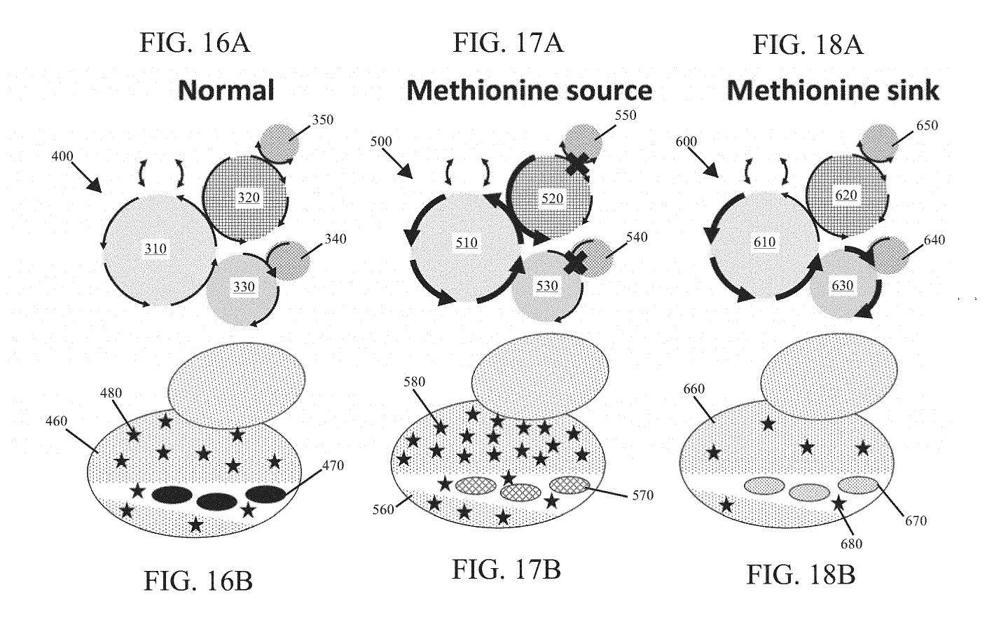

Under normal circumstances as shown in FIGS. 16A and 16b, microorganisms 470 in a gastrointestinal tract 460 with a methionine pathway 400 having normal points 310,320,330,340,350 both consume and produce methionine 480 such that there is a concentration of methionine 480.

As shown in FIGS. 17A and 17B, a pdxBK mutant 570 can have a methionine pathway 500 where transsulfuration at points 520 and 550 or 530 and 540 since there is a reduction in levels of vitamin B6, an essential cofactor in these processes. This can lead to increased methionine recycling 510, where the pdxBK mutant 570 produces more methionine 580 than it consumes. In this situation, the concentration of methionine 580 in a gastrointestinal tract 560 can be significantly greater than the gastrointestinal tract 460 under normal circumstances. This can lead to a shortened lifespan.

As shown in FIGS. 18A and 18, microorganisms 670 with enhance expression transsulfuration genes or that produce SRH but cannot convert it to homocysteine can drive flux from the methionine cycle 600 to other metabolites 620,630,640,650. These microorganisms 670 can sinking some of the methionine 680 and restricting its availability in the gastrointestinal tract 660, which can increase lifespans.

### The Probiotic Impact of a Recombinant Lactobacillus Strain

To create a strain of Lactobacillus that expresses CBS and CGL (ProL), we obtain and modify backbone integration vectors specific to Lactobacillus species. Since the current integration plasmids do not support constitutive expression, we modify the vectors to express the CBS and CGL genes behind the lactate dehydrogenase promoter (ldhL), a promoter that is constitutively active in Lactobacillus species (GEOFFROY, '00). We then clone the CBS and CGL genes into the newly constructed Lactobacillus integration vector and electroporate the vector into the Lactobacillus rhamnosus GG. We then perform Sanger sequencing to confirm the sequence and integration of the cloned genes. Together, these approaches create a recombinant strain of Lactobacillus rhamnosus GG that constitutively expresses the CBS and CGL genes stably from the chromosome. We also create a control strain to be used in all subsequent experiments by introducing the empty vector to Lactobacillus rhamnosus GG.

### Lifespan Effects of Strains in Flies

To test if ProL influences fruit fly lifespan we inoculate it to bacteria-free flies and measure their lifespan. To make mono-associated flies, Drosophila melanogaster eggs are surface-sterilized in bleach for 5 min., transferred to sterile diet in a sterile biosafety cabinet (˜30-50 eggs/vial), and are inoculated with the bacteria individually (NEWELL, '14, KOYLE, '16). Bacteria are cultured separately in mMRS liquid and solid media (NEWELL, '14), normalized to 0.1 OD₆₀₀ and 50 μl are added to the sterile diet containing sterile eggs. To measure fruit fly lifespan, flies are transferred 2-3 times weekly to fresh diet. Diet transfers are frequent because the flies are actively mating and developing offspring affect diet quality. At each transfer the number and sex of dead flies is recorded. All work is done with sterile diets in a biosafety cabinet to ensure no contaminating microbes are introduced during transfer. Differences in fly lifespan are tested using a Cox mixed effects survival model (THERNEAU, '12, THERNEAU, '14) in R.

### Metabolomic Effects of Strains in Flies

To determine whether ProL influences the Lactobacillus metabolome similarly to our recombinant Acetobacter strain, we measure the metabolomes of flies and their diets when inoculated with ProL versus control strains. At 7 and 30 days of age, 30 sex-separated flies are flash frozen on liquid nitrogen and undergo whole-metabolome analysis, together with samples from bacteria-free controls (48 samples total: 6 replicates for each of 2 ages, 2 sexes, 2 treatments). 30 mg of spent fly diet are flash-frozen and sent for analysis (24 samples total; 6 replicates, 2 ages, 2 treatments). ProL extends lifespan through methionine restriction since the methionine content of flies and diet is lower when inoculated with ProL versus the empty-vector control strain.

### Lifespan and Metabolomic Impacts of Strains Administered as a Probiotic

To determine the effect of a probiotic strain when inoculated to flies bearing an established microbial community, we inoculate flies with either the control or recombinant Acetobacter or Lactobacillus strains (4 treatments total: 2 test strains each with their own control), and measure their lifespan. At 5 and 30 days of age we collect diet and fly samples for metabolomic analysis as in our experiments above (144 samples total: 6 replicates for each of 2 ages, 2 sexes, 4 treatments+48 diet treatments). Further, we monitor persistence of ProL and the control throughout the experiment, and perform reinoculations on at least a weekly basis if either is lost from the flies over time. Any strain that acts as a probiotic extends the lifespan of bacteria-associated flies. Since there can be differences in how Acetobacter and Lactobacillus persist in the flies, we include the recombinant Acetobacter strain to control for differences in persistence on the probiotic effect.

Together, the approaches as described here create a candidate probiotic strain that has lifespan-extending effects in mammals. ProL functions similarly to the Acetobacter probiotic strain (FIGS. 14 and 15) in decreasing the methionine content of the diet and the flies and extending fly lifespan.

### Gerobiotic Effects in Mammals.

Bacteria can generally extend fly lifespan by methionine restriction and dietary methionine restrictions in mice extends mouse lifespans and alters the abundances of several serum biomarkers and other indicators of mouse health in 5-week old mice. Thus, bacteria extend mouse lifespans through methionine restriction which can be detected via methionine-restriction-like biomarkers in 8-week old mice fed the bacteria.

To presumptively assess if ProL administration mimics methionine restriction in mice, we feed ProL or the control strain (10⁹ cfu) to each of 20 mice (5 mice per cage) in drinking water daily for 8 weeks while measuring methionine-restriction indicators on a weekly basis or at the end of the experiment as shown in Table 5 below. Table 5 shows Phenotypes to measure in probiotic-administered mice and controls. Over the indicated time periods these are methionine-restriction responsive phenotypes, as reported in (LEES, '14).

TABLE 5

| Phenotype | Measured |
|---|---|
| Body Weight | Weekly |
| Food Intake | Weekly |
| Fecal Microbiome | Weekly |
| Glucose Tolerance | 8 Weeks |
| Blood Glucose | 8 Weeks |
| Serum Insulin | 8 Weeks |
| Serum Triglyceride | 8 Weeks |
| Liver Triglyceride | 8 Weeks |
| Liver Gene Expression | 8 Weeks |
| White Adipose Tissue Gene Expression | 8 Weeks |

We use 3-week-old male C57BL/6J wild-type mice for these experiments. At the end of the experiment, blood samples are collected from each mouse, the mice are sacrificed, and liver and white adipose tissue are dissected from each animal within 1 hour of sacrifice. Serum, liver, and adipose biomarker levels are assessed in triplicate from each mouse sample as described previously (LEES, '14). Additionally, to determine gene expression in tissues that display distinctive methionine-restriction-like signatures, dissected tissues from the mice in each cage are pooled and gene expression in the sample analyzed by RNAseq at a depth of 40 million reads/sample (see also our published and current RNAseq work in Drosophila, e.g. (DOBSON, '16)), for a total of 5 replicates per treatment. Tissues analyzed are the liver and white adipose tissue. We collect fecal samples weekly to monitor the impact of the probiotic administration on the mouse microbiome on an individual mouse basis by a 16S rRNA marker gene survey as in our ongoing work (performed as described in (KOZICH, '13)).

ProL leads to methionine restriction in the mice since they eat more but weigh less, have greater glucose tolerance/insulin responsiveness, and have lower blood glucose, serum insulin, serum triglyceride, and liver triglyceride levels. Also, the bacteria persist and are abundant as indicated in 16S data. Taken together, these experiments reveal the probiotic potential of ProL.

### REFERENCES

Blum, J E, C N Fischer, J Miles, J Handelsman. 2013. Frequent replenishment sustains the beneficial microbiome of drosophila melanogaster. MBio 4:e00860-13.
Broderick, N A, N Buchon, B Lemaitre. 2014. Microbiota-induced changes in drosophila melanogaster host gene expression and gut morphology. MBio 5:e01117-14.
Brummel, T, A Ching, L Seroude, A F Simon, S Benzer. 2004. Drosophila lifespan enhancement by exogenous bacteria. Proc Natl Acad Sci USA 101:12974-9.
Bushey, D, K A Hughes, G Tononi, C Cirelli. 2010. Sleep, aging, and lifespan in drosophila. BMC Neurosci 11:56.
Chandler, J A, J M Lang, S Bhatnagar, J A Eisen, A Kopp. 2011. Bacterial communities of diverse drosophila species: Ecological context of a host-microbe model system. Plos Genetics 7:e1002272.
Chaston, J M, P D Newell, A E Douglas. 2014. Metagenome-wide association of microbial determinants of host phenotype in drosophila melanogaster. MBio 5:e01631-14.
Core Team, R. 2016. R: A language and environment for statistical computing, R Foundation for Statistical Computing, Vienna, Austria. http://www.R-project.org.
Cox, C R, M S Gilmore. 2007. Native microbial colonization of drosophila melanogaster and its use as a model of enterococcus faecalis pathogenesis. Infection and Immunity 75:1565-1576.
Deshpande, S A, R Yamada, C M Mak, B Hunter, A Soto Obando, S Hoxha, W W Ja. 2015. Acidic food ph increases palatability and consumption and extends drosophila lifespan. J Nutr 145:2789-96.
Dobson, A J, J M Chaston, A E Douglas. 2016. The drosophila transcriptional network is structured by microbiota. B 17:975.
Geoffroy, M C, C Guyard, B Quatannens, S Pavan, M Lange, A Mercenier. 2000. Use of green fluorescent protein to tag lactic acid bacterium strains under development as live vaccine vectors. Appl Environ Microbiol 66:383-91.
Grandison, R C, M D Piper, L Partridge. 2009. Amino-acid imbalance explains extension of lifespan by dietary restriction in drosophila. Nature 462:1061-4.
Helfand, S L, B Rogina. 2003. From genes to aging in drosophila. Adv Genet 49:67-109.
Kabil, H, O Kabil, R Banerjee, L G Harshman, S D Pletcher. 2011. Increased transsulfuration mediates longevity and dietary restriction in drosophila. Proc Natl Acad Sci USA 108:16831-6.
Koyle, M L, M Veloz, A M Judd, A C Wong, P D Newell, A E Douglas, J M Chaston. 2016. Rearing the fruit fly drosophila melanogaster under axenic and gnotobiotic conditions. J Vis Exp doi:10.3791/54219.
Kozich, J J, S L Westcott, N T Baxter, S K Highlander, P D Schloss. 2013. Development of a dual-index sequencing strategy and curation pipeline for analyzing amplicon sequence data on the miseq illumina sequencing platform. Appl Environ Microbiol 79:5112-20.
Koziel, R, C Ruckenstuhl, E Albertini, M Neuhaus, C Netzberger, M Bust, F Madeo, R J Wiesner, P Jansen-Durr. 2014. Methionine restriction slows down senescence in human diploid fibroblasts. Aging Cell 13:1038-48.
Lee, B C, A Kaya, S Ma, G Kim, M V Gerashchenko, S H Yim, Z Hu, L G Harshman, V N Gladyshev. 2014. Methionine restriction extends lifespan of drosophila melanogaster under conditions of low amino-acid status. Nat Commun 5:3592.
Lees, E K, E Krol, L Grant, K Shearer, C Wyse, E Moncur, A S Bykowska, N Mody, T W Gettys, M Delibegovic. 2014. Methionine restriction restores a younger metabolic phenotype in adult mice with alterations in fibroblast growth factor 21. Aging Cell 13 :817-27.
Mclsaac, R S, K N Lewis, P A Gibney, R Buffenstein. 2016. From yeast to human: Exploring the comparative biology of methionine restriction in extending eukaryotic life span. Ann NY Acad Sci 1363:155-70.
Newell, P D, A E Douglas. 2014. Interspecies interactions determine the impact of the gut microbiota on nutrient allocation in drosophila melanogaster. Appl Environ Microbiol 80:788-96.
Obata, F, M Miura. 2015. Enhancing s-adenosyl-methionine catabolism extends drosophila lifespan. Nat Commun 6:8332.
Orentreich, N, J R Matias, A DeFelice, J A Zimmerman. 1993. Low methionine ingestion by rats extends life span. J Nut 123 :269-74.
Ottaviani, E, N Ventura, M Mandrioli, M Candela, A Franchini, C Franceschi. 2011. Gut microbiota as a candidate for lifespan extension: An ecological/evolutionary perspective targeted on living organisms as metaorganisms. Biogerontology 12:599-609.
Parkhitko, A A, R Binari, N Zhang, J M Asara, F Demontis, N Perrimon. 2016. Tissue-specific down-regulation of s-adenosyl-homocysteine via suppression of dahcyl1/dahcyl2 extends health span and life span in drosophila. Genes Dev doi:10.1101/gad.282277.116.
Rogers, G B, J Kozlowska, J Keeble, K Metcalfe, M Fao, S E Dowd, A J Mason, M A McGuckin, K D Bruce. 2014. Functional divergence in gastrointestinal microbiota in physically-separated genetically identical mice. Science Reports 4:5437.
Sexton, C E, H Z Smith, P D Newell, A E Douglas, J M Chaston. 2018. Magnamwar: An r package for genome-wide association studies of bacterial orthologs. Bioinformatics doi:10.1093/bioinformatics/bty001.
Shaw, P, K Ocorr, R Bodmer, S Oldham. 2008. Drosophila aging 2006/2007. Exp Gerontol 43:5-10.
Therneau, T. 2012. Mixed effects cox models, v2.2-3.
Therneau, T. 2014. A package for survival analysis in s, v2.37-7. http://cran.r-project.org/package=survival.
Troen, A M, E E French, J F Roberts, J Selhub, J M Ordovas, L D Parnell, C Q Lai. 2007. Lifespan modification by glucose and methionine in drosophila melanogaster fed a chemically defined diet. Age (Dordr) 29:29-39.
Unckless, R L, S M Rottschaefer, B P Lazzaro. 2015. A genome-wide association study for nutritional indices in drosophila. G3 (Bethesda) doi:10.1534/g3.114.016477.
Uthus, E O, H M Brown-Borg. 2006. Methionine flux to transsulfuration is enhanced in the long living ames dwarf mouse. Mech Ageing Dev 127:444-50.
White, K M, M K Matthews, R C Hughes, A J Sommer, J S Griffitts, P D Newell, J M Chaston. 2018. A metagenome-wide association study and arrayed mutant library confirm acetobacter lipopolysaccharide genes are necessary for association with drosophila melanogaster. G3 (Bethesda) 8:1119-1127.
Wong, A C, J M Chaston, A E Douglas. 2013. The inconstant gut microbiota of drosophila species revealed by 16s rrna gene analysis. ISMS J 7:1922-32.
Wong, A C, A J Dobson, A E Douglas. 2014. Gut microbiota dictates the metabolic response of drosophila to diet. J Exp Biol 217:1894-901.
Wong, C N, P Ng, A E Douglas. 2011. Low-diversity bacterial community in the gut of the fruitfly drosophila melanogaster. Environ Microbiol 13:1889-900.
Yamada, R, S A Deshpande, K D Bruce, E M Mak, W W Ja. 2015. Microbes promote amino acid harvest to rescue undernutrition in drosophila. Cell Rep doi:10.1016/j.celrep.2015.01.018.
Yamada, R, S A Deshpande, E S Keebaugh, M R Ehrlich, A Soto Obando, W W Ja. 2017. Mifepristone reduces food palatability and affects drosophila feeding and lifespan. J Gerontol A Biol Sci Med Sci 72:173-180.
Zhang, C, S Li, L Yang, P Huang, W Li, S Wang, G Zhao, M Zhang, X Pang, Z Yan, Y Liu, L Zhao. 2013. Structural modulation of gut microbiota in life-long calorie-restricted mice. Nat Commun 4:2163.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A probiotic bacterium comprising a heterologous polynucleotide encoding a cystathionine-β-synthase or a cystathionine-γ-lyase and operably linked to a promoter polynucleotide, wherein the probiotic bacterium is derived from a bacteria strain incapable of reverse transsulfurylation.

2. The probiotic bacterium of claim 1, wherein the cystathionine-β-synthase has an amino acid sequence that is at least about 30% identical to any one or more of SEQ ID NO: 1, SEQ ID NO: 20, SEQ ID NO: 22, SEQ ID NO: 26, SEQ ID NO: 30, SEQ ID NO: 34, SEQ ID NO: 36, SEQ ID NO: 50, SEQ ID NO: 58, or SEQ ID NO: 74.

3. The probiotic bacterium of claim 1, wherein the cystathionine-γ-lyase has an amino acid sequence that is at least about 30% identical to any one or more of SEQ ID NO: 3, SEQ ID NO: 24, SEQ ID NO: 28, SEQ ID NO: 32, SEQ ID NO: 38, SEQ ID NO: 40, SEQ ID NO: 52, or SEQ ID NO: 76.

4. The probiotic bacterium of claim 1, wherein the promoter polynucleotide is a constitutive promoter polynucleotide.

5. The probiotic bacterium of claim 1 further comprising a mutation in a homologous polynucleotide that encodes a protein selected from at least one of a S-ribosylhomocysteine lyase, methionine synthase, homoserine O-acetyltransferase, 5-methyltetrahydropteroyltriglutamate-homocysteine methyltransferase, and a cobalamin synthase W domain-containing protein 1 or in a homologous promoter polynucleotide operably linked to the homologous polynucleotide, wherein the mutation reduces activity or expression of the protein.

6. The probiotic bacterium of claim 1, wherein the probiotic bacterium belongs to a genus Lactobacillus, Bifidobacterium, Escherichia, Enterococcus, Bacillus, Propionibacterium, Streptococcus, Lactococcus, Pediococcus, or Saccharomyces or belongs to an order Lactobacillales.

7. A composition for reducing methionine content of a diet of a subject or extending a lifespan of the subject comprising probiotic bacteria of claim 1 and a pharmaceutically acceptable excipient.

8. A probiotic bacterium comprising a constitutive promoter polynucleotide operably linked to a polynucleotide encoding a cystathionine-γ-synthase, a cystathionine-β-lyase, or a adenosylhomocysteine nucleosidase.

9. The probiotic bacterium of claim 8, wherein the polynucleotide encoding cystathionine-γ-synthase, cystathionine-β-lyase, or adenosylhomocysteine nucleosidase is a heterologous polynucleotide.

10. The probiotic bacterium of claim 9, wherein the cystathionine-γ-synthase has an amino acid sequence that is at least about 30% identical to any one or more of SEQ ID NO: 5, SEQ ID NO: 44, SEQ ID NO: 56, SEQ ID NO: 68, SEQ ID NO: 70, SEQ ID NO: 72, SEQ ID NO: 82, SEQ ID NO: 84, SEQ ID NO: 86, SEQ ID NO: 90, SEQ ID NO: 92, or SEQ ID NO: 106.

11. The probiotic bacterium of claim 9, wherein the cystathionine-β-lyase has an amino acid sequence that is at least about 30% identical to any one or more of SEQ ID NO: 7, SEQ ID NO: 42, SEQ ID NO: 48, SEQ ID NO: 54, SEQ ID NO: 60, SEQ ID NO: 62, SEQ ID NO: 64, SEQ ID NO: 66, SEQ ID NO: 78, SEQ ID NO: 80, SEQ ID NO: 88, SEQ ID NO: 94, SEQ ID NO: 98, SEQ ID NO: 100, SEQ ID NO: 102, SEQ ID NO: 104, SEQ ID NO: 110, SEQ ID NO: 112, or SEQ ID NO: 114.

12. The probiotic bacterium of claim 9, wherein the adenosylhomocysteine nucleosidase has an amino acid sequence that is at least about 30% identical to any one or more of SEQ ID NO: 9, SEQ ID NO: 46, SEQ ID NO: 96, SEQ ID NO: 108, or SEQ ID NO: 116.

13. The probiotic bacterium of claim 8 further comprising a heterologous polynucleotide encoding a cystathionine-β-synthase or a cystathionine-γ-lyase operably linked to a second promoter polynucleotide.

14. The probiotic bacterium of claim 8 further comprising a heterologous polynucleotide encoding a cystathionine-β-synthase or a cystathionine-γ-lyase operably linked to the constitutive promoter polynucleotide.

15. The probiotic bacterium of claim 8 further comprising a mutation in a homologous polynucleotide that encodes a protein selected from at least one of a S-ribosylhomocysteine lyase, methionine synthase, homoserine O-acetyltransferase, 5-methyltetrahydropteroyltriglutamate-homocysteine methyltransferase, and a cobalamin synthase W domain-containing protein 1 or in a homologous promoter polynucleotide operably linked to the homologous polynucleotide, wherein the mutation reduces activity or expression of the protein.

16. The probiotic bacterium of claim 8, wherein the probiotic bacterium belongs to a genus Lactobacillus, Bifidobacterium, Escherichia, Enterococcus, Bacillus, Propionibacterium, Streptococcus, Lactococcus, Pediococcus, or Saccharomyces or belongs to an order Lactobacillales.

17. A composition for reducing methionine content of a diet of a subject or extending a lifespan of the subject comprising probiotic bacteria of claim 8 and a pharmaceutically acceptable excipient.

18. A probiotic bacterium comprising a mutation in a homologous polynucleotide that encodes a protein selected from at least one of a S-ribosylhomocysteine lyase, methionine synthase, homoserine O-acetyltransferase, 5-methyltetrahydropteroyltriglutamate-homocysteine methyltransferase, and a cobalamin synthase W domain-containing protein 1 or in a homologous promoter polynucleotide operably linked to the homologous polynucleotide, wherein the mutation reduces activity or expression of the protein.

19. The probiotic bacterium of claim 18, wherein the probiotic bacterium belongs to a genus Lactobacillus, Bifidobacterium, Escherichia, Enterococcus, Bacillus, Propionibacterium, Streptococcus, Lactococcus, Pediococcus, or Saccharomyces or belongs to an order Lactobacillales.

20. A composition for reducing methionine content of a diet of a subject or extending a lifespan of the subject comprising probiotic bacteria of claim 18 and a pharmaceutically acceptable excipient.
